SEQUENCE LISTING (204 sequences)

SEQ ID NO: 1 - DNA, 636 bp, Artificial Sequence
  Description of Artificial Sequence Synthetic polynucleotide
  misc_feature (28)..(162): GN168 lysin
  CDS (28)..(612)
gtttaactttaagaaggagaattcaccatgaggttaaaaatggcacgaagaagatacagacttccgcgacgtagaagtcgaagacttttttcaagaactgcattgaggatgcatccaagaaataggcttcgaagaattatgcgtggcggcattaggttcaccgcgggcggcaccgcgggcggccgtacatcccaacgaggcatcgacctcatcaaatccttcgagggcctgcgcctgtccgcttaccaggactcggtgggtgtctggaccataggttacggcaccactcggggcgtcacccgctacatgacgatcaccgtcgagcaggccgagcggatgctgtcgaacgacattcagcgcttcgagccagagctagacaggctggcgaaggtgccactgaaccagaaccagtgggatgccctgatgagcttcgtgtacaacctgggcgcggccaatctggcgtcgtccacgctgctcgacctgctgaacaagggtgactaccagggagcagcggaccagttcccgcattgggtgaatgcgggcggtaagcgcttggatggtctggttaagcgtcgagcagccgagcgtgcgctgttcctggagccactatcgtgataaaagcttggctgttttggc

SEQ ID NO: 2 - protein, 195 aa, Artificial Sequence
  Synthetic Construct
MRLKMARRRYRLPRRRSRRLFSRTALRMHPRNRLRRIMRGGIRFTAGGTAGGRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLDLLNKGDYQGAADQFPHWVNAGGKRLDGLVKRRAAERALFLEPLS

SEQ ID NO: 3 - DNA, 567 bp, Artificial Sequence
  Description of Artificial Sequence Synthetic polynucleotide
  CDS (28)..(543)
  misc_feature (29)..(543): GN 176 lysin
gtttaactttaagaaggagaattcaccatgagctttaacgtgaccccgaaatttaaacgctggcagctgtattttcgcggccgcatgtggaccgcgggcggcaccgcgggcggccgtacatcccaacgaggcatcgacctcatcaaatccttcgagggcctgcgcctgtccgcttaccaggactcggtgggtgtctggaccataggttacggcaccactcggggcgtcacccgctacatgacgatcaccgtcgagcaggccgagcggatgctgtcgaacgacattcagcgcttcgagccagagctagacaggctggcgaaggtgccactgaaccagaaccagtgggatgccctgatgagcttcgtgtacaacctgggcgcggccaatctggcgtcgtccacgctgctcgacctgctgaacaagggtgactaccagggagcagcggaccagttcccgcattgggtgaatgcgggcggtaagcgcttggatggtctggttaagcgtcgagcagccgagcgtgcgctgttcctggagccactatcgtgataaaagcttggctgttttggc

SEQ ID NO: 4 - protein, 172 aa, Artificial Sequence
  Synthetic Construct
MSFNVTPKFKRWQLYFRGRMWTAGGTAGGRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLDLLNKGDYQGAADQFPHWVNAGGKRLDGLVKRRAAERALFLEPLS

SEQ ID NO: 5 - DNA, 582 bp, Artificial Sequence
  Description of Artificial Sequence Synthetic polynucleotide
  misc_feature (28)..(558): GN178 lysin
  CDS (28)..(558)
gtttaactttaagaaggagaattcaccatgccaccaatttttagcaaactggcgggcaaaaaaattaaaaacctgctgattagcggcctgaaaggcggtagcggcagcggtagcggtagcggcagcccgcgtacatcccaacgaggcatcgacctcatcaaatccttcgagggcctgcgcctgtccgcttaccaggactcggtgggtgtctggaccataggttacggcaccactcggggcgtcacccgctacatgacgatcaccgtcgagcaggccgagcggatgctgtcgaacgacattcagcgcttcgagccagagctagacaggctggcgaaggtgccactgaaccagaaccagtgggatgccctgatgagcttcgtgtacaacctgggcgcggccaatctggcgtcgtccacgctgctcgacctgctgaacaagggtgactaccagggagcagcggaccagttcccgcattgggtgaatgcgggcggtaagcgcttggatggtctggttaagcgtcgagcagccgagcgtgcgctgttcctggagccactatcgtgataaaagcttggctgttttggc

SEQ ID NO: 6 - protein, 177 aa, Artificial Sequence
  Synthetic Construct
MPPIFSKLAGKKIKNLLISGLKGGSGSGSGSGSPRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLDLLNKGDYQGAADQFPHWVNAGGKRLDGLVKRRAAERALFLEPLS

SEQ ID NO: 7 - DNA, 429 bp, Artificial Sequence
  Description of Artificial Sequence Synthetic polynucleotide
  misc_feature (28)..(405): GN217 lysin
  CDS (28)..(405)
gtttaactttaagaaggagaattcaccatgacctacaccctgtctaaacgttctctggacaacctgaaaggtgttcacccggacctggttgctgttgttcaccgtgctatccagctgaccccggttgacttcgctgttatcgaaggtctgcgttctgtttctcgtcagaaagaactggttgctgctggtgcttctaaaaccatgaactctcgtcacctgaccggtcacgctgttgacctggctgcttacgttaacggtatccattgggactggccgctgtacgacgctatcgctgttgctgttaaagctgctgctaaagaactgggtgttgctatcgtttggggtggtgactggaccaccttcaaagacggtccgcacttcgaactggaccgttctaaataccgttaataaaagcttggctgttttggc

SEQ ID NO: 8 - protein, 126 aa, Artificial Sequence
  Synthetic Construct
MTYTLSKRSLDNLKGVHPDLVAVVHRAIQLTPVDFAVIEGLRSVSRQKELVAAGASKTMNSRHLTGHAVDLAAYVNGIHWDWPLYDAIAVAVKAAAKELGVAIVWGGDWTTFKDGPHFELDRSKYR

SEQ ID NO: 9 - DNA, 501 bp, Artificial Sequence
  Description of Artificial Sequence Synthetic polynucleotide
  misc_feature: GN218 lysin
  CDS (28)..(477)
gtttaactttaagaaggagaattcaccatgacctacaccctgtctaaacgttctctggacaacctgaaaggtgttcacccggacctggttgctgttgttcaccgtgctatccagctgaccccggttgacttcgctgttatcgaaggtctgcgttctgtttctcgtcagaaagaactggttgctgctggtgcttctaaaaccatgaactctcgtcacctgaccggtcacgctgttgacctggctgcttacgttaacggtatccgttgggactggccgctgtacgacgctatcgctgttgctgttaaagctgctgctaaagaactgggtgttgctatcgtttggggtggtgactggaccaccttcaaagacggtccgcacttcgaactggaccgttctaaatacggcggtggctctggaggtggtgggtccggcggtggctctcgcctgaaaaaaattggcaaagtgctgaaatggatttaataaaagcttggctgttttggc

SEQ ID NO: 10 - protein, 150 aa, Artificial Sequence
  Synthetic Construct
MTYTLSKRSLDNLKGVHPDLVAVVHRAIQLTPVDFAVIEGLRSVSRQKELVAAGASKTMNSRHLTGHAVDLAAYVNGIRWDWPLYDAIAVAVKAAAKELGVAIVWGGDWTTFKDGPHFELDRSKYGGGSGGGGSGGGSRLKKIGKVLKWI

SEQ ID NO: 11 - DNA, 573 bp, Artificial Sequence
  Description of Artificial Sequence Synthetic polynucleotide
  misc_feature (28)..(549): GN223 lysin
  CDS (28)..(549)
gtttaactttaagaaggagaattcaccatgacctacaccctgtctaaacgttctctggacaacctgaaaggtgttcacccggacctggttgctgttgttcaccgtgctatccagctgaccccggttgacttcgctgttatcgaaggtctgcgttctgtttctcgtcagaaagaactggttgctgctggtgcttctaaaaccatgaactctcgtcacctgaccggtcacgctgttgacctggctgcttacgttaacggtatccgttgggactggccgctgtacgacgctatcgctgttgctgttaaagctgctgctaaagaactgggtgttgctatcgtttggggtggtgactggaccaccttcaaagacggtccgcacttcgaactggaccgttctaaataccgtccaccaggcggtggctctggaggtggtgggtccggcggtggctcttcgaagaaggcgtcgaggaagagttttactaagggtgccgttaaggttcataagaaaaatgttcctactcgtgttcctatgcgtggcggtattaggctttaataaaagcttggctgttttggc

SEQ ID NO: 12 - protein, 174 aa, Artificial Sequence
  Synthetic Construct
MTYTLSKRSLDNLKGVHPDLVAVVHRAIQLTPVDFAVIEGLRSVSRQKELVAAGASKTMNSRHLTGHAVDLAAYVNGIRWDWPLYDAIAVAVKAAAKELGVAIVWGGDWTTFKDGPHFELDRSKYRPPGGGSGGGGSGGGSSKKASRKSFTKGAVKVHKKNVPTRVPMRGGIRL

SEQ ID NO: 13 - DNA, 519 bp, Artificial Sequence
  Description of Artificial Sequence Synthetic polynucleotide
  misc_feature (28)..(495): GN239 lysin
  CDS (28)..(495)
gtttaactttaagaaggagaattcaccatgacctacaccctgtctaaacgttctctggacaacctgaaaggtgttcacccggacctggttgctgttgttcaccgtgctatccagctgaccccggttgacttcgctgttatcgaaggtctgcgttctgtttctcgtcagaaagaactggttgctgctggtgcttctaaaaccatgaactctcgtcacctgaccggtcacgctgttgacctggctgcttacgttaacggtatccgttgggactggccgctgtacgacgctatcgctgttgctgttaaagctgctgctaaagaactgggtgttgctatcgtttggggtggtgactggaccaccttcaaagacggtccgcacttcgaactggaccgttctaaatacggcggtggctctggaggtggtgggtccggcggtggctctcgtaaaaaaacccgtaaacgtctgaaaaaaatcggtaaagttctgaaatggatctaataaaagcttggctgttttggc

SEQ ID NO: 14 - protein, 156 aa, Artificial Sequence
  Synthetic Construct
MTYTLSKRSLDNLKGVHPDLVAVVHRAIQLTPVDFAVIEGLRSVSRQKELVAAGASKTMNSRHLTGHAVDLAAYVNGIRWDWPLYDAIAVAVKAAAKELGVAIVWGGDWTTFKDGPHFELDRSKYGGGSGGGGSGGGSRKKTRKRLKKIGKVLKWI

SEQ ID NO: 15 - DNA, 570 bp, Artificial Sequence
  Description of Artificial Sequence Synthetic polynucleotide
  misc_feature (28)..(546): GN243 lysin
  CDS (28)..(546)
gtttaactttaagaaggagaattcaccatgacctacaccctgtctaaacgttctctggacaacctgaaaggtgttcacccggacctggttgctgttgttcaccgtgctatccagctgaccccggttgacttcgctgttatcgaaggtctgcgttctgtttctcgtcagaaagaactggttgctgctggtgcttctaaaaccatgaactctcgtcacctgaccggtcacgctgttgacctggctgcttacgttaacggtatccgttgggactggccgctgtacgacgctatcgctgttgctgttaaagctgctgctaaagaactgggtgttgctatcgtttggggtggtgactggaccaccttcaaagacggtccgcacttcgaactggaccgttctaaataccgtaaaaaaacccgtaaacgtctgaaaaaaatcggtaaagttctgaaatggatcccaccaggcggtggctctggaggtggtgggtccggcggtggctctacccgcaaacgcctgaaaaaaattggcaaagtgctgaaatggatttaataaaagcttggctgttttggc

SEQ ID NO: 16 - protein, 173 aa, Artificial Sequence
  Synthetic Construct
MTYTLSKRSLDNLKGVHPDLVAVVHRAIQLTPVDFAVIEGLRSVSRQKELVAAGASKTMNSRHLTGHAVDLAAYVNGIRWDWPLYDAIAVAVKAAAKELGVAIVWGGDWTTFKDGPHFELDRSKYRKKTRKRLKKIGKVLKWIPPGGGSGGGGSGGGSTRKRLKKIGKVLKWI

SEQ ID NO: 17 - DNA, 528 bp, Artificial Sequence
  Description of Artificial Sequence Synthetic polynucleotide
  misc_feature (28)..(504): GN280 lysin
  CDS (28)..(504)
gtttaactttaagaaggagaattcaccatgaaactcagcgaaaaacgagcactgttcacccagctgcttgcccagttaattctttgggcaggaactcaggatcgagtgtcagtagccttggatcaagtgaaaaggacacaggctgaagctgatgccaatgctaagtctggagcaggcattaggaactctctccatctactgggattagccggtgatcttatcctctacaaggatggtaaatacatggataagagcgaggattataagttcctgggagattactggaagagtctccatcctctttgtcggtggggcggagattttaaaagccgtcctgatggtaatcatttctccttggaacacgaaggagtgcaacgtaaaaaaacccgtaaacgtctgaaaaaaatcggtaaagttctgaaatggatcccaccaaccgcgggcggcaccgcgggcggcacccgcaaacgcctgaaaaaaattggcaaagtgctgaaatggatttaataaaagcttggctgttttggc

SEQ ID NO: 18 - protein, 159 aa, Artificial Sequence
  Synthetic Construct
MKLSEKRALFTQLLAQLILWAGTQDRVSVALDQVKRTQAEADANAKSGAGIRNSLHLLGLAGDLILYKDGKYMDKSEDYKFLGDYWKSLHPLCRWGGDFKSRPDGNHFSLEHEGVQRKKTRKRLKKIGKVLKWIPPTAGGTAGGTRKRLKKIGKVLKWI

SEQ ID NO: 19 - DNA, 543 bp, Artificial Sequence
  Description of Artificial Sequence Synthetic polynucleotide
  misc_feature: GN281 lysin
  CDS (28)..(519)
gtttaactttaagaaggagaattcaccatgaaactcagcgaaaaacgagcactgttcacccagctgcttgcccagttaattctttgggcaggaactcaggatcgagtgtcagtagccttggatcaagtgaaaaggacacaggctgaagctgatgccaatgctaagtctggagcaggcattaggaactctctccatctactgggattagccggtgatcttatcctctacaaggatggtaaatacatggataagagcgaggattataagttcctgggagattactggaagagtctccatcctctttgtcggtggggcggagattttaaaagccgtcctgatggtaatcatttctccttggaacacgaaggagtgcaacgtaaaaaaacccgtaaacgtctgaaaaaaatcggtaaagttctgaaatggatcggcggtggctctggaggtggtgggtccggcggtggctctccaccaacccgcaaacgcctgaaaaaaattggcaaagtgctgaaatggatttaataaaagcttggctgttttggc

SEQ ID NO: 20 - protein, 164 aa, Artificial Sequence
  Synthetic Construct
MKLSEKRALFTQLLAQLILWAGTQDRVSVALDQVKRTQAEADANAKSGAGIRNSLHLLGLAGDLILYKDGKYMDKSEDYKFLGDYWKSLHPLCRWGGDFKSRPDGNHFSLEHEGVQRKKTRKRLKKIGKVLKWIGGGSGGGGSGGGSPPTRKRLKKIGKVLKWI

SEQ ID NO: 21 - DNA, 852 bp, Artificial Sequence
  Description of Artificial Sequence Synthetic polynucleotide
  misc_feature: GN316 lysin
gaattcaccatgggatcccatcatcaccaccatcatggtgccattttaaagattggcagcaaaggtctggaagttaagaatcttcagaccagtctcaacaaaatcgggttcaatctggttgccgatggcatatttggtaaagcgactgacaacgccgtcagggcagttcaggcaggtgccggactggtcgttgatggtattgctggccccaagaccatgtatgcgattcgcaacgcaggggagtctcatcaggatcatctgactgaggctgacttgattgacgctgctcgtgaattgtctgttgaccttgctagcatcaaggcagtcaaccaagtagaatcgcgcggtactggcttcaccaagtctggtaagatcaagacattgtttgaacgccacatcatgtacaaaaagctgaatgccaagttcggtcaggcaaaagccaatgctctggcccagctttacccgacgttggttaacgccaaagccgggggatacacaggtggggacgcggagttggaacgactccatggtgcaatagcgatcgataaagattgcgcctacgagagcgcttcctacgggttattccagatcatggggttcaactgcgttatttgtggatatgacaatgccgaggagatgttcaacgactttctcactggtgaacgtgctcagctcatggcatttgtcaagttcatcaaggctgacgccaatctgtggaaagcattgaaggacaagaattgggctgagtttgctcggcgttacaatggcccggcgtatgcacagaaccagtacgacaccaagctggctgcagcatacaaatcattcagttagtaaaagcttggctgttttggc

SEQ ID NO: 22 - protein, 264 aa, Artificial Sequence
  Description of Artificial Sequence Synthetic polypeptide
  MISC_FEATURE (1)..(264): GN316 lysin
MAILKIGSKGLEVKNLQTSLNKIGFNLVADGIFGKATDNAVRAVQAGAGLVVDGIAGPKTMYAIRNAGESHQDHLTEADLIDAARELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVKFIKADANLWKALKDKNWAEFARRYNGPAYAQNQYDTKLAAAYKSFS

SEQ ID NO: 23 - DNA, 879 bp, Artificial Sequence
  Description of Artificial Sequence Synthetic polynucleotide
  misc_feature (1)..(879): modified GN316 lysin
gaattcaccatgggatcccatcatcaccaccatcatggtgccggatcccatcatcaccaccatcatggtattttaaagattggcagcaaaggtctggaagttaagaatcttcagaccagtctcaacaaaatcgggttcaatctggttgccgatggcatatttggtaaagcgactgacaacgccgtcagggcagttcaggcaggtgccggactggtcgttgatggtattgctggccccaagaccatgtatgcgattcgcaacgcaggggagtctcatcaggatcatctgactgaggctgacttgattgacgctgctcgtgaattgtctgttgaccttgctagcatcaaggcagtcaaccaagtagaatcgcgcggtactggcttcaccaagtctggtaagatcaagacattgtttgaacgccacatcatgtacaaaaagctgaatgccaagttcggtcaggcaaaagccaatgctctggcccagctttacccgacgttggttaacgccaaagccgggggatacacaggtggggacgcggagttggaacgactccatggtgcaatagcgatcgataaagattgcgcctacgagagcgcttcctacgggttattccagatcatggggttcaactgcgttatttgtggatatgacaatgccgaggagatgttcaacgactttctcactggtgaacgtgctcagctcatggcatttgtcaagttcatcaaggctgacgccaatctgtggaaagcattgaaggacaagaattgggctgagtttgctcggcgttacaatggcccggcgtatgcacagaaccagtacgacaccaagctggctgcagcatacaaatcattcagttagtaaaagcttggctgttttggc

SEQ ID NO: 24 - protein, 273 aa, Artificial Sequence
  Description of Artificial Sequence Synthetic polypeptide
  MISC_FEATURE (1)..(273): modified GN316 lysin
MGSHHHHHHGAILKIGSKGLEVKNLQTSLNKIGFNLVADGIFGKATDNAVRAVQAGAGLVVDGIAGPKTMYAIRNAGESHQDHLTEADLIDAARELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVKFIKADANLWKALKDKNWAEFARRYNGPAYAQNQYDTKLAAAYKSFS

SEQ ID NO: 25 - DNA, 612 bp, Pseudomonas phage KPP10
  misc_feature (28)..(588): GN329
  CDS (28)..(588)
gtttaactttaagaaggagaattcaccatgatcaccgacagagagtatcagcaagctgctgagatgttgggagtagatgtcccagcgatcaaggcagtgaccaaggtggaggccccggtagggggcttccagcctacaggagagccaacgatcctctacgagcgtcaccagatgtaccgacagctccaggccaaagggctcccaacggaaggtcatcccccagacctggtaaataaggtagctggtgggtatggaaaatacagcgagcaacacgctaaactggcccgtgccgtaaagatcgacagggacagcgccctggagtcctgctcctgggggatgttccagatcatgggctaccactggaagctgatggggtaccctacccttcaagctttcgtaaacgccatgtacgccagcgaaggagcccagatggacgccttctgccggttcatcaaggcacaacccaccacgcatgctgccttgaaagcccatgattgggccaagtttgccagactgtacaacggtccaggctacgccaagaacaagtatgacgtgaaattggagaaagcatatgctgaagctagtggctgataaaagcttggctgttttggc

SEQ ID NO: 26 - protein, 187 aa, Pseudomonas phage KPP10
MITDREYQQAAEMLGVDVPAIKAVTKVEAPVGGFQPTGEPTILYERHQMYRQLQAKGLPTEGHPPDLVNKVAGGYGKYSEQHAKLARAVKIDRDSALESCSWGMFQIMGYHWKLMGYPTLQAFVNAMYASEGAQMDAFCRFIKAQPTTHAALKAHDWAKFARLYNGPGYAKNKYDVKLEKAYAEASG

SEQ ID NO: 27 - DNA, 609 bp, Delftia sp.
  CDS (28)..(585)
  misc_feature (28)..(585): GN333 lysin
gtttaactttaagaaggagaattcaccatggctctaactgagcaagacttccaatcggctgccgatgatctcggagtcgatgttgccagtgtaaaggccgtcactaaagtagagagtcgtgggagcggctttctactttctggcgtccctaagattctattcgaaaggcactggatgttcaagcttctcaaaaggaagctaggtcgtgaccctgaaataaacgacgtttgcaaccctaaagctggaggatacctcggcggacaagcggagcacgaacgtctagataaagcagtcaagatggatagagactgcgcacttcaaagtgcctcttggggcctattccagattatgggattccattgggaggcactaggttatgcgagtgttcaggcatttgtcaatgcccagtacgctagcgagggatcgcaactaaacacttttgtgcgcttcatcaagaccaacccggcaattcacaaagctttaaagtctaaggactgggcagaattcgcaagaaggtataacgggccggattacaagaaaaacaactacgatgttaagctagcagaagcctatcaatccttcaagtaataaaagcttggctgttttggc

SEQ ID NO: 28 - protein, 186 aa, Delftia sp.
MALTEQDFQSAADDLGVDVASVKAVTKVESRGSGFLLSGVPKILFERHWMFKLLKRKLGRDPEINDVCNPKAGGYLGGQAEHERLDKAVKMDRDCALQSASWGLFQIMGFHWEALGYASVQAFVNAQYASEGSQLNTFVRFIKTNPAIHKALKSKDWAEFARRYNGPDYKKNNYDVKLAEAYQSFK

SEQ ID NO: 29 - DNA, 984 bp, Artificial Sequence
  Description of Artificial Sequence Synthetic polynucleotide
  misc_feature (28)..(957): GN349 lysin
  CDS (28)..(957)
gtttaactttaagaaggagaattcaccatggccattttaaagattggcagcaaaggtctggaagttaagaatcttcagaccagtctcaacaaaatcgggttcaatctggttgccgatggcatatttggtaaagcgactgacaacgccgtcagggcagttcaggcaggtgccggactggtcgttgatggtattgctggccccaagaccatgtatgcgattcgcaacgcaggggagtctcatcaggatcatctgactgaggctgacttgattgacgctgctcgtgaattgtctgttgaccttgctagcatcaaggcagtcaaccaagtagaatcgcgcggtactggcttcaccaagtctggtaagatcaagacattgtttgaacgccacatcatgtacaaaaagctgaatgccaagttcggtcaggcaaaagccaatgctctggcccagctttacccgacgttggttaacgccaaagccgggggatacacaggtggggacgcggagttggaacgactccatggtgcaatagcgatcgataaagattgcgcctacgagagcgcttcctacgggttattccagatcatggggttcaactgcgttatttgtggatatgacaatgccgaggagatgttcaacgactttctcactggtgaacgtgctcagctcatggcatttgtcaagttcatcaaggctgacgccaatctgtggaaagcattgaaggacaagaattgggctgagtttgctcggcgttacaatggcccggcgtatgcacagaaccagtacgacaccaagctggctgcagcatacaaatcattcagtaccgcgggcggcaccgcgggcggcgcacgaagatacagactttcgcgacgcagaagtcgacgacttttttcaagaactgcattaagaatgcatcgaagaaatagacttcgaagaattatgcgtggcggcattaggttttagtaataaaagcttggctgttttggc

SEQ ID NO: 30 - protein, 310 aa, Artificial Sequence
  Synthetic Construct
MAILKIGSKGLEVKNLQTSLNKIGFNLVADGIFGKATDNAVRAVQAGAGLVVDGIAGPKTMYAIRNAGESHQDHLTEADLIDAARELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVKFIKADANLWKALKDKNWAEFARRYNGPAYAQNQYDTKLAAAYKSFSTAGGTAGGARRYRLSRRRSRRLFSRTALRMHRRNRLRRIMRGGIRF

SEQ ID NO: 31 - DNA, 984 bp, Artificial Sequence
  Description of Artificial Sequence Synthetic polynucleotide
  misc_feature (28)..(957): GN351 lysin
  CDS (28)..(957)
gtttaactttaagaaggagaattcaccatggccattttaaagattggcagcaaaggtctggaagttaagaatcttcagaccagtctcaacaaaatcgggttcaatctggttgccgatggcatatttggtaaagcgactgacaacgccgtcagggcagttcaggcaggtgccggactggtcgttgatggtattgctggccccaagaccatgtatgcgattcgcaacgcaggggagtctcatcaggatcatctgactgaggctgacttgattgacgctgctcgtgaattgtctgttgaccttgctagcatcaaggcagtcaaccaagtagaatcgcgcggtactggcttcaccaagtctggtaagatcaagacattgtttgaacgccacatcatgtacaaaaagctgaatgccaagttcggtcaggcaaaagccaatgctctggcccagctttacccgacgttggttaacgccaaagccgggggatacacaggtggggacgcggagttggaacgactccatggtgcaatagcgatcgataaagattgcgcctacgagagcgcttcctacgggttattccagatcatggggttcaactgcgttatttgtggatatgacaatgccgaggagatgttcaacgactttctcactggtgaacgtgctcagctcatggcatttgtcaagttcatcaaggctgacgccaatctgtggaaagcattgaaggacaagaattgggctgagtttgctcggcgttacaatggcccggcgtatgcacagaaccagtacgacaccaagctggctgcagcatacaaatcattcagtaccgcgggcggcaccgcgggcggcgctcgttcccgtagacgtatgtctaagcgttcttcccgccgttcgttccgcaagtatgcgaagtcgcataagaagaactttaaagcccgctcaatgcgtggcggtatccgtttatgataataaaagcttggctgttttggc

SEQ ID NO: 32 - protein, 310 aa, Artificial Sequence
  Synthetic Construct
MAILKIGSKGLEVKNLQTSLNKIGFNLVADGIFGKATDNAVRAVQAGAGLVVDGIAGPKTMYAIRNAGESHQDHLTEADLIDAARELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVKFIKADANLWKALKDKNWAEFARRYNGPAYAQNQYDTKLAAAYKSFSTAGGTAGGARSRRRMSKRSSRRSFRKYAKSHKKNFKARSMRGGIRL

SEQ ID NO: 33 - DNA, 981 bp, Artificial Sequence
  Description of Artificial Sequence Synthetic polynucleotide
  misc_feature: GN352 lysin28
  CDS (28)..(954)
gtttaactttaagaaggagaattcaccatggccattttaaagattggcagcaaaggtctggaagttaagaatcttcagaccagtctcaacaaaatcgggttcaatctggttgccgatggcatatttggtaaagcgactgacaacgccgtcagggcagttcaggcaggtgccggactggtcgttgatggtattgctggccccaagaccatgtatgcgattcgcaacgcaggggagtctcatcaggatcatctgactgaggctgacttgattgacgctgctcgtgaattgtctgttgaccttgctagcatcaaggcagtcaaccaagtagaatcgcgcggtactggcttcaccaagtctggtaagatcaagacattgtttgaacgccacatcatgtacaaaaagctgaatgccaagttcggtcaggcaaaagccaatgctctggcccagctttacccgacgttggttaacgccaaagccgggggatacacaggtggggacgcggagttggaacgactccatggtgcaatagcgatcgataaagattgcgcctacgagagcgcttcctacgggttattccagatcatggggttcaactgcgttatttgtggatatgacaatgccgaggagatgttcaacgactttctcactggtgaacgtgctcagctcatggcatttgtcaagttcatcaaggctgacgccaatctgtggaaagcattgaaggacaagaattgggctgagtttgctcggcgttacaatggcccggcgtatgcacagaaccagtacgacaccaagctggctgcagcatacaaatcattcagtaccgcgggcggcaccgcgggcggcaaacgtagaaaaatgacaagaaaaggttctaagcgtctttttactgcaactgctgataaaactaaatctatcaatactgccccgccgccaatgcgtggcggtatccggttgtagtaataaaagcttggctgttttggc

SEQ ID NO: 34 - protein, 309 aa, Artificial Sequence
  Synthetic Construct
MAILKIGSKGLEVKNLQTSLNKIGFNLVADGIFGKATDNAVRAVQAGAGLVVDGIAGPKTMYAIRNAGESHQDHLTEADLIDAARELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVKFIKADANLWKALKDKNWAEFARRYNGPAYAQNQYDTKLAAAYKSFSTAGGTAGGKRRKMTRKGSKRLFTATADKTKSINTAPPPMRGGIRL

SEQ ID NO: 35 - DNA, 978 bp, Artificial Sequence
  Description of Artificial Sequence Synthetic polynucleotide
  misc_feature (28)..(951): GN353 lysin
  CDS (28)..(951)
gtttaactttaagaaggagaattcaccatggccattttaaagattggcagcaaaggtctggaagttaagaatcttcagaccagtctcaacaaaatcgggttcaatctggttgccgatggcatatttggtaaagcgactgacaacgccgtcagggcagttcaggcaggtgccggactggtcgttgatggtattgctggccccaagaccatgtatgcgattcgcaacgcaggggagtctcatcaggatcatctgactgaggctgacttgattgacgctgctcgtgaattgtctgttgaccttgctagcatcaaggcagtcaaccaagtagaatcgcgcggtactggcttcaccaagtctggtaagatcaagacattgtttgaacgccacatcatgtacaaaaagctgaatgccaagttcggtcaggcaaaagccaatgctctggcccagctttacccgacgttggttaacgccaaagccgggggatacacaggtggggacgcggagttggaacgactccatggtgcaatagcgatcgataaagattgcgcctacgagagcgcttcctacgggttattccagatcatggggttcaactgcgttatttgtggatatgacaatgccgaggagatgttcaacgactttctcactggtgaacgtgctcagctcatggcatttgtcaagttcatcaaggctgacgccaatctgtggaaagcattgaaggacaagaattgggctgagtttgctcggcgttacaatggcccggcgtatgcacagaaccagtacgacaccaagctggctgcagcatacaaatcattcagtaccgcgggcggcaccgcgggcggcagaaagcgaatgtctaagcgtgttgacaagaaggtgttccgtcgtactgccgcatctgccaagaagattaacattgaccccaagatttaccgtggaggtattcgcctatgataataaaagcttggctgttttggc

SEQ ID NO: 36 - protein, 308 aa, Artificial Sequence
  Synthetic Construct
MAILKIGSKGLEVKNLQTSLNKIGFNLVADGIFGKATDNAVRAVQAGAGLVVDGIAGPKTMYAIRNAGESHQDHLTEADLIDAARELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVKFIKADANLWKALKDKNWAEFARRYNGPAYAQNQYDTKLAAAYKSFSTAGGTAGGRKRMSKRVDKKVFRRTAASAKKINIDPKIYRGGIRL

SEQ ID NO: 37 - DNA, 903 bp, Artificial Sequence
  Description of Artificial Sequence Synthetic polynucleotide
  misc_feature (28)..(879)
  misc_feature (28)..(879): GN357 lysin
  CDS (28)..(879)
gtttaactttaagaaggagaattcaccatggccattttaaagattggcagcaaaggtctggaagttaagaatcttcagaccagtctcaacaaaatcgggttcaatctggttgccgatggcatatttggtaaagcgactgacaacgccgtcagggcagttcaggcaggtgccggactggtcgttgatggtattgctggccccaagaccatgtatgcgattcgcaacgcaggggagtctcatcaggatcatctgactgaggctgacttgattgacgctgctcgtgaattgtctgttgaccttgctagcatcaaggcagtcaaccaagtagaatcgcgcggtactggcttcaccaagtctggtaagatcaagacattgtttgaacgccacatcatgtacaaaaagctgaatgccaagttcggtcaggcaaaagccaatgctctggcccagctttacccgacgttggttaacgccaaagccgggggatacacaggtggggacgcggagttggaacgactccatggtgcaatagcgatcgataaagattgcgcctacgagagcgcttcctacgggttattccagatcatggggttcaactgcgttatttgtggatatgacaatgccgaggagatgttcaacgactttctcactggtgaacgtgctcagctcatggcatttgtcaagttcatcaaggctgacgccaatctgtggaaagcattgaaggacaagaattgggctgagtttgctcggcgttacaatggcccggcgtatgcacagaaccagtacgacaccaagctggctgcagcatacaaatcattcagtaccgcgggcggcaccgcgggcggccgccgcctgattcgcctgtggctgcgcctgctgcgctaataaaagcttggctgttttggc

SEQ ID NO: 38 - protein, 284 aa, Artificial Sequence
  Synthetic Construct
MAILKIGSKGLEVKNLQTSLNKIGFNLVADGIFGKATDNAVRAVQAGAGLVVDGIAGPKTMYAIRNAGESHQDHLTEADLIDAARELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVKFIKADANLWKALKDKNWAEFARRYNGPAYAQNQYDTKLAAAYKSFSTAGGTAGGRRLIRLWLRLLR

SEQ ID NO: 39 - DNA, 912 bp, Artificial Sequence
  Description of Artificial Sequence Synthetic polynucleotide
  misc_feature (28)..(888): GN359 lysin
  CDS (28)..(888)
gtttaactttaagaaggagaattcaccatggccattttaaagattggcagcaaaggtctggaagttaagaatcttcagaccagtctcaacaaaatcgggttcaatctggttgccgatggcatatttggtaaagcgactgacaacgccgtcagggcagttcaggcaggtgccggactggtcgttgatggtattgctggccccaagaccatgtatgcgattcgcaacgcaggggagtctcatcaggatcatctgactgaggctgacttgattgacgctgctcgtgaattgtctgttgaccttgctagcatcaaggcagtcaaccaagtagaatcgcgcggtactggcttcaccaagtctggtaagatcaagacattgtttgaacgccacatcatgtacaaaaagctgaatgccaagttcggtcaggcaaaagccaatgctctggcccagctttacccgacgttggttaacgccaaagccgggggatacacaggtggggacgcggagttggaacgactccatggtgcaatagcgatcgataaagattgcgcctacgagagcgcttcctacgggttattccagatcatggggttcaactgcgttatttgtggatatgacaatgccgaggagatgttcaacgactttctcactggtgaacgtgctcagctcatggcatttgtcaagttcatcaaggctgacgccaatctgtggaaagcattgaaggacaagaattgggctgagtttgctcggcgttacaatggcccggcgtatgcacagaaccagtacgacaccaagctggctgcagcatacaaatcattcagtaccgcgggcggcaccgcgggcggcacccgcaaacgcctgaaaaaaattggcaaagtgctgaaatggatttaataaaagcttggctgttttggc

SEQ ID NO: 40 - protein, 287 aa, Artificial Sequence
  Synthetic Construct
MAILKIGSKGLEVKNLQTSLNKIGFNLVADGIFGKATDNAVRAVQAGAGLVVDGIAGPKTMYAIRNAGESHQDHLTEADLIDAARELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVKFIKADANLWKALKDKNWAEFARRYNGPAYAQNQYDTKLAAAYKSFSTAGGTAGGTRKRLKKIGKVLKWI

SEQ ID NO: 41 - DNA, 897 bp, Artificial Sequence
  Description of Artificial Sequence Synthetic polynucleotide
  misc_feature (28)..(873): GN369 lysin
  CDS (28)..(873)
gtttaactttaagaaggagaattcaccatggccattttaaagattggcagcaaaggtctggaagttaagaatcttcagaccagtctcaacaaaatcgggttcaatctggttgccgatggcatatttggtaaagcgactgacaacgccgtcagggcagttcaggcaggtgccggactggtcgttgatggtattgctggccccaagaccatgtatgcgattcgcaacgcaggggagtctcatcaggatcatctgactgaggctgacttgattgacgctgctcgtgaattgtctgttgaccttgctagcatcaaggcagtcaaccaagtagaatcgcgcggtactggcttcaccaagtctggtaagatcaagacattgtttgaacgccacatcatgtacaaaaagctgaatgccaagttcggtcaggcaaaagccaatgctctggcccagctttacccgacgttggttaacgccaaagccgggggatacacaggtggggacgcggagttggaacgactccatggtgcaatagcgatcgataaagattgcgcctacgagagcgcttcctacgggttattccagatcatggggttcaactgcgttatttgtggatatgacaatgccgaggagatgttcaacgactttctcactggtgaacgtgctcagctcatggcatttgtcaagttcatcaaggctgacgccaatctgtggaaagcattgaaggacaagaattgggctgagtttgctcggcgttacaatggcccggcgtatgcacagaaccagtacgacaccaagctggctgcagcatacaaatcattcagtcgtaaaaaaacccgtaaacgtctgaaaaaaatcggtaaagttctgaaatggatctagtaaaagcttggctgttttggc

SEQ ID NO: 42 - protein, 282 aa, Artificial Sequence
  Synthetic Construct
MAILKIGSKGLEVKNLQTSLNKIGFNLVADGIFGKATDNAVRAVQAGAGLVVDGIAGPKTMYAIRNAGESHQDHLTEADLIDAARELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVKFIKADANLWKALKDKNWAEFARRYNGPAYAQNQYDTKLAAAYKSFSRKKTRKRLKKIGKVLKWI

SEQ ID NO: 43 - DNA, 558 bp, Artificial Sequence
  Description of Artificial Sequence Synthetic polynucleotide
  misc_feature (28)..(534): GN370 lysin
  CDS (28)..(534)
gtttaactttaagaaggagaattcaccatgatcgaccgtttcattcgtctgaatccgacccatggtccgcgtcgtccgcgtcgtccgggtcgtcgtgctccggttcgtacatcccaacgaggcatcgacctcatcaaatccttcgagggcctgcgcctgtccgcttaccaggactcggtgggtgtctggaccataggttacggcaccactcggggcgtcacccgctacatgacgatcaccgtcgagcaggccgagcggatgctgtcgaacgacattcagcgcttcgagccagagctagacaggctggcgaaggtgccactgaaccagaaccagtgggatgccctgatgagcttcgtgtacaacctgggcgcggccaatctggcgtcgtccacgctgctcgacctgctgaacaagggtgactaccagggagcagcggaccagttcccgcattgggtgaatgcgggcggtaagcgcttggatggtctggttaagcgtcgagcagccgagcgtgcgctgttcctggagccactatcgtgataaaagcttggctgttttggc

SEQ ID NO: 44 - protein, 169 aa, Artificial Sequence
  Synthetic Construct
MIDRFIRLNPTHGPRRPRRPGRRAPVRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLDLLNKGDYQGAADQFPHWVNAGGKRLDGLVKRRAAERALFLEPLS

SEQ ID NO: 45 - DNA, 516 bp, Artificial Sequence
  Description of Artificial Sequence Synthetic polynucleotide
  misc_feature (28)..(492): GN371lysin
  CDS (28)..(492)
gtttaactttaagaaggagaattcaccatgatcgaccgtttcattcgtctgaatccgacccatcgtacatcccaacgaggcatcgacctcatcaaatccttcgagggcctgcgcctgtccgcttaccaggactcggtgggtgtctggaccataggttacggcaccactcggggcgtcacccgctacatgacgatcaccgtcgagcaggccgagcggatgctgtcgaacgacattcagcgcttcgagccagagctagacaggctggcgaaggtgccactgaaccagaaccagtgggatgccctgatgagcttcgtgtacaacctgggcgcggccaatctggcgtcgtccacgctgctcgacctgctgaacaagggtgactaccagggagcagcggaccagttcccgcattgggtgaatgcgggcggtaagcgcttggatggtctggttaagcgtcgagcagccgagcgtgcgctgttcctggagccactatcgtgataaaagcttggctgttttggc

SEQ ID NO: 46 - protein, 155 aa, Artificial Sequence
  Synthetic Construct
MIDRFIRLNPTHRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLDLLNKGDYQGAADQFPHWVNAGGKRLDGLVKRRAAERALFLEPLS

SEQ ID NO: 47 - DNA, 846 bp, Artificial Sequence
  Description of Artificial Sequence Synthetic polynucleotide
  misc_feature (28)..(819): GN394 lysin
  CDS (28)..(819)
gtttaactttaagaaggagaattcaccatggccattttaaagattggcagcaaaggtctggaagttaagaatcttcagaccagtctcaacaaaatcgggttcaatctggttgccgatggcatatttggtaaagcgactgacaacgccgtcagggcagttcaggcaggtgccggactggtcgttgatggtattgctggccccaagaccatgtatgcgattcgcaacgcaggggagtctcatcaggatcatctgactgaggctgacttgattgacgctgctcgtgaattgtctgttgaccttgctagcatcaaggcagtcaaccaagtagaatcgcgcggtactggcttcaccaagtctggtaagatcaagacattgtttgaacgccacatcatgtacaaaaagctgaatgccaagttcggtcaggcaaaagccaatgctctggcccagctttacccgacgttggttaacgccaaagccgggggatacacaggtggggacgcggagttggaacgactccatggtgcaatagcgatcgataaagattgcgcctacgagagcgcttcctacgggttattccagatcatggggttcaactgcgttatttgtggatatgacaatgccgaggagatgttcaacgactttctcactggtgaacgtgctcagctcatggcatttgtcgacttcatcaaggctgacgccaatctgtggaaagcattgaaggacaagaattgggctgagtttgctcggcgttacaatggcccggcgtatgcacagaaccagtacgacaccaagctggctgcagcatacaaatcattcagttagtaataaaagcttggctgttttggc

SEQ ID NO: 48 - protein, 264 aa, Artificial Sequence
  Synthetic Construct
MAILKIGSKGLEVKNLQTSLNKIGFNLVADGIFGKATDNAVRAVQAGAGLVVDGIAGPKTMYAIRNAGESHQDHLTEADLIDAARELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVDFIKADANLWKALKDKNWAEFARRYNGPAYAQNQYDTKLAAAYKSFS

SEQ ID NO: 49 - DNA, 846 bp, Artificial Sequence
  Description of Artificial Sequence Synthetic polynucleotide
  misc_feature (28)..(819): GN396 lysin
  CDS (28)..(819)
gtttaactttaagaaggagaattcaccatggccattttaaagattggcagcaaaggtctggaagttaagaatcttcagaccagtctcaacaaaatcgggttcaatctggttgccgatggcatatttggtaaagcgactgacaacgccgtcagggcagttcaggcaggtgccggactggtcgttgatggtattgctggccccaagaccatgtatgcgattcgcaacgcaggggagtctcatcaggatcatctgactgaggctgacttgattgacgctgctcgtgaattgtctgttgaccttgctagcatcaaggcagtcaaccaagtagaatcgcgcggtactggcttcaccaagtctggtaagatcaagacattgtttgaacgccacatcatgtacaaaaagctgaatgccaagttcggtcaggcaaaagccaatgctctggcccagctttacccgacgttggttaacgccaaagccgggggatacacaggtggggacgcggagttggaacgactccatggtgcaatagcgatcgataaagattgcgcctacgagagcgcttcctacgggttattccagatcatggggttcaactgcgttatttgtggatatgacaatgccgaggagatgttcaacgactttctcactggtgaacgtgctcagctcatggcatttgtcaagttcatcaaggctgacgccaatctgtgggacgcattgaaggacaagaattgggctgagtttgctcggcgttacaatggcccggcgtatgcacagaaccagtacgacaccaagctggctgcagcatacaaatcattcagttagtaataaaagcttggctgttttggc

SEQ ID NO: 50 - protein, 264 aa, Artificial Sequence
  Synthetic Construct
MAILKIGSKGLEVKNLQTSLNKIGFNLVADGIFGKATDNAVRAVQAGAGLVVDGIAGPKTMYAIRNAGESHQDHLTEADLIDAARELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVKFIKADANLWDALKDKNWAEFARRYNGPAYAQNQYDTKLAAAYKSFS

SEQ ID NO: 51 - DNA, 846 bp, Artificial Sequence
  Description of Artificial Sequence Synthetic polynucleotide
  misc_feature (28)..(819): GN408 lysin
  CDS (28)..(819)
gtttaactttaagaaggagaattcaccatggccattttaaagattggcagcaaaggtctggaagttaagaatcttcagaccagtctcaacaaaatcgggttcaatctggttgccgatggcatatttggtaaagcgactgacaacgccgtcagggcagttcaggcaggtgccggactggtcgttgatggtattgctggccccaagaccatgtatgcgattcgcaacgcaggggagtctcatcaggatcatctgactgaggctgacttgattgacgctgctcatgaattgtctgttgaccttgctagcatcaaggcagtcaaccaagtagaatcgcgcggtactggcttcaccaagtctggtaagatcaagacattgtttgaacgccacatcatgtacaaaaagctgaatgccaagttcggtcaggcaaaagccaatgctctggcccagctttacccgacgttggttaacgccaaagccgggggatacacaggtggggacgcggagttggaacgactccatggtgcaatagcgatcgataaagattgcgcctacgagagcgcttcctacgggttattccagatcatggggttcaactgcgttatttgtggatatgacaatgccgaggagatgttcaacgactttctcactggtgaacgtgctcagctcatggcatttgtcaagttcatcaaggctgacgccaatctgtggaaagcattgaaggacaagaattgggctgagtttgctcggcgttacaatggcccggcgtatgcacagaaccagtacgacaccaagctggctgcagcatacaaatcattcagttagtaataaaagcttggctgttttggc

SEQ ID NO: 52 - protein, 264 aa, Artificial Sequence
  Synthetic Construct
MAILKIGSKGLEVKNLQTSLNKIGFNLVADGIFGKATDNAVRAVQAGAGLVVDGIAGPKTMYAIRNAGESHQDHLTEADLIDAAHELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVKFIKADANLWKALKDKNWAEFARRYNGPAYAQNQYDTKLAAAYKSFS

SEQ ID NO: 53 - DNA, 846 bp, Artificial Sequence
  Description of Artificial Sequence Synthetic polynucleotide
  misc_feature (28)..(819): GN418 lysin
  CDS (28)..(819)
gtttaactttaagaaggagaattcaccatggccattttaaagattggcagcaaaggtctggaagttaagaatcttcagaccagtctcaacgacatcgggttcaatctggttgccgatggcatatttggtaaagcgactgacaacgccgtcagggcagttcaggcaggtgccggactggtcgttgatggtattgctggccccaagaccatgtatgcgattcgcaacgcaggggagtctcatcaggatcatctgactgaggctgacttgattgacgctgctcgtgaattgtctgttgaccttgctagcatcaaggcagtcaaccaagtagaatcgcgcggtactggcttcaccaagtctggtaagatcaagacattgtttgaacgccacatcatgtacaaaaagctgaatgccaagttcggtcaggcaaaagccaatgctctggcccagctttacccgacgttggttaacgccaaagccgggggatacacaggtggggacgcggagttggaacgactccatggtgcaatagcgatcgataaagattgcgcctacgagagcgcttcctacgggttattccagatcatggggttcaactgcgttatttgtggatatgacaatgccgaggagatgttcaacgactttctcactggtgaacgtgctcagctcatggcatttgtcaagttcatcaaggctgacgccaatctgtggaaagcattgaaggacaagaattgggctgagtttgctcggcgttacaatggcccggcgtatgcacagaaccagtacgacaccaagctggctgcagcatacaaatcattcagttagtaataaaagcttggctgttttggc

SEQ ID NO: 54 - protein, 264 aa, Artificial Sequence
  Synthetic Construct
MAILKIGSKGLEVKNLQTSLNDIGFNLVADGIFGKATDNAVRAVQAGAGLVVDGIAGPKTMYAIRNAGESHQDHLTEADLIDAARELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVKFIKADANLWKALKDKNWAEFARRYNGPAYAQNQYDTKLAAAYKSFS

SEQ ID NO: 55 - DNA, 858 bp, Burkholderia pseudomultivorans
  misc_feature (28)..(834): GN424 lysin
  CDS (28)..(834)
gtttaactttaagaaggagaattcaccatgaatacccttcgtttcaacagtcgcggcgccgaagtcggcgtgctgcagcaacggctcgtgcgcgccggctatccgatcgacgtcacgcatctctatgacgaagcgacggagcaggccgtgaaggcgttgcaggcagcggccggaatcgtcgtcgacggaatcgccggcccgaacacctatgccgtgttgtcggccggccagcgcgaccgcaagcacttgaccgaagcggacatcgcccgcgccgcagacaagctcggtgtctcgccggcatgcgtccgcgccgtcaacgaagtcgagtcacgcggctcgggctttctggcggacggccggcccgtgattctcttcgagcggcacgtgatgtacaaccgcctcgtcgcggcgaagcgtgccgtcgacgcagcgagcgcagcgcagcgctttccgaacgtcgtcagcgcgaagccgggcggataccagggcggcgcagccgaatatgtgcgactcgacaccgccgcgcgcatcgatgcggcaatcgcgtacgaatcggcgagctggggcgcatttcaggtgatgggctatcactgggaacgcctgggctactcgagcatcgacgagttcgttgcccggatggagacgagcgaaggcgaacagctcgacgcgtttgtgcggttcgtcgccgccgactcgtcgctgcgcacggcgctgaaaaaccggaagtgggctgcattcgcgaagggctacaacggcccggactatgcgcgcaacctctacgacgcgaagctcgcccaggcgtacgaacggtatgccggcacgaaggcggccgcgtgataaaagcttggctgttttggc

SEQ ID NO: 56 - protein, 269 aa, Burkholderia pseudomultivorans
MNTLRFNSRGAEVGVLQQRLVRAGYPIDVTHLYDEATEQAVKALQAAAGIVVDGIAGPNTYAVLSAGQRDRKHLTEADIARAADKLGVSPACVRAVNEVESRGSGFLADGRPVILFERHVMYNRLVAAKRAVDAASAAQRFPNVVSAKPGGYQGGAAEYVRLDTAARIDAAIAYESASWGAFQVMGYHWERLGYSSIDEFVARMETSEGEQLDAFVRFVAADSSLRTALKNRKWAAFAKGYNGPDYARNLYDAKLAQAYERYAGTKAAA

SEQ ID NO: 57 - DNA, 864 bp, Pseudomonas flexibilis
  misc_feature (28)..(840): GN425 lysin
  CDS (28)..(840)
gtttaactttaagaaggagaattcaccatgaccctgcgcctcgatgacgtcggcctcgacgtgctccatctgcagaagcgcctcaacgagctgggcgcgaatccgcggctgctgcccgatggccagttcggcgaggtcaccgagcgcgccgtgcgggccttccagcaacgtgccggcctggtggtcgatggcgtggccggacccaagacgatggccgccctgtccggccattccaccagccgcctgctcggccagcgcgacctgcaacgcgccgccgaccgcttgggcgtgccgctggccagcgtcatggccctcaatgccgtggaaagtcgcggcgagggcttcgccgccaatggccggccggtgatcctgttcgagcggcacgtgatgcacgaacgcttgcaggtcaacggcctgagcgaagccgaggcggacgccctggcggcacgccaccccggcctggtgagtcgccggccaggcggctacgtcggcgacaccgccgagcatcagcgcctggccaatgcccgcctgttgcatgacaccgctgccctggaatccgccagttggggactgttccaggtgatgggctaccactggcaggccctgggctacgacaccacccaggacttcaccgagcgcatggcccgccacgaagccgagcacctggaagcgttcgtgcgcttcatcgaagccgatccggcactgcacaaggcactcaagggccgtaagtgggccgagttcgcccgccgctacaacggcccggcctacgcccgcaatttgtacgacgtgaagctggctcgggcattcgagcaattcagcgacgcactgcaggccgccgcatgataaaagcttggctgttttggc

SEQ ID NO: 58 - protein, 271 aa, Pseudomonas flexibilis
MTLRLDDVGLDVLHLQKRLNELGANPRLLPDGQFGEVTERAVRAFQQRAGLVVDGVAGPKTMAALSGHSTSRLLGQRDLQRAADRLGVPLASVMALNAVESRGEGFAANGRPVILFERHVMHERLQVNGLSEAEADALAARHPGLVSRRPGGYVGDTAEHQRLANARLLHDTAALESASWGLFQVMGYHWQALGYDTTQDFTERMARHEAEHLEAFVRFIEADPALHKALKGRKWAEFARRYNGPAYARNLYDVKLARAFEQFSDALQAAA

SEQ ID NO: 59 - DNA, 843 bp, Escherichia virus
  misc_feature (28)..(819): GN428 lysin
  CDS (28)..(819)
gtttaactttaagaaggagaattcaccatggccattctaaaacttggcaaccgaggttctgaagtcaaagcacttcaacaaagcctcaacaaaatcggtttctctcttacagccgatggcatatttggtaaggcaacagagaatgccgtcaaatccgttcaggcaggtgctggattggttattgatggtattgctgggccaaagaccttctatgctatccgcaacgctggagacgctcaccaggaacatctgaccgaagcggacttggttgacgcagcacgtgaacttggtgttgagctggccagtatgaaagcggtgaaccaggtagaatcccgtggtacgggttttaccaaaactggcaagatcaaaactctgtttgagcgccacatcatgtacaaaaaggtgacggccaaattcgggcaagcaagagccaatgctctgtaccaactctacccaacattggttaaccccaattctggcgggtatatcggcggagacgcggagttggaacgccttcagggtgcaatcgcccttgacgaggactgcgcttacgagagtgcttcctacggcctattccagatcatggggttcaactgccaaatctgtggctattcaaatgccaaagagatgttcactgatttcctgactggtgaacgcgctcatcttctggcatttgtcaagttcatcaaggctgatgccaatatgtggaaagccctgaagaacaagaattgggccgagtttgctcgtcggtacaatggtccggcatatgcgaaaaaccagtatgatactaaactggcggcagcatacaagagtttctgttaataaaagcttggctgttttggc

SEQ ID NO: 60 - protein, 264 aa, Escherichia virus
MAILKLGNRGSEVKALQQSLNKIGFSLTADGIFGKATENAVKSVQAGAGLVIDGIAGPKTFYAIRNAGDAHQEHLTEADLVDAARELGVELASMKAVNQVESRGTGFTKTGKIKTLFERHIMYKKVTAKFGQARANALYQLYPTLVNPNSGGYIGGDAELERLQGAIALDEDCAYESASYGLFQIMGFNCQICGYSNAKEMFTDFLTGERAHLLAFVKFIKADANMWKALKNKNWAEFARRYNGPAYAKNQYDTKLAAAYKSFC

SEQ ID NO: 61 - DNA, 660 bp, Artificial Sequence
  Description of Artificial Sequence Synthetic polynucleotide
  misc_feature (13)..(639): GN93 lysin
  CDS (13)..(639)
ggagaattcaccatgaaattctttaagttctttaagttttttaaagccggcgcaggagctggtgcaggagctggtgcaggagctggtgcaggagctagcaataacgaacttccttgggtagccgaagcccgaaagtatatcggccttcgcgaagacacttcgaagacttcgcataacccgaaacttcttgccatgcttgaccgcatgggcgaattttccaacgaatcccgcgcttggtggcacgacgacgaaacgccttggtgcggactgttcgtcggctattgcttgggcgttgccgggcgctacgtcgtccgcgaatggtacagggcgcgggcatgggaagccccgcagcttacgaagcttgaccggcccgcatacggcgcgcttgtgaccttcacgcgaagcggcggcggccacgtcggttttattgtgggcaaggatgcgcgcggaaatcttatggttcttggcggtaatcagtcgaacgccgtaagtatcgcaccgttcgcagtatcccgcgtaaccggctatttctggccgtcgttctggcgaaacaagaccgcagttaaaagcgttccgtttgaagaacgttattcgctgccgctgttgaagtcgaacggcgaactttcgacgaatgaagcgtaataagcttggctgttttgg

SEQ ID NO: 62 - protein, 209 aa, Artificial Sequence
  Synthetic Construct
MKFFKFFKFFKAGAGAGAGAGAGAGAGASNNELPWVAEARKYIGLREDTSKTSHNPKLLAMLDRMGEFSNESRAWWHDDETPWCGLFVGYCLGVAGRYVVREWYRARAWEAPQLTKLDRPAYGALVTFTRSGGGHVGFIVGKDARGNLMVLGGNQSNAVSIAPFAVSRVTGYFWPSFWRNKTAVKSVPFEERYSLPLLKSNGELSTNEA

SEQ ID NO: 63 - DNA, 843 bp, Dickeya phage phiD3
  misc_feature (28)..(819): GN431 lysin
  CDS (28)..(819)
gtttaactttaagaaggagaattcaccatggccattctaaaacttggcaaccgtggcactgaagtgaaggcacttcaggatagcctcaacaaaatcggcttcaccctcgtcgctgacggcatctttggtaaggcaacagagaacgctgtcaagaccgttcaggcgggtgcggggcttgtcattgatggtatcgtgggtccaaagacctcctatgctattcgcaacgccggggaagcgcatcaggatcacctgactgaggctgaccttatcgaggcggccaatcagctgggcgtcgacctcgcttctgtgaaggcagtcaaccaggttgaatcccgtggcacaggcttcaccaagtcaggcaagatcaagacattgttcgagcgtcacatcatgtataagaaactgatggcaaagttcggacaggctcgagcgaatgccatgggtcagatgtatccgactctggtcagcccggttgcaggcgggtacacgggaggtgacgcagaattggatcgactccacgcagcgatcaacatcgacgaggattgtgcgtacgagagcgcttcatacggcctcttccagatcatgggcttcaactgccaggtctgcgggtatgccaacgccaaggagatgttcaatgacttcctgacgggagaacgtgctcacctgatggcattcgtgaagttcatcaaggctgatgccaagctctggcaggctctgaaggacaagaattgggctgagttcgcgcggcgctataatggtccggcgtataccaagaaccagtacgacacgaagctcgcagcagcatacaacagcttcaattaataaaagcttggctgttttggc

SEQ ID NO: 64 - protein, 264 aa, Dickeya phage phiD3
MAILKLGNRGTEVKALQDSLNKIGFTLVADGIFGKATENAVKTVQAGAGLVIDGIVGPKTSYAIRNAGEAHQDHLTEADLIEAANQLGVDLASVKAVNQVESRGTGFTKSGKIKTLFERHIMYKKLMAKFGQARANAMGQMYPTLVSPVAGGYTGGDAELDRLHAAINIDEDCAYESASYGLFQIMGFNCQVCGYANAKEMFNDFLTGERAHLMAFVKFIKADAKLWQALKDKNWAEFARRYNGPAYTKNQYDTKLAAAYNSFN

SEQ ID NO: 65 - DNA, 510 bp, Artificial Sequence
  Description of Artificial Sequence Synthetic polynucleotide
  misc_feature (10)..(510): GN486 lysin
  CDS (10)..(510)
gaattcaccatgggatcccatcatcaccaccatcatggtggtccgcgtcgtccgcgtcgtccgggtcgtcgtgctccggttcgtacctctcagcgtggtatcgacctgatcaaatctttcgaaggtctgcgtctgtctgcttaccaggactctgttggtgtttggaccatcggttacggtaccacccgtggtgttacccgttacatgaccatcaccgttgaacaggctgaacgtatgctgtctaacgacatccagcgtttcgaaccggaactggaccgtctggctaaagttccgctgaaccagaaccagtgggacgctctgatgtctttcgtttacaacctgggtgctgctaacctggcttcttctaccctgctgaaactgctgaacaaaggtgactaccagggtgctgctgaccagttcccgcgttgggttaacgctggtggtaaacgtctggacggtctggttaaacgtcgtgctgctgaacgtgctctgttcctggaaccgctgtct

SEQ ID NO: 66 - protein, 167 aa, Artificial Sequence
  Synthetic Construct
MGSHHHHHHGGPRRPRRPGRRAPVRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLKLLNKGDYQGAADQFPRWVNAGGKRLDGLVKRRAAERALFLEPLS

SEQ ID NO: 67 - DNA, 219 bp, Artificial Sequence
  Description of Artificial Sequence Synthetic polynucleotide
  misc_feature (1)..(216): GN485 lysin
  CDS (1)..(216)
atgccgggtctgtctggtttcatccgtaacgctgacaccccggttacctctctgggttctgctggtcacgttcacgttccggaaggtccgctgatccgtatcaacccggactgcctgctgggtaccccgttcaaattcttcaagttcttcaagttcttcaagttctttaagttctttaagtttttcaagttcttcaagaacgaatgcgttctgctgtaa

SEQ ID NO: 68 - protein, 72 aa, Artificial Sequence
  Synthetic Construct
MPGLSGFIRNADTPVTSLGSAGHVHVPEGPLIRINPDCLLGTPFKFFKFFKFFKFFKFFKFFKFFKNECVLL

SEQ ID NO: 69 - DNA, 132 bp, Chlamydia phage 2
atgaggttaaaaatggcacgaagaagatacagacttccgcgacgtagaagtcgaagacttttttcaagaactgcattgaggatgcatccaagaaataggcttcgaagaattatgcgtggcggcattaggttc

SEQ ID NO: 70 - protein, 44 aa, Chlamydia phage 2
MRLKMARRRYRLPRRRSRRLFSRTALRMHPRNRLRRIMRGGIRF

SEQ ID NO: 71 - DNA, 24 bp, Artificial Sequence
  Description of Artificial Sequence Synthetic oligonucleotide
  misc_feature (1)..(24): linker
accgcgggcggcaccgcgggcggc

SEQ ID NO: 72 - protein, 8 aa, Artificial Sequence
  Description of Artificial Sequence Synthetic peptide
  MISC_FEATURE (1)..(8): linker
TAGGTAGG

SEQ ID NO: 73 - DNA, 435 bp, Pseudomonas phage PAJU2
  misc_feature (1)..(435): GN4
atgcgtacatcccaacgaggcatcgacctcatcaaatccttcgagggcctgcgcctgtccgcttaccaggactcggtgggtgtctggaccataggttacggcaccactcggggcgtcacccgctacatgacgatcaccgtcgagcaggccgagcggatgctgtcgaacgacattcagcgcttcgagccagagctagacaggctggcgaaggtgccactgaaccagaaccagtgggatgccctgatgagcttcgtgtacaacctgggcgcggccaatctggcgtcgtccacgctgctcaagctgctgaacaagggtgactaccagggagcagcggaccagttcccgcgctgggtgaatgcgggcggtaagcgcttggatggtctggttaagcgtcgagcagccgagcgtgcgctgttcctggagccactatcgtga

SEQ ID NO: 74 - protein, 144 aa, Pseudomonas phage PAJU2
  MISC_FEATURE (1)..(144): GN4
MRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLKLLNKGDYQGAADQFPRWVNAGGKRLDGLVKRRAAERALFLEPLS

SEQ ID NO: 75 - DNA, 63 bp, Penaeus chinensis
atgagctttaacgtgaccccgaaatttaaacgctggcagctgtattttcgcggccgcatgtgg

SEQ ID NO: 76 - protein, 21 aa, Penaeus chinensis
MSFNVTPKFKRWQLYFRGRMW

SEQ ID NO: 77 - DNA, 438 bp, Artificial Sequence
  Description of Artificial Sequence Synthetic polynucleotide
  misc_feature (1)..(438): Modified GN4 lysin, GN146
atgcgtacatcccaacgaggcatcgacctcatcaaatccttcgagggcctgcgcctgtccgcttaccaggactcggtgggtgtctggaccataggttacggcaccactcggggcgtcacccgctacatgacgatcaccgtcgagcaggccgagcggatgctgtcgaacgacattcagcgcttcgagccagagctagacaggctggcgaaggtgccactgaaccagaaccagtgggatgccctgatgagcttcgtgtacaacctgggcgcggccaatctggcgtcgtccacgctgctcgacctgctgaacaagggtgactaccagggagcagcggaccagttcccgcattgggtgaatgcgggcggtaagcgcttggatggtctggttaagcgtcgagcagccgagcgtgcgctgttcctggagccactatcgtgataa

SEQ ID NO: 78 - protein, 144 aa, Artificial Sequence
  Description of Artificial Sequence Synthetic polypeptide
  MISC_FEATURE (1)..(144): Modified GN4 lysin, GN146
MRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLDLLNKGDYQGAADQFPHWVNAGGKRLDGLVKRRAAERALFLEPLS

SEQ ID NO: 79 - DNA, 57 bp, Pelophylax esculentus
atttttagcaaactggcgggcaaaaaaattaaaaacctgctgattagcggcctgaaa

SEQ ID NO: 80 - protein, 19 aa, Pelophylax esculentus
IFSKLAGKKIKNLLISGLK

SEQ ID NO: 81 - DNA, 36 bp, Artificial Sequence
  Description of Artificial Sequence Synthetic oligonucleotide
  misc_feature (1)..(36): BBa_K1485002
ggcggtagcggcagcggtagcggtagcggcagcccg

SEQ ID NO: 82 - protein, 12 aa, Artificial Sequence
  Description of Artificial Sequence Synthetic peptide
  MISC_FEATURE (1)..(12): BBa_K1485002
GGSGSGSGSGSP

SEQ ID NO: 83 - DNA, 381 bp, Micavibrio aeruginosavorus
  misc_feature (1)..(381): GN37
atgacatacaccctgagcaaaagaagcctggataacctaaaaggcgttcatcccgatctggttgccgttgtccatcgcgccatccagcttacaccggttgatttcgcggtgatcgaaggcctgcgctccgtatcccgccaaaaggaactggtggccgccggcgccagcaagaccatgaacagccgacacctgacaggccatgcggttgatctagccgcttacgtcaatggcatccgctgggactggcccctgtatgacgccatcgccgtggctgtgaaagccgcagcaaaggaattgggtgtggccatcgtgtggggcggtgactggaccacgtttaaggatggcccgcactttgaactggatcggagcaaatacagatga

SEQ ID NO: 84 - protein, 126 aa, Micavibrio aeruginosavorus
MTYTLSKRSLDNLKGVHPDLVAVVHRAIQLTPVDFAVIEGLRSVSRQKELVAAGASKTMNSRHLTGHAVDLAAYVNGIRWDWPLYDAIAVAVKAAAKELGVAIVWGGDWTTFKDGPHFELDRSKYR

SEQ ID NO: 85 - DNA, 39 bp, Artificial Sequence
  Description of Artificial Sequence Synthetic oligonucleotide
  misc_feature (1)..(39): IGEM linker (BBA_K1486037)
ggcggtggctctggaggtggtgggtccggcggtggctct

SEQ ID NO: 86 - protein, 13 aa, Artificial Sequence
  Description of Artificial Sequence Synthetic peptide
  MISC_FEATURE (1)..(13): IGEM linker (BBA_K1486037)
GGGSGGGGSGGGS

SEQ ID NO: 87 - DNA, 36 bp, Sus scrofa
cgcctgaaaaaaattggcaaagtgctgaaatggatt

SEQ ID NO: 88 - protein, 12 aa, Sus scrofa
RLKKIGKVLKWI

SEQ ID NO: 89 - DNA, 102 bp, Unknown
  Description of Unknown Gokushovirinae sequence
  misc_feature (1)..(102): gkh2
  misc_feature (1)..(102): Description of Unknown Gokushovirinae sequence
atgtcgaagaaggcgtcgaggaagagttttactaagggtgccgttaaggttcataagaaaaatgttcctactcgtgttcctatgcgtggcggtattaggctt

SEQ ID NO: 90 - protein, 34 aa, Unknown
  Description of Unknown Gokushovirinae sequence
MSKKASRKSFTKGAVKVHKKNVPTRVPMRGGIRL

SEQ ID NO: 91 - DNA, 54 bp, Sus scrofa
cgtaaaaaaacccgtaaacgtctgaaaaaaatcggtaaagttctgaaatggatc

SEQ ID NO: 92 - protein, 18 aa, Sus scrofa
RKKTRKRLKKIGKVLKWI

SEQ ID NO: 93 - DNA, 45 bp, Sus scrofa
acccgcaaacgcctgaaaaaaattggcaaagtgctgaaatggatt

SEQ ID NO: 94 - protein, 15 aa, Sus scrofa
TRKRLKKIGKVLKWI

SEQ ID NO: 95 - DNA, 348 bp, Pseudomonas phage PaP2
atgaaactcagcgaaaaacgagcactgttcacccagctgcttgcccagttaattctttgggcaggaactcaggatcgagtgtcagtagccttggatcaagtgaaaaggacacaggctgaagctgatgccaatgctaagtctggagcaggcattaggaactctctccatctactgggattagccggtgatcttatcctctacaaggatggtaaatacatggataagagcgaggattataagttcctgggagattactggaagagtctccatcctctttgtcggtggggcggagattttaaaagccgtcctgatggtaatcatttctccttggaacacgaaggagtgcaa

SEQ ID NO: 96 - protein, 116 aa, Pseudomonas phage PaP2
MKLSEKRALFTQLLAQLILWAGTQDRVSVALDQVKRTQAEADANAKSGAGIRNSLHLLGLAGDLILYKDGKYMDKSEDYKFLGDYWKSLHPLCRWGGDFKSRPDGNHFSLEHEGVQ

SEQ ID NO: 97 - DNA, 30 bp, Artificial Sequence
  Description of Artificial Sequence Synthetic oligonucleotide
  misc_feature (1)..(30): linker
ccaccaaccgcgggcggcaccgcgggcggc

SEQ ID NO: 98 - protein, 10 aa, Artificial Sequence
  Description of Artificial Sequence Synthetic peptide
PPTAGGTAGG

SEQ ID NO: 99 - DNA, 27 bp, Artificial Sequence
  Description of Artificial Sequence Synthetic oligonucleotide
  misc_feature (1)..(27): purification tag GSHHHHHHG
ggatcccatcatcaccaccatcatggt

SEQ ID NO: 100 - protein, 9 aa, Artificial Sequence
  Description of Artificial Sequence Synthetic peptide
GSHHHHHHG

SEQ ID NO: 101 - DNA, 120 bp, Chlamydia phage 4
atggcacgaagatacagactttcgcgacgcagaagtcgacgacttttttcaagaactgcattaagaatgcatcgaagaaatagacttcgaagaattatgcgtggcggcattaggttttag

SEQ ID NO: 102 - protein, 39 aa, Chlamydia phage 4
MARRYRLSRRRSRRLFSRTALRMHRRNRLRRIMRGGIRF

SEQ ID NO: 103 - DNA, 126 bp, Escherichia coli
atggctcgttcccgtagacgtatgtctaagcgttcttcccgccgttcgttccgcaagtatgcgaagtcgcataagaagaactttaaagcccgctcaatgcgtggcggtatccgtttatgataataa

SEQ ID NO: 104 - protein, 39 aa, Escherichia coli
MARSRRRMSKRSSRRSFRKYAKSHKKNFKARSMRGGIRL

SEQ ID NO: 105 - DNA, 114 bp, Chlamydia trachomatis
aaacgtagaaaaatgacaagaaaaggttctaagcgtctttttactgcaactgctgataaaactaaatctatcaatactgccccgccgccaatgcgtggcggtatccggttgtag

SEQ ID NO: 106 - protein, 37 aa, Chlamydia trachomatis
KRRKMTRKGSKRLFTATADKTKSINTAPPPMRGGIRL

SEQ ID NO: 107 - DNA, 114 bp, Oscillibacter sp. PC13
atgagaaagcgaatgtctaagcgtgttgacaagaaggtgttccgtcgtactgccgcatctgccaagaagattaacattgaccccaagatttaccgtggaggtattcgcctatga

SEQ ID NO: 108 - protein, 37 aa, Oscillibacter sp. PC13
MRKRMSKRVDKKVFRRTAASAKKINIDPKIYRGGIRL

SEQ ID NO: 109 - DNA, 36 bp, Artificial Sequence
  Description of Artificial Sequence Synthetic oligonucleotide
  misc_feature (1)..(36): RR12
cgccgcctgattcgcctgtggctgcgcctgctgcgc

SEQ ID NO: 110 - protein, 12 aa, Artificial Sequence
  Description of Artificial Sequence Synthetic peptide
RRLIRLWLRLLR

SEQ ID NO: 111 - DNA, 12 bp, Artificial Sequence
  Description of Artificial Sequence Synthetic oligonucleotide
  misc_feature (1)..(12): structure moiety
atgatcgaccgt

SEQ ID NO: 112 - protein, 4 aa, Artificial Sequence
  Description of Artificial Sequence Synthetic peptide
MIDR

SEQ ID NO: 113 - DNA, 12 bp, Artificial Sequence
  Description of Artificial Sequence Synthetic oligonucleotide
  misc_feature (1)..(12): moiety (outer membrane binding peptide from PMID 22628248)
ttcattcgtctg

SEQ ID NO: 114 - protein, 4 aa, Artificial Sequence
  Description of Artificial Sequence Synthetic peptide
FIRL

SEQ ID NO: 115 - DNA, 12 bp, Artificial Sequence
  Description of Artificial Sequence Synthetic oligonucleotide
  misc_feature (1)..(12): structure moiety
aatccgacccat

SEQ ID NO: 116 - protein, 4 aa, Artificial Sequence
  Description of Artificial Sequence Synthetic peptide
NPTH

SEQ ID NO: 117 - DNA, 477 bp, Artificial Sequence
  Description of Artificial Sequence Synthetic polynucleotide
  misc_feature (1)..(477): GN202 lysin
ggtccgcgtcgtccgcgtcgtccgggtcgtcgtgctccggttcgtacatcccaacgaggcatcgacctcatcaaatccttcgagggcctgcgcctgtccgcttaccaggactcggtgggtgtctggaccataggttacggcaccactcggggcgtcacccgctacatgacgatcaccgtcgagcaggccgagcggatgctgtcgaacgacattcagcgcttcgagccagagctagacaggctggcgaaggtgccactgaaccagaaccagtgggatgccctgatgagcttcgtgtacaacctgggcgcggccaatctggcgtcgtccacgctgctcgacctgctgaacaagggtgactaccagggagcagcggaccagttcccgcattgggtgaatgcgggcggtaagcgcttggatggtctggttaagcgtcgagcagccgagcgtgcgctgttcctggagccactatcgtgataa

SEQ ID NO: 118 - protein, 158 aa, Artificial Sequence
  Description of Artificial Sequence Synthetic polypeptide
MGPRRPRRPGRRAPVRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLDLLNKGDYQGAADQFPHWVNAGGKRLDGLVKRRAAERALFLEPLS

SEQ ID NO: 119 - DNA, 30 bp, Artificial Sequence
  Description of Artificial Sequence Synthetic oligonucleotide
  misc_feature (1)..(30): cationic peptide
aaattctttaagttctttaagttttttaaa

SEQ ID NO: 120 - protein, 10 aa, Artificial Sequence
  Description of Artificial Sequence Synthetic peptide
KFFKFFKFFK

SEQ ID NO: 121 - DNA, 54 bp, Artificial Sequence
  Description of Artificial Sequence Synthetic oligonucleotide
  misc_feature (1)..(54): linker
gccggcgcaggagctggtgcaggagctggtgcaggagctggtgcaggagctagc

SEQ ID NO: 122 - protein, 18 aa, Artificial Sequence
  Description of Artificial Sequence Synthetic peptide
AGAGAGAGAGAGAGAGAS

SEQ ID NO: 123 - DNA, 543 bp, Artificial Sequence
  Description of Artificial Sequence Synthetic polynucleotide
  misc_feature (1)..(543): GN14 lysin
aataacgaacttccttgggtagccgaagcccgaaagtatatcggccttcgcgaagacacttcgaagacttcgcataacccgaaacttcttgccatgcttgaccgcatgggcgaattttccaacgaatcccgcgcttggtggcacgacgacgaaacgccttggtgcggactgttcgtcggctattgcttgggcgttgccgggcgctacgtcgtccgcgaatggtacagggcgcgggcatgggaagccccgcagcttacgaagcttgaccggcccgcatacggcgcgcttgtgaccttcacgcgaagcggcggcggccacgtcggttttattgtgggcaaggatgcgcgcggaaatcttatggttcttggcggtaatcagtcgaacgccgtaagtatcgcaccgttcgcagtatcccgcgtaaccggctatttctggccgtcgttctggcgaaacaagaccgcagttaaaagcgttccgtttgaagaacgttattcgctgccgctgttgaagtcgaacggcgaactttcgacgaatgaagcgtaa

SEQ ID NO: 124 - protein, 180 aa, Artificial Sequence
  Description of Artificial Sequence Synthetic polypeptide
NNELPWVAEARKYIGLREDTSKTSHNPKLLAMLDRMGEFSNESRAWWHDDETPWCGLFVGYCLGVAGRYVVREWYRARAWEAPQLTKLDRPAYGALVTFTRSGGGHVGFIVGKDARGNLMVLGGNQSNAVSIAPFAVSRVTGYFWPSFWRNKTAVKSVPFEERYSLPLLKSNGELSTNEA

SEQ ID NO: 125 - DNA, 471 bp, Artificial Sequence
  Description of Artificial Sequence Synthetic polynucleotide
  misc_feature (1)..(471): GN156
ggtccgcgtcgtccgcgtcgtccgggtcgtcgtgctccggttcgtacctctcagcgtggtatcgacctgatcaaatctttcgaaggtctgcgtctgtctgcttaccaggactctgttggtgtttggaccatcggttacggtaccacccgtggtgttacccgttacatgaccatcaccgttgaacaggctgaacgtatgctgtctaacgacatccagcgtttcgaaccggaactggaccgtctggctaaagttccgctgaaccagaaccagtgggacgctctgatgtctttcgtttacaacctgggtgctgctaacctggcttcttctaccctgctgaaactgctgaacaaaggtgactaccagggtgctgctgaccagttcccgcgttgggttaacgctggtggtaaacgtctggacggtctggttaaacgtcgtgctgctgaacgtgctctgttcctggaaccgctgtct

SEQ ID NO: 126 - protein, 157 aa, Artificial Sequence
  Description of Artificial Sequence Synthetic polypeptide
GPRRPRRPGRRAPVRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLKLLNKGDYQGAADQFPRWVNAGGKRLDGLVKRRAAERALFLEPLS

SEQ ID NO: 127 - protein, 39 aa, Artificial Sequence
  Description of Artificial Sequence Synthetic polypeptide
  MISC_FEATURE (1)..(39): PGN4
NKGDYQGAADQFPRWVNAGGKRLDGLVKRRASQSRESQC

SEQ ID NO: 128 - protein, 42 aa, Artificial Sequence
  Description of Artificial Sequence Synthetic polypeptide
  MISC_FEATURE (1)..(42): FGN4-1
NKGDYQGAADQFPRWVNAGGKRLDGLVKRRAAERALFLEPLS

SEQ ID NO: 129 - protein, 31 aa, Artificial Sequence
  Description of Artificial Sequence Synthetic polypeptide
  MISC_FEATURE (1)..(31): FGN4-2
NKGDYQGAADQFPRWVNAGGKRLDGLVKRRA

SEQ ID NO: 130 - DNA, 54 bp, Artificial Sequence; identical to SEQ ID NO: 91
  Description of Artificial Sequence Synthetic oligonucleotide
  misc_feature (1)..(54): RI18

SEQ ID NO: 131 - protein, 18 aa, Artificial Sequence
  Description of Artificial Sequence Synthetic peptide
RKKTRKRLKKIGKVLKWI

SEQ ID NO: 132 - DNA, 111 bp, Chlamydia virus Chp1
atggttcgtagaagacgtttgagaagaagaataagtagaagaatttttagaagaacagtagctagagttggtagaaggcgaaggtcttttcgtggtggtattagattttaa

SEQ ID NO: 133 - protein, 36 aa, Chlamydia virus Chp1
MVRRRRLRRRISRRIFRRTVARVGRRRRSFRGGIRF

SEQ ID NO: 134 - DNA, 108 bp, Chlamydia virus CPAR39
ttgtgcaaaaaagtgtgcaaaaaatgcccaaaaaaagggccaaaaaatgcccccaaaatcggagcattttacgagagaaaaacacctagacttaaacagtctacttga

SEQ ID NO: 135 - protein, 35 aa, Chlamydia virus CPAR39
MCKKVCKKCPKKGPKNAPKIGAFYERKTPRLKQST

SEQ ID NO: 136 - DNA, 135 bp, Chlamydia phage 3
atgaggttaaaaatggcacgaagaagatacagacttccgcgacgtagaagtcgaagacttttttcaagaactgcattaaggatgcatccaagaaataggcttcgaagaattatgcgtggcggcattaggttctag

SEQ ID NO: 137 - protein, 44 aa, Chlamydia phage 3; identical to SEQ ID NO: 70

SEQ ID NO: 138 - DNA, 117 bp, Chlamydia trachomatis
atgaaacgtagaaaaatgacaagaaaaggttctaagcgtctttttactgcaactgctgataaaactaaatctatcaatactgccccgccgccaatgcgtggcggtatccggttgtaa

SEQ ID NO: 139 - protein, 38 aa, Chlamydia trachomatis
MKRRKMTRKGSKRLFTATADKTKSINTAPPPMRGGIRL

SEQ ID NO: 140 - DNA, 120 bp, Chlamydia trachomatis
atgtctaaaaagcgttctcgcatgtctcgccgccgttctaagaagttgttctcgaaaacggctctccgcacgaagagtgtcaacacccgtccgcctatgcgcggagggttccggttctga

SEQ ID NO: 141 - protein, 39 aa, Chlamydia trachomatis
MSKKRSRMSRRRSKKLFSKTALRTKSVNTRPPMRGGFRF

SEQ ID NO: 142 - DNA, 123 bp, Chlamydia trachomatis
atgtctcttcgtcgtcataagctttctcgtaaggcgtctaagcgtatttttcgtaaaggtgcatcacgcacgaagactttgaatactcgtgctacgcctatgcgcggcggtttccgtatttaa

SEQ ID NO: 143 - protein, 40 aa, Chlamydia trachomatis
MSLRRHKLSRKASKRIFRKGASRTKTLNTRATPMRGGFRI

SEQ ID NO: 144 - DNA, 117 bp, Chlamydia trachomatis
gtgaaacgtcgtaaactgtccaaaaagaaatctcgcaagattttcactcgcggtgctgtaaatgtgaaaaagcgtaaccttcgcgctcgcccaatgcgcggcggtttccggatctaa

SEQ ID NO: 145 - protein, 38 aa, Chlamydia trachomatis
MKRRKLSKKKSRKIFTRGAVNVKKRNLRARPMRGGFRI

SEQ ID NO: 146 - DNA, 114 bp, Chlamydia trachomatis
atggctaaaaaaatgactaaaggcaaggatcgtcaggtttttcgtaaaaccgctgatcgtactaagaaactcaatgttagaccgttgttatatcgaggaggtatcagattatga

SEQ ID NO: 147 - protein, 37 aa, Chlamydia trachomatis
MAKKMTKGKDRQVFRKTADRTKKLNVRPLLYRGGIRL

SEQ ID NO: 148 - DNA, 120 bp, Chlamydia trachomatis
atggcaggaaaaaaaatggtatcaaaaggaaaagatagacagattttccgaaaaactgctgatcgcactaaaaaaatgaatgtgcgcccgctattatatcgtggaggtattagattatga

SEQ ID NO: 149 - protein, 39 aa, Chlamydia trachomatis
MAGKKMVSKGKDRQIFRKTADRTKKMNVRPLLYRGGIRL

SEQ ID NO: 150 - DNA, 126 bp, Marine gokushovirus
atgagaagaccaagaaaaatgaactataaaaaatcaaaaagaatgttttcacgcacagcagcgagaacacacagaaaaaactctctaagaggtagccgacctatgagaggcggaatacgtctttaa

SEQ ID NO: 151 - protein, 41 aa, Marine gokushovirus
MRRPRKMNYKKSKRMFSRTAARTHRKNSLRGSRPMRGGIRL

SEQ ID NO: 152 - DNA, 108 bp, Unknown
  Description of Unknown Bacteria; environmental sample sequence
atgaaaatgcgtaagcggacggacaagcgagtgtttacccgcaccgctgctaagtccaagaaagtgaacattgccccgaaaatttttagaggaggtatccgtctgtga

SEQ ID NO: 153 - protein, 35 aa, Unknown
  Description of Unknown Bacteria; environmental sample sequence
MKMRKRTDKRVFTRTAAKSKKVNIAPKIFRGGIRL

SEQ ID NO: 154 - DNA, 120 bp, Escherichia sp.
atggctcgttctcgccgtcgtatgtccaagcgttcttcccgtcgttcgttccgtaagtacgcaaagacgcataaacgtaactttaaagcccgctctatgcgtggtggaattcgtctttga

SEQ ID NO: 155 - protein, 39 aa, Escherichia sp.
MARSRRRMSKRSSRRSFRKYAKTHKRNFKARSMRGGIRL

SEQ ID NO: 156 - DNA, 144 bp, Cognatishimia maritima
atggaaagcccgaacagccgcagccagctgggcattaccctgtatctgctgagcaccatttttccggatgcgtgctttcgctatcgccgcgaactgccgtatccgctggtgatttggggcgtggcgaccctgtgcctgcagtaa

SEQ ID NO: 157 - protein, 47 aa, Cognatishimia maritima
MESPNSRSQLGITLYLLSTIFPDACFRYRRELPYPLVIWGVATLCLQ

SEQ ID NO: 158 - DNA, 114 bp, Unknown
  Description of Unknown Bacteria; environmental sample sequence
atgagacgtcgtcgtctatcccgcagaacttcccgccgttttttccgtaaaggacttaaggttcgccgtcgtaacctccgcgcgagacccatgagaggcggattcagaatttga

SEQ ID NO: 159 - protein, 37 aa, Unknown
  Description of Unknown Bacteria; environmental sample sequence
MRRRRLSRRTSRRFFRKGLKVRRRNLRARPMRGGFRI

SEQ ID NO: 160 - DNA, 120 bp, Unknown
  Description of Unknown Bacteria; environmental sample sequence
atggcacgacgcaagaagatgaaaggcaagcgggataaacgggtgtttaagcagacagccaacaaaaccaaggctatcaacatcagcccaaaaaacatgagagggggtacgagactgtga

SEQ ID NO: 161 - protein, 39 aa, Unknown
  Description of Unknown Bacteria; environmental sample sequence
MARRKKMKGKRDKRVFKQTANKTKAINISPKNMRGGTRL

SEQ ID NO: 162 - DNA, 162 bp, Marine gokushovirus
atgttaactgtgtggagtgacacccctaccataaaaaggagaaaagacatgtatagaaagagaatgtcaagaaagaaaagtaaaaaggtttttgcaaaaaccgcaatgaaagtaaataaaagaaaccacgttaaacctatgcgtggtggatatagaatataa

SEQ ID NO: 163 - protein, 53 aa, Marine gokushovirus
MLTVWSDTPTIKRRKDMYRKRMSRKKSKKVFAKTAMKVNKRNHVKPMRGGYRI

SEQ ID NO: 164 - DNA, 120 bp, Marine gokushovirus
atgatgaagtacagaaaaaaaatgagcgctaaaagtagccgaaagcaatttacaaaaggcgccatgaaagtgaagggtaaaaacttcacaaaaccaatgcgcggaggcatccgtctatag

SEQ ID NO: 165 - protein, 39 aa, Marine gokushovirus
MMKYRKKMSAKSSRKQFTKGAMKVKGKNFTKPMRGGIRL

SEQ ID NO: 166 - DNA, 117 bp, Marine gokushovirus
atgcgacgttacaatgtaaataaaggtaaatctgctaagaagtttcgaaagcaggtaagtaagacgaaggttgcaaacctacgttctaatccaatgcgaggtggttggagactctaa

SEQ ID NO: 167 - protein, 38 aa, Marine gokushovirus
MRRYNVNKGKSAKKFRKQVSKTKVANLRSNPMRGGWRL

SEQ ID NO: 168 - DNA, 87 bp, Spiroplasma virus SpV4
atggcttatcgtggttttaaaacgagtcgtgttgtaaaacatagagtacgtagaagatggtttaatcatagaagacgttatagatag

SEQ ID NO: 169 - protein, 28 aa, Spiroplasma virus SpV4
MAYRGFKTSRVVKHRVRRRWFNHRRRYR

SEQ ID NO: 170 - DNA, 117 bp, Spiroplasma virus SpV4
gtgagacgcaaggttaagaacacaaagcgtcatcagtggaggttgactcattctgcacgttcaattaaacgtgctaatataatgccgtcaaatcctcgtggtggacgtcgtttttag

SEQ ID NO: 171 - protein, 38 aa, Spiroplasma virus SpV4
MRRKVKNTKRHQWRLTHSARSIKRANIMPSNPRGGRRF

SEQ ID NO: 172 - DNA, 798 bp, Pseudomonas phage PhiPA3
atgacattactgaagaaaggcgacaagggtgacgccgtaaaacaactacagcagaaactcaaagaccttgggtataccctgggtgtcgatggcaacttcggtaatggcaccgatactgtcgttcgttctttccaaaccaaaatgaagcttagtgttgatggtgtggttggtaatggtactatgagtactattgactctactctagcaggcattaaagcgtggaagactagtgtacctttccctgcgacgaacaaatcccgagcaatggcaatgccaacgttgactgaaataggtcgactgacaaacgttgatcctaaattgctagcgacattctgttctatcgaaagcgcgtttgattacacagctaaaccctacaagcccgatggcacagtgtacagctccgccgaaggttggttccagttcctggatgcaacatgggatgacgaagtgcgtaaacacggtaagcaatatagcttccctgttgatcctggtcgttctttgcgtaaagatccacgggctaatggcttgatgggcgctgagttcctcaaagggaatgctgctattctgcggccagtactgggtcatgaaccgagcgacacagatctttatctagcccatttcatgggagcaggtggcgcaaaacagttccttatggccgatcaaaataaattggctgccgaattgttccctggtccagctaaggctaatcctaacatcttctataaatccggaaatattgcccgcactttagcagaggtctatgcagtcctcgatgctaaggtagccaagcatagagct

SEQ ID NO: 173 - protein, 266 aa, Pseudomonas phage PhiPA3
MTLLKKGDKGDAVKQLQQKLKDLGYTLGVDGNFGNGTDTVVRSFQTKMKLSVDGVVGNGTMSTIDSTLAGIKAWKTSVPFPATNKSRAMAMPTLTEIGRLTNVDPKLLATFCSIESAFDYTAKPYKPDGTVYSSAEGWFQFLDATWDDEVRKHGKQYSFPVDPGRSLRKDPRANGLMGAEFLKGNAAILRPVLGHEPSDTDLYLAHFMGAGGAKQFLMADQNKLAAELFPGPAKANPNIFYKSGNIARTLAEVYAVLDAKVAKHRA

SEQ ID NO: 174 - DNA, 435 bp, Artificial Sequence
  Description of Artificial Sequence Synthetic polynucleotide
  misc_feature (1)..(435): GN37 and RI18
atgacatacaccctgagcaaaagaagcctggataacctaaaaggcgttcatcccgatctggttgccgttgtccatcgcgccatccagcttacaccggttgatttcgcggtgatcgaaggcctgcgctccgtatcccgccaaaaggaactggtggccgccggcgccagcaagaccatgaacagccgacacctgacaggccatgcggttgatctagccgcttacgtcaatggcatccgctgggactggcccctgtatgacgccatcgccgtggctgtgaaagccgcagcaaaggaattgggtgtggccatcgtgtggggcggtgactggaccacgtttaaggatggcccgcactttgaactggatcggagcaaatacagatgacgtaaaaaaacccgtaaacgtctgaaaaaaatcggtaaagttctgaaatggatc

SEQ ID NO: 175 - protein, 144 aa, Artificial Sequence
  Description of Artificial Sequence Synthetic polypeptide
MTYTLSKRSLDNLKGVHPDLVAVVHRAIQLTPVDFAVIEGLRSVSRQKELVAAGASKTMNSRHLTGHAVDLAAYVNGIRWDWPLYDAIAVAVKAAAKELGVAIVWGGDWTTFKDGPHFELDRSKYRRKKTRKRLKKIGKVLKWI

SEQ ID NO: 176 - DNA, 120 bp, Escherichia sp.
atggctcgttctcgtcgtcgtatgtctaaacgttcttctcgtcgttcttttcgtaaatatgctaaaactcataaaaaaaattttaaagctcgttctatgcgtggaggaattcgtttataa

SEQ ID NO: 177 - protein, 39 aa, Escherichia sp.
MARSRRRMSKRSSRRSFRKYAKTHKKNFKARSMRGGIRL

SEQ ID NO: 178 - DNA, 117 bp, Escherichia coli
atggcgcgcagccgccgccgcatgagcaaacgcagcagccgccgcagctttcgcaaatatgcgaaaagccataaaaaaaactttaaagcgcgcagcatgcgcggcggcattcgcctg

SEQ ID NO: 179 - protein, 39 aa, Escherichia coli; identical to SEQ ID NO: 104

SEQ ID NO: 180 - DNA, 117 bp, Alces alces faeces associated microvirus MP12 5423
atggcaaagaaaattagaaacaaagcacgtgatagacgtatcttcacaagaacagcttcacgcatgcacaaggcaaaccgcacaccaagatttatgagaggcggtattaggttatga

SEQ ID NO: 181 - protein, 38 aa, Alces alces faeces associated microvirus MP12 5423
MAKKIRNKARDRRIFTRTASRMHKANRTPRFMRGGIRL

SEQ ID NO: 182 - DNA, 117 bp, Unknown
  Description of Unknown Gokushovirinae environmental sample sequence
atgcgtcgtaaaaaaatgtcacgcggtaaatcaaaaaaactctttcgccgaacagcaaaacgcgttcatcgaaaaaacctacgagctcgcccaatgcgtggcggcatacgcatgtag

SEQ ID NO: 183 - protein, 38 aa, Unknown
  Description of Unknown Gokushovirinae environmental sample sequence
MRRKKMSRGKSKKLFRRTAKRVHRKNLRARPMRGGIRM

SEQ ID NO: 184 - DNA, 120 bp, Unknown
  Description of Unknown Gokushovirinae environmental sample sequence
atggcgaagcgacacaaaatcccgcaacgcgcgtcacaacattccttcacgcgccatgcgcaaaaggtccaccctaagaacgttccccgcctgccaatgcgaggcggtatccgtctctaa

SEQ ID NO: 185 - protein, 39 aa, Unknown
  Description of Unknown Gokushovirinae environmental sample sequence
MAKRHKIPQRASQHSFTRHAQKVHPKNVPRLPMRGGIRL

SEQ ID NO: 186 - DNA, 114 bp, Unknown
  Description of Unknown uncultured bacterium sequence
atgcgtaaaaaaatgcacaaatcattagacaagcgagtgtttaaccgcactgcaaaaaaatcaaaaaaaataaatgttaatcctgtagtttatcgtggaggtattagattatga

SEQ ID NO: 187 - protein, 37 aa, Unknown
  Description of Unknown uncultured bacterium sequence
MRKKMHKSLDKRVFNRTAKKSKKINVNPVVYRGGIRL

SEQ ID NO: 188 - DNA, 117 bp, Marine gokushovirus; identical to SEQ ID NO: 166

SEQ ID NO: 189 - protein, 38 aa, Marine gokushovirus; identical to SEQ ID NO: 167

SEQ ID NO: 190 - DNA, 126 bp, Richelia intracellularis HH01
atgcgtccagttaaaagatcaagagtaaataaggcccgatctgcaggcaagtttcgtaagcaggtcggtaaaacaaagatggcaaatctgcgtagtaatccgatgcgcggcggatggcggctgtga

SEQ ID NO: 191 - protein, 41 aa, Richelia intracellularis HH01
MRPVKRSRVNKARSAGKFRKQVGKTKMANLRSNPMRGGWRL

SEQ ID NO: 192 - DNA, 126 bp, Gokushovirinae Fen7875_21
atgaagccattgaagcgtaagccggttcagaaggcgcggtcagcagccaagttccgtcgaaatgtgtctaccgttaaggctgccaatatggcggtgaagccgatgcgcggcggttggcggttctga

SEQ ID NO: 193 - protein, 41 aa, Gokushovirinae Fen7875_21
MKPLKRKPVQKARSAAKFRRNVSTVKAANMAVKPMRGGWRF

SEQ ID NO: 194 - DNA, 135 bp, Mycobacterium phage BabyRay
atgaccaagagagacatcgagtaccggaaagctttggggctcaacccatctgagccgctcccgaagattgtgggtgccgtcacccgccacggggccactctgaaacgcccacgggtcaccgcactggcccgatag

SEQ ID NO: 195 - protein, 44 aa, Mycobacterium phage BabyRay
MTKRDIEYRKALGLNPSEPLPKIVGAVTRHGATLKRPRVTALAR

SEQ ID NO: 196 - DNA, 117 bp, Bdellovibrio phage phiMH2K
atgaaaagaaaaccaatgagccgcaaggcctctcaaaaaaccttcaaaaagaacacaggcgttcaacgcatgaaccatctcaacccacgcgccatgcgtggtggcattagactataa

SEQ ID NO: 197 - protein, 38 aa, Bdellovibrio phage phiMH2K
MKRKPMSRKASQKTFKKNTGVQRMNHLNPRAMRGGIRL

SEQ ID NO: 198 - DNA, 168 bp, Pseudomonas phage PP7
ttgtcgtcaaccttgtgccgctgggccgttaaggccctgcggtgtacccgtgtgtataaggagtttatatggaaacccttagtagcgctcagttacgtgacgttgtatcttctgagctcggtcttcctgtcccaactcagctaccccatcgggagctgggcggtgtag

SEQ ID NO: 199 - protein, 55 aa, Pseudomonas phage PP7
MSSTLCRWAVKALRCTRVYKEFIWKPLVALSYVTLYLLSSVFLSQLSYPIGSWAV

SEQ ID NO: 200 - DNA, 108 bp, Acinetobacter phage AP205
atgaagaaaaggacaaaagccttgcttccctatgcggttttcatcatactcagctttcaactaacattgttgactgccttgtttatgtattaccattataccttttag

SEQ ID NO: 201 - protein, 35 aa, Acinetobacter phage AP205
MKKRTKALLPYAVFIILSFQLTLLTALFMYYHYTF

SEQ ID NO: 202 - DNA, 558 bp, Acinetobacter phage vB_AbaP_CEB1
atgattctgactaaagatgggtttggtattatccgtaatgaactattcggaggtaagttagatcaaactcaagtagatgcaataaactttattgtagagaaagctactgagtctggtttatcttatccagaggcagcctatttactagctaccatctatcatgagactggtctaccaagcggttatcgaactatgcaacctattaaagaagctggttctgataactaccttcgatctaagaagtactacccgtacattggttatggttatgtacagttaacttggaaggagaactatggacggattggtaaacttattggaattgacctaattaagaatcctgagaaagcgctagaacctttaattgctattcagattgctatcaaaggcatgttgaatggttggttcacaggtgttggattccgacgtaaacgtccagttagtaaatacaacaaacagcagtacatagctgcgcgtaatatcattaatgggaaagataaggctgagcttatagcgaagtacgctattatctttgaacgcgctctacggagcttataa

SEQ ID NO: 203 - protein, 185 aa, Acinetobacter phage vB_AbaP_CEB1
MILTKDGFGIIRNELFGGKLDQTQVDAINFIVEKATESGLSYPEAAYLLATIYHETGLPSGYRTMQPIKEAGSDNYLRSKKYYPYIGYGYVQLTWKENYGRIGKLIGIDLIKNPEKALEPLIAIQIAIKGMLNGWFTGVGFRRKRPVSKYNKQQYIAARNIINGKDKAELIAKYAIIFERALRSL

SEQ ID NO: 204 - protein, 36 aa, Sus scrofa
  MISC_FEATURE (1)..(36): PMAP-36
GRFRRLRKKTRKRLKKIGKVLKWIPPIVGSIPLGCG

(19) United States
(12) Patent Application Publication
(10) Pub. No.: US 2021/0363511 A1
(43) Pub. Date: Nov. 25, 2021

(54) LYSIN-ANTIMICROBIAL PEPTIDE (AMP) POLYPEPTIDE CONSTRUCTS, LYSINS, ISOLATED POLYNUCLEOTIDES ENCODING SAME AND USES THEREOF

(71) Applicant: CONTRAFECT CORPORATION, Yonkers, NY (US)

(72) Inventor: Raymond SCHUCH, Mountain Lakes, NJ (US)

(21) Appl. No.: 17/041,853

(22) PCT Filed: Aug. 23, 2019
(86) PCT No.: PCT/US19/47916

§ 371 Date: Sep. 25, 2020

Related U.S. Application Data
(63) Continuation of application No. PCT/US2019/024912, filed on Mar. 29, 2019.
(60) Provisional application No. 62/721,969, filed on Aug. 23, 2018.
(60) Provisional application No. 62/722,793, filed on Aug. 24, 2018.
(60) Provisional application No. 62/849,320, filed on May 17, 2019.
(60) Provisional application No. 62/860,836, filed on Jun. 13, 2019.

(51) Int. Cl. C12N 9/36; C07K 14/47; C07K 7/08; C07K 7/06; A61K 31/407

(52) U.S. Cl. CPC C12N 9/2462; C07K 14/4703; A61K 38/00; C07K 7/06; A61K 31/407; C07K 7/08

(57) ABSTRACT

The present disclosure is directed to lysin-AMP polypeptide constructs, isolated lysin polypeptides, and pharmaceutical compositions comprising the isolated polypeptides and/or lysin-AMP polypeptide constructs. Methods of using the lysin-AMP polypeptide constructs, isolated lysin polypeptides and pharmaceutical compositions are also herein provided, including methods of treating a bacterial infection of an organ or tissue in which pulmonary surfactant is present or Gram-negative bacterial infections that are associated with a biofilm. In addition, isolated polynucleotides encoding the lysin-AMP polypeptide constructs and isolated lysin polypeptides are disclosed herein.

### CROSS-REFERENCE TO RELATED APPLICATIONS

This application claims the benefit of, and relies on the filing date of PCT Application No. PCT/US2019/024912, filed on 29 Mar. 2019, which claims the benefit of priority of U.S. provisional Application No. 62/722,793, filed 24 Aug. 2018, U.S. Provisional Application No. 62/650,235, filed on 29 Mar. 2018, and U.S. Provisional Application No. 62/721,969, filed on 23 Aug. 2018, and also relies on the filing date of U.S. Provisional Application No. 62/849,320 filed on 17 May 2019 and U.S. Provisional Application No. 62/860,836 filed 13 Jun. 2019, each of which is herein incorporated by reference in its entirety.

### SEQUENCE LISTING

The instant application contains a Sequence Listing which has been submitted electronically in ASCII format and is hereby incorporated by reference in its entirety. Said ASCII copy, created on Aug. 22, 2019, is named 0341_0021-00-304_ST25.txt and is 249,856 bytes in size.

### FIELD OF THE DISCLOSURE

The present disclosure relates to the field of antibacterial agents and more specifically to polypeptides having lysin activity against Gram-negative bacteria and the use of these agents in killing Gram-negative bacteria and combating bacterial infection and contamination.

### BACKGROUND

Gram-negative bacteria, in particular, members of the genus Pseudomonas and the emerging multi-drug resistant pathogen Acinetobacter baumannii, are an important cause of serious and potentially life-threatening invasive infections. Pseudomonas infection presents a major problem in burn wounds, chronic wounds, chronic obstructive pulmonary disorder (COPD), cystic fibrosis, surface growth on implanted biomaterials, and within hospital surface and water supplies where it poses a host of threats to vulnerable patients.

Once established in a patient, P. aeruginosa can be especially difficult to treat. The genome encodes a host of resistance genes, including multidrug efflux pumps and enzymes conferring resistance to beta-lactam and aminoglycoside antibiotics, making therapy against this Gram-negative pathogen particularly challenging due to the lack of novel antimicrobial therapeutics. This challenge is compounded by the ability of P. aeruginosa to grow in a biofilm, which may enhance its ability to cause infections by protecting bacteria from host defenses and chemotherapy.

In the healthcare setting, the incidence of drug-resistant strains of Pseudomonas aeruginosa is increasing. In an observational study of health care-associated bloodstream infections (BSIs) in community hospitals, P. aeruginosa was one of the top four Multiple Drug Resistant (MDR) pathogens, contributing to an overall hospital mortality of 18%. Additionally, outbreaks of MDR P. aeruginosa are well-documented. Poor outcomes are associated with MDR stains of P. aeruginosa that frequently require treatment with drugs of last resort, such as colistin.

Moreover, reduced effectiveness of certain antibiotics is observed in combating infections due to factors in the environment of the infection, such as the pulmonary surfactant, rather than to antibiotic resistance developments. Certain antibiotics, such as daptomycin, for example, have failed to meet criteria in a clinical trial for severe community-acquired pneumonia. This deficiency has been shown to be due to an interaction between daptomycin and pulmonary surfactant, which inhibits the activity of this antibiotic, specifically in the lung environment and more generally in the airway environment wherein pulmonary surfactant is present. Silverman, J. A. et al., “Surfactant Inhibition of Daptomycin,” JID, 191: 2149-2152 (2005). Thus, daptomycin is not indicated for treatment of lung and more generally airway (especially lower respiratory tract) infections and those of skill in the art would not employ a treatment regimen including daptomycin to treat such infections. The inability of daptomycin to combat infection in the presence of pulmonary surfactants has been shown dramatically in, for example, Koplowicz, Y. B. et al., “Development of daptomycin-susceptible methicillin-resistant Staphylococcus aureus Pneumonia during high-dose daptomycin therapy”, Clin Infect Dis. 49(8):1286-7 (2009). Recent studies have focused on overcoming daptomycin inactivity in the presence of surfactant by testing and evaluating antibacterial activity of hybrid molecules of the structurally related lipopeptide A54145. Nguyen, K. T. et al., “Genetically engineered lipopeptide antibiotics related to A54145 and daptomycin with improved properties”, Antimicrob. Agents Chemother. 2010 April; 54(4):1404-1413.

Pulmonary surfactant, a primary component of epithelial lining fluid, is a complex lipid-and-protein mixture that coats the interior surface of the airway, reducing surface tension within the alveoli. Surfactant is composed primarily of dipalmitoylphosphatidylcholine (˜80% in all mammalian species), along with significant amounts of phosphatidylglycerol (PG) and smaller amounts of minor phospholipids, neutral lipids, and cholesterol. There are 4 protein components: hydrophilic proteins SP-A and SP-D and hydrophobic proteins SP-B and SP-C. Goerke, J., “Pulmonary Surfactant: functions and molecular composition”, Biochim. Biophys. Acta. 1998; 1408:79-89. Daptomycin is inserted into artificial membrane vesicles composed of phosphatidylcholine (PC) and PC/PG. Lakey J. H. et al., “Fluorescence indicates a calcium-dependent interaction between the lipopeptide antibiotic LY146032 and phospholipid membranes,” Biochemistry 1988; 27:4639-45; Jung, D. et al., “Structural transitions as determinants of the action of the calcium-dependent antibiotic daptomycin”, Chem. Biol. 2004; 11:949-57.

Thus, to the extent that otherwise effective antibiotics are inhibited by factors present in the organ or tissue that is the site of the infection, such as pulmonary surfactant in the case of infections of the lungs or other airways and more generally of the respiratory tract, a treatment regimen that would restore and even augment activity of such antibiotics would be of commercial and public health value.

In addition to daptomycin discussed above, other antibiotics that are known to be inhibited by pulmonary surfactant include without limitation: tobramycin, an aminoglycoside used to treat infections caused by the gram-negative bacterium Pseudomonas aeruginosa, a common cause of pneumonia (van't Veen, A. et al., “Influence of pulmonary surfactant on in vitro bactericidal activities of amoxicillin, ceftazidime, and tobramycin”, Antimicrob. Agents Chemother. 39:329-333 (1995)), and colistin, a cyclic lipopeptide (polymyxin) broadly active against gram-negative bacteria, including P. aeruginosa. Schwameis, R. et al., “Effect of Pulmonary surfactant on antimicrobial activity in vitro”, Antimicrob. Agents Chemother. 57(10):5151-54 (2013).

To address the need for new antimicrobials with novel mechanisms, researchers are investigating a variety of drugs and biologics. One such class of antimicrobial agents includes lysins. Lysins are cell wall peptidoglycan hydrolases, which act as “molecular scissors” to degrade the peptidoglycan meshwork responsible for maintaining cell shape and for withstanding internal osmotic pressure. Degradation of peptidoglycan results in osmotic lysis. However, lysins, typically, have not been effective against Gram-negative bacteria, at least in part, due to the presence of an outer membrane (OM), which is absent in Gram-positive bacteria and which limits access to subjacent peptidoglycan. Modified lysins (“artilysins”) have also been developed. These agents, which contain lysins fused to specific α-helical domains with polycationic, amphipathic, and hydrophobic features, are capable of translocating across the OM. However, artilysins typically exhibit low in vivo activity.

Although recent publications have described novel lysins that may be used against Gram-negative bacteria with varying levels of efficacy in vivo, there remains a continuing medical need for additional antibacterials that retain activity in human blood matrices or pulmonary surfactant to target MDR P. aeruginosa and other Gram-negative bacteria for the treatment of invasive infections.

### SUMMARY

The present application is directed to novel polypeptide constructs comprising lysins and antimicrobial peptides (AMP) that can be used, for example, to treat bacterial infections, including infections caused by Gram-negative bacteria, particularly multi-drug resistant Gram-negative bacteria, including, but not limited to Pseudomonas aeruginosa. Newly identified lysins and variants thereof, as well as variants of other lysins are also provided. As described herein, the lysin-AMP polypeptide constructs, newly obtained lysins and variant lysins may be included in pharmaceutical compositions that can be used, for example, to treat bacterial infections. Also provided herein, inter alia, are methods for using the lysin-AMP polypeptide constructs, newly identified lysins and variant lysins for treating bacterial infections, augmenting the efficacy of antibiotics and, generally, inhibiting the growth, reducing the population, or killing Gram-negative bacteria, such as P. aeruginosa. Lysin variant polypeptides and polynucleotides encoding the constructs and lysin variants are also provided. In certain embodiments, the lysin-AMP polypeptide constructs, newly obtained lysins and variant lysins may be used to treat bacterial infections in an organ or tissue in which pulmonary surfactant is present, such as, for example, pneumonia (including hospital acquired pneumonia) and cystic fibrosis. In other embodiments, the lysin-AMP polypeptide constructs, newly obtained lysins and variant lysins may be used to treat Gram-negative bacterial infections that are associated with biofilms.

In one aspect, the present disclosure is directed to a lysin-AMP polypeptide construct comprising: (a) a first component comprising the polypeptide sequence of: (i) a lysin selected from the group consisting of GN76 (SEQ ID NO: 203), GN4 (SEQ ID NO: 74), GN146 (SEQ ID NO: 78), GN14 (SEQ ID NO: 124), GN37 (SEQ ID NO: 84) optionally with a single pI-increasing mutation, GN316 (SEQ ID NO: 22) optionally with a single point mutation, lysin Pap2_gp17 (SEQ ID NO: 96), GN329 (SEQ ID NO: 26), GN424 (SEQ ID NO: 56), GN202 (SEQ ID NO: 118), GN425 (SEQ ID NO: 58), GN428 (SEQ ID NO: 60), GN431 (SEQ ID NO: 64), GN486 (SEQ ID NO: 66), GN333 (SEQ ID NO: 28), GN485 (SEQ ID NO: 68), GN123 (SEQ ID NO: 173) and GN121 (SEQ ID NO: 175); or (ii) a polypeptide having lysin activity and having at least 80% sequence identity with the polypeptide sequence of at least one of SEQ ID NOS: 203, 74, 78, 124, 84, 22, 96, 26, 56, 118, 58, 60, 64, 66, 28, 68, 173 or 175; or (iii) an active fragment of the lysin; and

(b) a second component comprising the polypeptide sequence of: (i) at least one antimicrobial peptide (AMP) selected from the group consisting of Chp1 (SEQ ID NO: 133), Chp2 (SEQ ID NO: 70), CPAR39 (SEQ ID NO: 135), Chp3 (SEQ ID NO: 137), Chp4 (SEQ ID NO: 102), Chp6 (SEQ ID NO: 106), Chp7 (SEQ ID NO: 139), Chp8 (SEQ ID NO: 141), Chp9 (SEQ ID NO: 143), Chp10 (SEQ ID NO: 145), Chp11 (SEQ ID NO: 147), Chp12 (SEQ ID NO: 149), Gkh1 (SEQ ID NO: 151), Gkh2 (SEQ ID NO: 90), Unp1 (SEQ ID NO: 153), Ecp1 (SEQ ID NO: 155), Ecp2 (SEQ ID NO: 104), Tma1 (SEQ ID NO: 157), Osp1 (SEQ ID NO: 108), Unp2 (SEQ ID NO: 159), Unp3 (SEQ ID NO: 161), Gkh3 (SEQ ID NO: 163), Unp5 (SEQ ID NO: 165), Unp6 (SEQ ID NO: 167), Spi1 (SEQ ID NO: 169), Spi2 (SEQ ID NO: 171), Ecp3 (SEQ ID NO: 177), Ecp4 (SEQ ID NO: 179), ALCES1 (SEQ ID NO: 181), AVQ206 (SEQ ID NO: 183), AVQ244 (SEQ ID NO: 185), CDL907 (SEQ ID NO: 187), AGT915 (SEQ ID NO: 189), HH3930 (SEQ ID NO: 191), Fen7875 (SEQ ID NO: 193), SBR77 (SEQ ID NO: 195), Bdp1 (SEQ ID NO: 197), LVP1 (SEQ ID NO: 199), Lvp2 (SEQ ID NO: 201), an esculentin fragment (SEQ ID NO: 80), RI12 (SEQ ID NO: 88), TI15 (SEQ ID NO: 94), RI18 (SEQ ID NO: 92), FIRL (SEQ ID NO: 114), a fragment of LPS binding protein (SEQ ID NO: 76), RR12whydro (SEQ ID NO: 110), RI18 peptide derivative (SEQ ID NO: 131) and cationic peptide (SEQ ID NO: 120) or (ii) a polypeptide having AMP activity, wherein the polypeptide is at least 80% identical to at least one of SEQ ID NOS: 133, 70, 135, 137, 102, 106, 139, 141, 143, 145, 147, 149, 151, 90, 153, 155, 104, 157, 108, 159, 161, 163, 165, 167, 169, 171, 177, 179, 181, 183, 185, 187, 189, 191, 193, 195, 197, 199, 201, 80, 88, 94, 92, 114, 76, 110, 131 and 120, wherein the lysin-AMP polypeptide construct comprises at least one activity selected from inhibiting P. aeruginosa bacterial growth, reducing a P. aeruginosa bacterial population and/or killing P. aeruginosa in the absence and/or presence of human serum or in the presence of pulmonary surfactant.

In another aspect, the present disclosure is directed to an isolated polypeptide comprising a lysin selected from the group consisting of GN121 (SEQ ID NO: 175), GN217 lysin (SEQ ID NO: 8), GN394 lysin (SEQ ID NO: 48), GN396 lysin (SEQ ID NO: 50), GN408 lysin (SEQ ID NO: 52), GN418 lysin (SEQ ID NO: 54), GN428 (SEQ ID NO: 60), and GN486 (SEQ ID NO: 66) or an active fragment thereof, wherein the lysin or active fragment thereof inhibits P. aeruginosa bacterial growth, reduces a P. aeruginosa bacterial population and/or kills P. aeruginosa in the absence and/or presence of human serum or in the presence of pulmonary surfactant.

The present disclosure is also directed to an isolated polynucleotide comprising a nucleic acid molecule encoding a lysin-antimicrobial peptide (AMP) polypeptide construct, the nucleic acid molecule comprising:

(a) a first nucleic acid molecule encoding a first component comprising: (i) a lysin selected from the group consisting of GN76 (SEQ ID NO: 203), GN4 (SEQ ID NO: 74), GN146 (SEQ ID NO: 78), GN14 (SEQ ID NO: 124), GN37 (SEQ ID NO: 84) optionally with a single pI-increasing mutation, GN316 (SEQ ID NO: 22) optionally with a single point mutation, lysin Pap2_gp17 (SEQ ID NO: 96), GN329 (SEQ ID NO: 26), GN424 (SEQ ID NO: 56), GN202 (SEQ ID NO: 118), GN425 (SEQ ID NO: 58), GN428 (SEQ ID NO: 60), GN431 (SEQ ID NO: 64), GN486 (SEQ ID NO: 66), GN333 (SEQ ID NO: 28), GN485 (SEQ ID NO: 68), GN123 (SEQ ID NO: 173) and GN121 (SEQ ID NO: 175); or (ii) a polypeptide having lysin activity, wherein the polypeptide is at least 80% identical to at least one of SEQ ID NOS: 203, 74, 78, 124, 84, 22, 96, 26, 56, 118, 58, 60, 64, 66, 28, 68, 173 or 175; or (iii) an active fragment of the lysin; and
(b) a second nucleic acid molecule encoding a second component comprising: (i) at least one antimicrobial peptide (AMP) selected from the group consisting of Chp1 (SEQ ID NO: 133), Chp2 (SEQ ID NO: 70), CPAR39 (SEQ ID NO: 135), Chp3 (SEQ ID NO: 137), Chp4 (SEQ ID NO: 102), Chp6 (SEQ ID NO: 106), Chp7 (SEQ ID NO: 139), Chp8 (SEQ ID NO: 141), Chp9 (SEQ ID NO: 143), Chp10 (SEQ ID NO: 145), Chp11 (SEQ ID NO: 147), Chp12 (SEQ ID NO: 149), Gkh1 (SEQ ID NO: 151), Gkh2 (SEQ ID NO: 90), Unp1 (SEQ ID NO: 153), Ecp1 (SEQ ID NO: 155), Ecp2 (SEQ ID NO: 104), Tma1 (SEQ ID NO: 157), Osp1 (SEQ ID NO: 108), Unp2 (SEQ ID NO: 159), Unp3 (SEQ ID NO: 161), Gkh3 (SEQ ID NO: 163), Unp5 (SEQ ID NO: 165), Unp6 (SEQ ID NO: 167), Spi1 (SEQ ID NO: 169), Spi2 (SEQ ID NO: 171), Ecp3 (SEQ ID NO: 177), Ecp4 (SEQ ID NO: 179), ALCES1 (SEQ ID NO: 181), AVQ206 (SEQ ID NO: 183), AVQ244 (SEQ ID NO: 185), CDL907 (SEQ ID NO: 187), AGT915 (SEQ ID NO: 189), HH3930 (SEQ ID NO: 191), Fen7875 (SEQ ID NO: 193), SBR77 (SEQ ID NO: 195), Bdp1 (SEQ ID NO: 197), LVP1 (SEQ ID NO: 199), Lvp2 (SEQ ID NO: 201), an esculentin fragment (SEQ ID NO: 80), RI12 (SEQ ID NO: 88), TI15 (SEQ ID NO: 94), RI18 (SEQ ID NO: 92), FIRL (SEQ ID NO: 114), a fragment of LPS binding protein (SEQ ID NO: 76), RR12whydro (SEQ ID NO: 110), RI18 peptide derivative (SEQ ID NO: 131) and cationic peptide (SEQ ID NO: 120) or (ii) a polypeptide having AMP activity, wherein the polypeptide is at least 80% identical to at least one of SEQ ID NOS: 133, 70, 135, 137, 102, 106, 139, 141, 143, 145, 147, 149, 151, 90, 153, 155, 104, 157, 108, 159, 161, 163, 165, 167, 169, 171, 177, 179, 181, 183, 185, 187, 189, 191, 193, 195, 197, 199, 201, 80, 88, 94, 92, 114, 76, 110, 131 and 120, wherein the lysin-AMP polypeptide construct comprises at least one activity selected from inhibiting P. aeruginosa bacterial growth, reducing a P. aeruginosa bacterial population and/or killing P. aeruginosa in the absence and/or presence of human serum or in the presence of pulmonary surfactant.

In yet another aspect, the present disclosure is directed to an isolated polynucleotide sequence comprising a nucleic acid molecule encoding a lysin selected from the group consisting of GN121 (SEQ ID NO: 175), GN217 lysin (SEQ ID NO: 8), GN394 lysin (SEQ ID NO: 48), GN396 lysin (SEQ ID NO: 50), GN408 lysin (SEQ ID NO: 52), GN418 lysin (SEQ ID NO: 54), GN428 (SEQ ID NO: 60), and GN486 (SEQ ID NO: 66) or an active fragment thereof, wherein the lysin or active fragment thereof inhibits P. aeruginosa bacterial growth, reduces a P. aeruginosa bacterial population and/or kills P. aeruginosa in the absence and/or presence of human serum or in the presence of pulmonary surfactant.

In one aspect, the present disclosure is directed to a pharmaceutical composition comprising an isolated lysin and/or a lysin-antimicrobial peptide (AMP) polypeptide construct and a pharmaceutically acceptable carrier,

wherein the isolated lysin comprises at least one of: (i) GN121 (SEQ ID NO: 175), GN123 (SEQ ID NO: 173), GN217 (SEQ ID NO: 8), GN316 variant (SEQ ID NO: 24), GN316 (SEQ ID NO: 22), GN329 (SEQ ID NO: 26), GN333 (SEQ ID NO: 28), GN394 (SEQ ID NO: 48), GN396 (SEQ ID NO: 50), GN408 (SEQ ID NO: 52), GN418 (SEQ ID NO: 54), GN424 (SEQ ID NO: 56), GN425 (SEQ ID NO: 58), GN428 (SEQ ID NO: 60), GN431 (SEQ ID NO: 64), GN486 (SEQ ID NO: 66), GN485 (SEQ ID NO: 68), Lysin PaP2_gp17 (SEQ ID NO: 96), (ii) an active fragment thereof, or (iii) a polypeptide having lysin activity and at least 80% sequence identity with the polypeptide sequence of at least one of SEQ ID NOS: 175, 173, 8, 24, 22, 26, 28, 48, 50, 52, 54, 56, 58, 60, 64, 66, 68, or 96;

wherein the lysin-AMP polypeptide construct comprises: (a) a first component comprising the polypeptide sequence of: (i) a lysin selected from the group consisting of GN76 (SEQ ID NO: 203), GN4 (SEQ ID NO: 74), GN146 (SEQ ID NO: 78), GN14 (SEQ ID NO: 124), GN37 (SEQ ID NO: 84) optionally with a single pI-increasing mutation, GN316 (SEQ ID NO: 22) optionally with a single point mutation, lysin Pap2_gp17 (SEQ ID NO: 96), GN329 (SEQ ID NO: 26), GN424 (SEQ ID NO: 56), GN202 (SEQ ID NO: 118), GN425 (SEQ ID NO: 58), GN428 (SEQ ID NO: 60), GN431 (SEQ ID NO: 64), GN486 (SEQ ID NO: 66), GN333 (SEQ ID NO: 28), GN485 (SEQ ID NO: 68), GN123 (SEQ ID NO: 173) and GN121 (SEQ ID NO: 175); or (ii) a polypeptide having lysin activity and having at least 80% sequence identity with the polypeptide sequence of at least one of SEQ ID NOS: 203, 74, 78, 124, 84, 22, 96, 26, 56, 118, 58, 60, 64, 66, 28, 68, 173 or 175; or (iii) an active fragment of the lysin; and (b) a second component comprising the polypeptide sequence of: (i) at least one antimicrobial peptide (AMP) selected from the group consisting of Chp1 (SEQ ID NO: 133), Chp2 (SEQ ID NO: 70), CPAR39 (SEQ ID NO: 135), Chp3 (SEQ ID NO: 137), Chp4 (SEQ ID NO: 102), Chp6 (SEQ ID NO: 106), Chp7 (SEQ ID NO: 139), Chp8 (SEQ ID NO: 141), Chp9 (SEQ ID NO: 143), Chp10 (SEQ ID NO: 145), Chp11 (SEQ ID NO: 147), Chp12 (SEQ ID NO: 149), Gkh1 (SEQ ID NO: 151), Gkh2 (SEQ ID NO: 90), Unp1 (SEQ ID NO: 153), Ecp1 (SEQ ID NO: 155), Ecp2 (SEQ ID NO: 104), Tma1 (SEQ ID NO: 157), Osp1 (SEQ ID NO: 108), Unp2 (SEQ ID NO: 159), Unp3 (SEQ ID NO: 161), Gkh3 (SEQ ID NO: 163), Unp5 (SEQ ID NO: 165), Unp6 (SEQ ID NO: 167), Spi1 (SEQ ID NO: 169), Spi2 (SEQ ID NO: 171), Ecp3 (SEQ ID NO: 177), Ecp4 (SEQ ID NO: 179), ALCES1 (SEQ ID NO: 181), AVQ206 (SEQ ID NO: 183), AVQ244 (SEQ ID NO: 185), CDL907 (SEQ ID NO: 187), AGT915 (SEQ ID NO: 189), HH3930 (SEQ ID NO: 191), Fen7875 (SEQ ID NO: 193), SBR77 (SEQ ID NO: 195), Bdp1 (SEQ ID NO: 197), LVP1 (SEQ ID NO: 199), Lvp2 (SEQ ID NO: 201), an esculentin fragment (SEQ ID NO: 80), RI12 (SEQ ID NO: 88), TI15 (SEQ ID NO: 94), RI18 (SEQ ID NO: 92), FIRL (SEQ ID NO: 114), a fragment of LPS binding protein (SEQ ID NO: 76), RR12whydro (SEQ ID NO: 110), RI18 peptide derivative (SEQ ID NO: 131) and cationic peptide (SEQ ID NO: 120) or (ii) a polypeptide having AMP activity, wherein the polypeptide is at least 80% identical to at least one of SEQ ID NOS: 133, 70, 135, 137, 102, 106, 139, 141, 143, 145, 147, 149, 151, 90, 153, 155, 104, 157, 108, 159, 161, 163, 165, 167, 169, 171, 177, 179, 181, 183, 185, 187, 189, 191, 193, 195, 197, 199, 201, 80, 88, 94, 92, 114, 76, 110, 131 and 120, wherein the pharmaceutical composition comprises at least one activity selected from inhibiting P. aeruginosa bacterial growth, reducing a P. aeruginosa bacterial population and/or killing P. aeruginosa in the absence and/or presence of human serum or in the presence of pulmonary surfactant.

In another aspect, the present disclosure is directed to a method of treating a bacterial infection caused by a Gram-negative bacteria, wherein the Gram-negative bacteria comprises P. aeruginosa and optionally one or more additional species of Gram-negative bacteria, which method comprises: administering to a subject diagnosed with, at risk for, or exhibiting symptoms of a bacterial infection, a pharmaceutical composition as described herein. In certain embodiments, the bacterial infection is in an organ or tissue in which pulmonary surfactant is present, such as in the lungs or the airways.

In yet another aspect, the present disclosure is directed to a method of preventing or treating a bacterial infection comprising: co-administering to a subject diagnosed with, at risk for, or exhibiting symptoms of a bacterial infection, a combination of a first effective amount of a pharmaceutical composition as described herein, and a second effective amount of an antibiotic suitable for the treatment of a Gram-negative bacterial infection.

In one aspect, the present disclosure is directed to a method for augmenting the efficacy of an antibiotic suitable for the treatment of a Gram-negative bacterial infection, comprising: co-administering the antibiotic in combination with a composition containing an effective amount of an isolated lysin and/or a lysin-antimicrobial peptide (AMP) polypeptide construct,

wherein the isolated lysin comprises at least one of: (i) GN121 (SEQ ID NO: 175), GN123 (SEQ ID NO: 173), GN217 (SEQ ID NO: 8), GN316 variant (SEQ ID NO: 24), GN316 (SEQ ID NO: 22), GN329 (SEQ ID NO: 26), GN333 (SEQ ID NO: 28), GN394 (SEQ ID NO: 48), GN396 (SEQ ID NO: 50), GN408 (SEQ ID NO: 52), GN418 (SEQ ID NO: 54), GN424 (SEQ ID NO: 56), GN425 (SEQ ID NO:58), GN428 (SEQ ID NO: 60), GN431 (SEQ ID NO: 64), GN486 (SEQ ID NO: 66), GN485 (SEQ ID NO: 68), Lysin PaP2_gp17 (SEQ ID NO: 96), or (ii) an active fragment thereof, or (iii) a polypeptide having lysin activity and at least 80% sequence identity with the polypeptide sequence of at least one of SEQ ID NOS: 175, 173, 8, 24, 22, 26, 28, 48, 50, 52, 54, 56, 58, 60, 64, 66, 68, or 96;

wherein the lysin-AMP polypeptide construct comprises: (a) a first component comprising the polypeptide sequence of: (i) a lysin selected from the group consisting of GN76 (SEQ ID NO: 203), GN4 (SEQ ID NO: 74), GN146 (SEQ ID NO: 78), GN14 (SEQ ID NO: 124), GN37 (SEQ ID NO: 84) optionally with a single pI-increasing mutation, GN316 (SEQ ID NO: 22) optionally with a single point mutation, lysin Pap2_gp17 (SEQ ID NO: 96), GN329 (SEQ ID NO: 26), GN424 (SEQ ID NO: 56), GN202 (SEQ ID NO: 118), GN425 (SEQ ID NO: 58), GN428 (SEQ ID NO: 60), GN431 (SEQ ID NO: 64), GN486 (SEQ ID NO: 66), GN333 (SEQ ID NO: 28), GN485 (SEQ ID NO: 68), GN123 (SEQ ID NO: 173) and GN121 (SEQ ID NO: 175); or (ii) a polypeptide having lysin activity and having at least 80% sequence identity with the polypeptide sequence of at least one of SEQ ID NOS: 203, 74, 78, 124, 84, 22, 26, 56, 118, 58, 60, 64, 66, 28, 68, 173 or 175; or (iii) an active fragment of the lysin; and (b) a second component comprising the polypeptide sequence of: (i) at least one antimicrobial peptide (AMP) selected from the group consisting of Chp1 (SEQ ID NO: 133), Chp2 (SEQ ID NO: 70), CPAR39 (SEQ ID NO: 135), Chp3 (SEQ ID NO: 137), Chp4 (SEQ ID NO: 102), Chp6 (SEQ ID NO: 106), Chp7 (SEQ ID NO: 139), Chp8 (SEQ ID NO: 141), Chp9 (SEQ ID NO: 143), Chp10 (SEQ ID NO: 145), Chp11 (SEQ ID NO: 147), Chp12 (SEQ ID NO: 149), Gkh1 (SEQ ID NO: 151), Gkh2 (SEQ ID NO: 90), Unp1 (SEQ ID NO: 153), Ecp1 (SEQ ID NO: 155), Ecp2 (SEQ ID NO: 104), Tma1 (SEQ ID NO: 157), Osp1 (SEQ ID NO: 108), Unp2 (SEQ ID NO: 159), Unp3 (SEQ ID NO: 161), Gkh3 (SEQ ID NO: 163), Unp5 (SEQ ID NO: 165), Unp6 (SEQ ID NO: 167), Spi1 (SEQ ID NO: 169), Spi2 (SEQ ID NO: 171), Ecp3 (SEQ ID NO: 177), Ecp4 (SEQ ID NO: 179), ALCES1 (SEQ ID NO: 181), AVQ206 (SEQ ID NO: 183), AVQ244 (SEQ ID NO: 185), CDL907 (SEQ ID NO: 187), AGT915 (SEQ ID NO: 189), HH3930 (SEQ ID NO: 191), Fen7875 (SEQ ID NO: 193), SBR77 (SEQ ID NO: 195), Bdp1 (SEQ ID NO: 197), LVP1 (SEQ ID NO: 199), Lvp2 (SEQ ID NO: 201), an esculentin fragment (SEQ ID NO: 80), RI12 (SEQ ID NO: 88), TI15 (SEQ ID NO: 94), RI18 (SEQ ID NO: 92), FIRL (SEQ ID NO: 114), a fragment of LPS binding protein (SEQ ID NO: 76), RR12whydro (SEQ ID NO: 110), RI18 peptide derivative (SEQ ID NO: 131) and cationic peptide (SEQ ID NO: 120) or (ii) a polypeptide having AMP activity, wherein the polypeptide is at least 80% identical to at least one of SEQ ID NOS: 133, 70, 135, 137, 102, 106, 139, 141, 143, 145, 147, 149, 151, 90, 153, 155, 104, 157, 108, 159, 161, 163, 165, 167, 169, 171, 177, 179, 181, 183, 185, 187, 189, 191, 193, 195, 197, 199, 201, 80, 88, 94, 92, 114, 76, 110, 131 and 120, wherein the composition comprises at least one activity selected from inhibiting P. aeruginosa bacterial growth, reducing a P. aeruginosa bacterial population and/or killing P. aeruginosa in the absence and/or presence of human serum or in the presence of pulmonary surfactant, and wherein administration of the combination is more effective in inhibiting the growth, or reducing the population, or killing the Gram-negative bacteria in the presence or absence or both in the presence and absence of human serum or in the presence of pulmonary surfactant than administration of either the antibiotic or the lysin or lysin-AMP polypeptide construct individually.

In another aspect, the present disclosure is directed to a method of inhibiting the growth, or reducing the population, or killing of at least one species of Gram-negative bacteria, wherein the at least one species of Gram-negative bacteria is P. aeruginosa and optionally one or more additional species of Gram-negative bacteria, which method comprises: contacting the bacteria with a composition containing an effective amount an isolated lysin and/or a lysin-antimicrobial peptide (AMP) polypeptide construct,

wherein the isolated lysin comprises at least one of: (i) GN121 (SEQ ID NO: 175), GN123 (SEQ ID NO: 173), GN217 (SEQ ID NO: 8), GN316 variant (SEQ ID NO: 24), GN316 (SEQ ID NO: 22), GN329 (SEQ ID NO: 26), GN333 (SEQ ID NO: 28), GN394 (SEQ ID NO: 48), GN396 (SEQ ID NO: 50), GN408 (SEQ ID NO: 52), GN418 (SEQ ID NO: 54), GN424 (SEQ ID NO: 56), GN425 (SEQ ID NO:58), GN428 (SEQ ID NO: 60), GN431 (SEQ ID NO: 64), GN486 (SEQ ID NO: 66), GN485 (SEQ ID NO: 68), Lysin PaP2_gp17 (SEQ ID NO: 96), or (ii) an active fragment thereof, or (iii) a polypeptide having lysin activity and at least 80% sequence identity with the polypeptide sequence of at least one of SEQ ID NOS: 175, 173, 8, 24, 22, 26, 28, 48, 50, 52, 54, 56, 58, 60, 64, 66, 68, or 96;

wherein the lysin-AMP polypeptide construct comprises: (a) a first component comprising the polypeptide sequence of: (i) a lysin selected from the group consisting of GN76 (SEQ ID NO: 203), GN4 (SEQ ID NO: 74), GN146 (SEQ ID NO: 78), GN14 (SEQ ID NO: 124), GN37 (SEQ ID NO: 84) optionally with a single pI-increasing mutation, GN316 (SEQ ID NO: 22) optionally with a single point mutation, lysin Pap2_gp17 (SEQ ID NO: 96), GN329 (SEQ ID NO: 26), GN424 (SEQ ID NO: 56), GN202 (SEQ ID NO: 118), GN425 (SEQ ID NO: 58), GN428 (SEQ ID NO: 60), GN431 (SEQ ID NO: 64), GN486 (SEQ ID NO: 66), GN333 (SEQ ID NO: 28), GN485 (SEQ ID NO: 68), GN123 (SEQ ID NO: 173) and GN121 (SEQ ID NO: 175); or (ii) a polypeptide having lysin activity and having at least 80% sequence identity with the polypeptide sequence of at least one of SEQ ID NOS: 203, 74, 78, 124, 84, 22, 96, 26, 56, 118, 58, 60, 64, 66, 28, 68, 173 or 175; or (iii) an active fragment of the lysin; and (b) a second component comprising the polypeptide sequence of: (i) at least one antimicrobial peptide (AMP) selected from the group consisting of Chp1 (SEQ ID NO: 133), Chp2 (SEQ ID NO: 70), CPAR39 (SEQ ID NO: 135), Chp3 (SEQ ID NO: 137), Chp4 (SEQ ID NO: 102), Chp6 (SEQ ID NO: 106), Chp7 (SEQ ID NO: 139), Chp8 (SEQ ID NO: 141), Chp9 (SEQ ID NO: 143), Chp10 (SEQ ID NO: 145), Chp11 (SEQ ID NO: 147), Chp12 (SEQ ID NO: 149), Gkh1 (SEQ ID NO: 151), Gkh2 (SEQ ID NO: 90), Unp1 (SEQ ID NO: 153), Ecp1 (SEQ ID NO: 155), Ecp2 (SEQ ID NO: 104), Tma1 (SEQ ID NO: 157), Osp1 (SEQ ID NO: 108), Unp2 (SEQ ID NO: 159), Unp3 (SEQ ID NO: 161), Gkh3 (SEQ ID NO: 163), Unp5 (SEQ ID NO: 165), Unp6 (SEQ ID NO: 167), Spi1 (SEQ ID NO: 169), Spi2 (SEQ ID NO: 171), Ecp3 (SEQ ID NO: 177), Ecp4 (SEQ ID NO: 179), ALCES1 (SEQ ID NO: 181), AVQ206 (SEQ ID NO: 183), AVQ244 (SEQ ID NO: 185), CDL907 (SEQ ID NO: 187), AGT915 (SEQ ID NO: 189), HH3930 (SEQ ID NO: 191), Fen7875 (SEQ ID NO: 193), SBR77 (SEQ ID NO: 195), Bdp1 (SEQ ID NO: 197), LVP1 (SEQ ID NO: 199), Lvp2 (SEQ ID NO: 201), an esculentin fragment (SEQ ID NO: 80), RI12 (SEQ ID NO: 88), TI15 (SEQ ID NO: 94), RI18 (SEQ ID NO: 92), FIRL (SEQ ID NO: 114), a fragment of LPS binding protein (SEQ ID NO: 76), RR12whydro (SEQ ID NO: 110), RI18 peptide derivative (SEQ ID NO: 131) and cationic peptide (SEQ ID NO: 120) or (ii) a polypeptide having AMP activity, wherein the polypeptide is at least 80% identical to at least one of SEQ ID NOS: 133, 70, 135, 137, 102, 106, 139, 141, 143, 145, 147, 149, 151, 90, 153, 155, 104, 157, 108, 159, 161, 163, 165, 167, 169, 171, 177, 179, 181, 183, 185, 187, 189, 191, 193, 195, 197, 199, 201, 80, 88, 94, 92, 114, 76, 110, 131 and 120, and wherein the composition comprises at least one activity selected from inhibiting P. aeruginosa bacterial growth, reducing a P. aeruginosa bacterial population and/or killing P. aeruginosa in the absence and/or presence of human serum or in the presence of pulmonary surfactant.

### BRIEF DESCRIPTION OF THE DRAWINGS

FIG. 1 depicts three-dimensional models predicted by I-Tasser for structures of Chlamydia phage peptide (Chp) family members Chp1, Chp2, Chp4, Chp5, Chp6, Chp7, Ecp1, Ecp2, and Osp1. The human innate immune effector peptide LL-37 is included for comparison. Alpha helical structures are evident, and the top terminal is generally the N-terminal.

FIG. 2A is a graph showing the percent relative fluorescence unit (RFU) over time for P. aeruginosa in the presence of N-phenyl-1-napthylamine (NPN) and buffer, GN121, or GN351, as described in Example 6.

FIG. 2B is a graph showing the percent RFU over time for P. aeruginosa in the presence of NPN and buffer, GN428, or GN370, as described in Example 6.

FIG. 3 is a series of photomicrographs showing microscopic analysis (×2000 magnification) of Pseudomonas aeruginosa strain 1292 treated for 15 minutes with GN121 (10 μg/mL) or a buffer control (“untreated”) in 100% human serum. Samples were stained using the Live/Dead Cell Viability Kit (ThermoFisher) and examined by both differential interference contrast (DIC) and fluorescence microscopy. The photomicrographs show an absence of dead bacteria in the untreated row and a reduction of live bacteria in the treated row, as described in Example 7.

FIGS. 4A-4E show the fold change in GN lysin and Ciprofloxacin needed to achieve a Minimal Inhibitory Concentration (MIC) for P. aeruginosa (strain WC-452) over 21 day serial passage as described in Example 9: GN121 (FIG. 4A), GN351 (FIG. 4B), GN370 (FIG. 4C), GN428 (FIG. 4D) and Ciprofloxacin (FIG. 4E).

### DETAILED DESCRIPTION

### Definitions

As used herein, the following terms and cognates thereof shall have the following meanings unless the context clearly indicates otherwise:

“Carrier” refers to a solvent, additive, excipient, dispersion medium, solubilizing agent, coating, preservative, isotonic and absorption delaying agent, surfactant, propellant, diluent, vehicle and the like with which an active compound is administered. Such carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil, and the like.

“Pharmaceutically acceptable carrier” refers to any and all solvents, additives, excipients, dispersion media, solubilizing agents, coatings, preservatives, isotonic and absorption delaying agents, surfactants, propellants, diluents, vehicles and the like that are physiologically compatible. The carrier(s) must be “acceptable” in the sense of not being deleterious to the subject to be treated in amounts typically used in medicaments. Pharmaceutically acceptable carriers are compatible with the other ingredients of the composition without rendering the composition unsuitable for its intended purpose. Furthermore, pharmaceutically acceptable carriers are suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers or excipients include any of the standard pharmaceutical carriers such as phosphate buffered saline solutions, water, and emulsions such as oil/water emulsions and microemulsions. Suitable pharmaceutical carriers are described, for example, in Remington's Pharmaceutical Sciences by E. W. Martin, 18th Edition. The pharmaceutically acceptable carrier may be a carrier that does not exist in nature.

“Bactericidal” or “bactericidal activity” refers to the property of causing the death of bacteria or capable of killing bacteria to an extent of at least a 3-log 10 (99.9%) or better reduction among an initial population of bacteria over an 18-24 hour period.

“Bacteriostatic” or “bacteriostatic activity” refers to the property of inhibiting bacterial growth, including inhibiting growing bacterial cells, thus causing a 2-log 10 (99%) or better and up to just under a 3-log reduction among an initial population of bacteria over an 18-24 hour period.

“Antibacterial” refers to both bacteriostatic and bactericidal agents.

“Antibiotic” refers to a compound having properties that have a negative effect on bacteria, such as lethality or reduction of growth. An antibiotic can have a negative effect on Gram-positive bacteria, Gram-negative bacteria, or both. By way of example, an antibiotic can affect cell wall peptidoglycan biosynthesis, cell membrane integrity, or DNA or protein synthesis in bacteria. Nonlimiting examples of antibiotics active against Gram-negative bacteria include cephalosporins, such as ceftriaxone-cefotaxime, ceftazidime, cefepime, cefoperazone, and ceftobiprole; fluoroquinolones such as ciprofloxacin and levofloxacin; aminoglycosides such as gentamicin, tobramycin, and amikacin; piperacillin, ticarcillin, imipenem, meropenem, doripenem, broad spectrum penicillins with or without beta-lactamase inhibitors, rifampicin, polymyxin B, and colistin.

“Drug resistant” generally refers to a bacterium that is resistant to the antibacterial activity of a drug. When used in certain ways, drug resistance may specifically refer to antibiotic resistance. In some cases, a bacterium that is generally susceptible to a particular antibiotic can develop resistance to the antibiotic, thereby becoming a drug resistant microbe or strain. A “multi-drug resistant” (“MDR”) pathogen is one that has developed resistance to at least two classes of antimicrobial drugs, each used as monotherapy. For example, certain strains of S. aureus have been found to be resistant to several antibiotics including methicillin and/or vancomycin (Antibiotic Resistant Threats in the United States, 2013, U.S. Department of Health and Services, Centers for Disease Control and Prevention). One skilled in the art can readily determine if a bacterium is drug resistant using routine laboratory techniques that determine the susceptibility or resistance of a bacterium to a drug or antibiotic.

“Effective amount” refers to an amount which, when applied or administered in an appropriate frequency or dosing regimen, is sufficient to prevent, reduce, inhibit, or eliminate bacterial growth or bacterial burden or to prevent, reduce, or ameliorate the onset, severity, duration, or progression of the disorder being treated (for example, Gram-negative bacterial pathogen growth or infection), prevent the advancement of the disorder being treated, cause the regression of the disorder being treated, or enhance or improve the prophylactic or therapeutic effect(s) of another therapy, such as antibiotic or bacteriostatic therapy.

“Co-administer” refers to the administration of two agents, such as a lysin or lysin-AMP polypeptide and an antibiotic or any other antibacterial agent, in a sequential manner, as well as administration of these agents in a substantially simultaneous manner, such as in a single mixture/composition or in doses given separately, but nonetheless administered substantially simultaneously to the subject, for example at different times in the same day or 24-hour period. Such co-administration of two agents, such as a lysin or lysin-AMP polypeptide with one or more additional antibacterial agents can be provided as a continuous treatment lasting up to days, weeks, or months. Additionally, depending on the use, the co-administration need not be continuous or coextensive. For example, if the use were as a topical antibacterial agent to treat, e.g., a bacterial ulcer or an infected diabetic ulcer, a lysin or lysin-AMP polypeptide could be administered only initially within 24 hours of an additional antibiotic, and then the additional antibiotic use may continue without further administration of the lysin or lysin-AMP polypeptide.

“Subject” refers to a mammal, a plant, a lower animal, a single cell organism, or a cell culture. For example, the term “subject” is intended to include organisms, e.g., prokaryotes and eukaryotes, which are susceptible to or afflicted with bacterial infections, for example Gram-positive or Gram-negative bacterial infections. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or susceptible to infection by Gram-negative bacteria, whether such infection be systemic, topical or otherwise concentrated or confined to a particular organ or tissue.

“Polypeptide” is used herein interchangeably with the term “peptide” or “protein” and refers to a polymer made from amino acid residues and generally having at least about 30 amino acid residues. The term includes not only polypeptides in isolated form, but also active fragments and derivatives thereof. The term “polypeptide” also encompasses fusion proteins or fusion polypeptides comprising a lysin or AMP as described herein and maintaining, for example a lytic function. Depending on context, a polypeptide can be a naturally occurring polypeptide or a recombinant, engineered, or synthetically produced polypeptide. A particular lysin polypeptide, for example, can be, for example, derived or removed from a native protein by enzymatic or chemical cleavage, or can be prepared using conventional peptide synthesis techniques (e.g., solid phase synthesis) or molecular biology techniques (such as those disclosed in Sambrook, J. et al., Molecular Cloning: A Laboratory Manual, Cold Spring Harbor Press, Cold Spring Harbor, N.Y. (1989)) or can be strategically truncated or segmented yielding active fragments, maintaining, e.g., lytic activity against the same or at least one common target bacterium.

“Fusion polypeptide” refers to an expression product resulting from the fusion of two or more nucleic acid segments, resulting in a fused expression product typically having two or more domains or segments, which typically have different properties or functionality. In a more particular sense, the term “fusion polypeptide” may also refer to a polypeptide or peptide comprising two or more heterologous polypeptides or peptides covalently linked, either directly or via an amino acid or peptide linker. The polypeptides forming the fusion polypeptide are typically linked C-terminus to N-terminus, although they can also be linked C-terminus to C-terminus, N-terminus to N-terminus, or N-terminus to C-terminus. The term “fusion polypeptide” can be used interchangeably with the term “fusion protein.” The open-ended expression “a polypeptide comprising” a certain structure includes larger molecules than the recited structure, such as fusion polypeptides.

“Heterologous” refers to nucleotide, peptide, or polypeptide sequences that are not naturally contiguous. For example, in the context of the present disclosure, the term “heterologous” can be used to describe a combination or fusion of two or more peptides and/or polypeptides wherein the fusion peptide or polypeptide is not normally found in nature, such as for example a lysin or active fragment thereof and an antimicrobial peptide, including a cationic and/or a polycationic peptide, an amphipathic peptide, a sushi peptide (Ding et al. Cell Mol Life Sci., 65(7-8): 1202-19 (2008)), a defensin peptide (Ganz, T. Nature Reviews Immunology 3, 710-720 (2003)), a hydrophobic peptide, which may have enhanced lytic activity.

“Active fragment” refers to a portion of a polypeptide that retains one or more functions or biological activities of the isolated polypeptide from which the fragment was taken, for example bactericidal activity against one or more Gram-negative bacteria.

“Amphipathic peptide” refers to a peptide having both hydrophilic and hydrophobic functional groups. In certain embodiments, secondary structure may place hydrophobic and hydrophilic amino acid residues at opposite sides (e.g., inner side vs outer side when the peptide is in a solvent, such as water) of an amphipathic peptide. These peptides may in certain embodiments adopt a helical secondary structure, such as an alpha-helical secondary structure.

“Cationic peptide” refers to a peptide having a high percentage of positively charged amino acid residues. In certain embodiments, a cationic peptide has a pKa-value of 8.0 or greater. The term “cationic peptide” in the context of the present disclosure also encompasses polycationic peptides that are synthetically produced peptides composed of mostly positively charged amino acid residues, such as lysine (Lys) and/or arginine (Arg) residues. The amino acid residues that are not positively charged can be neutrally charged amino acid residues, negatively charged amino acid residues, and/or hydrophobic amino acid residues.

“Hydrophobic group” refers to a chemical group such as an amino acid side chain that has low or no affinity for water molecules but higher affinity for oil molecules. Hydrophobic substances tend to have low or no solubility in water or aqueous phases and are typically apolar but tend to have higher solubility in oil phases. Examples of hydrophobic amino acids include glycine (Gly), alanine (Ala), valine (Val), Leucine (Leu), isoleucine (Ile), proline (Pro), phenylalanine (Phe), methionine (Met), and tryptophan (Trp).

“Augmenting” refers to a degree of activity of an agent, such as antimicrobial activity, that is higher than it would be otherwise. “Augmenting” encompasses additive as well as synergistic (superadditive) effects.

“Synergistic” or “superadditive” refers to a beneficial effect brought about by two substances in combination that exceeds the sum of the effects of the two agents working independently. In certain embodiments the synergistic or superadditive effect significantly, i.e., statistically significantly, exceeds the sum of the effects of the two agents working independently. One or both active ingredients may be employed at a sub-threshold level, i.e., a level at which if the active substance is employed individually produces no or a very limited effect. The effect can be measured by assays such as the checkerboard assay, described here.

“Treatment” refers to any process, action, application, therapy, or the like, wherein a subject, such as a human being, is subjected to medical aid with the object of curing a disorder, eradicating a pathogen, or improving the subject's condition, directly or indirectly. Treatment also refers to reducing incidence, alleviating symptoms, eliminating recurrence, preventing recurrence, preventing incidence, reducing the risk of incidence, improving symptoms, improving prognosis, or combinations thereof. “Treatment” may further encompass reducing the population, growth rate, or virulence of a bacteria in the subject and thereby controlling or reducing a bacterial infection in a subject or bacterial contamination of an organ, tissue, or environment. Thus “treatment” that reduces incidence may, for example, be effective to inhibit growth of at least one Gram-negative bacterium in a particular milieu, whether it be a subject or an environment. On the other hand, “treatment” of an already established infection refers to inhibiting the growth, reducing the population, killing, including eradicating, a Gram-negative bacteria responsible for an infection or contamination.

“Preventing” refers to the prevention of the incidence, recurrence, spread, onset or establishment of a disorder such as a bacterial infection. It is not intended that the present disclosure be limited to complete prevention or to prevention of establishment of an infection. In some embodiments, the onset is delayed, or the severity of a subsequently contracted disease or the chance of contracting the disease is reduced, and such constitute examples of prevention.

“Contracted diseases” refers to diseases manifesting with clinical or subclinical symptoms, such as the detection of fever, sepsis, or bacteremia, as well as diseases that may be detected by growth of a bacterial pathogen (e.g., in culture) when symptoms associated with such pathology are not yet manifest.

The term “derivative” in the context of a peptide or polypeptide or active fragments thereof is intended to encompass, for example, a polypeptide modified to contain one or more chemical moieties other than an amino acid that do not substantially adversely impact or destroy the polypeptide's activity (e.g., lytic activity). The chemical moiety can be linked covalently to the peptide, e.g., via an amino terminal amino acid residue, a carboxy terminal amino acid residue, or at an internal amino acid residue. Such modifications may be natural or non-natural. In certain embodiments, a non-natural modification may include the addition of a protective or capping group on a reactive moiety, addition of a detectable label, such as antibody and/or fluorescent label, addition or modification of glycosylation, or addition of a bulking group such as PEG (pegylation) and other changes known to those skilled in the art. In certain embodiments, the non-natural modification may be a capping modification, such as N-terminal acetylations and C-terminal amidations. Exemplary protective groups that may be added to lysin polypeptides or AMPs include, but are not limited to, t-Boc and Fmoc. Commonly used fluorescent label proteins such as, but not limited to, green fluorescent protein (GFP), red fluorescent protein (RFP), cyan fluorescent protein (CFP), yellow fluorescent protein (YFP), and mCherry, are compact proteins that can be bound covalently or noncovalently to a polypeptide or fused to a polypeptide without interfering with normal functions of cellular proteins. In certain embodiments, a polynucleotide encoding a fluorescent protein may be inserted upstream or downstream of the lysin or AMP polynucleotide sequence. This will produce a fusion protein (e.g., Lysin Polypeptide::GFP) that does not interfere with cellular function or function of a polypeptide to which it is attached. Polyethylene glycol (PEG) conjugation to proteins has been used as a method for extending the circulating half-life of many pharmaceutical proteins. Thus, in the context of polypeptide derivatives, such as lysin polypeptide derivatives, the term “derivative” encompasses polypeptides, such as lysin polypeptides, chemically modified by covalent attachment of one or more PEG molecules. It is anticipated that lysin polypeptides, such as pegylated lysins, will exhibit prolonged circulation half-life compared to the unpegylated polypeptides, while retaining biological and therapeutic activity.

“Percent amino acid sequence identity” refers to the percentage of amino acid residues in a candidate sequence that are identical with the amino acid residues in the reference polypeptide sequence, such as a lysin polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for example, using publicly available software such as BLAST or software available commercially, for example from DNASTAR. Two or more polypeptide sequences can be anywhere from 0-100% identical, or any integer value there between. In the context of the present disclosure, two polypeptides are “substantially identical” when at least 80% of the amino acid residues (such as at least about 85%, at least about 90%, at least about 92.5%, at least about 95%, at least about 98%, or at least about 99%) are identical. The term “percent (%) amino acid sequence identity” as described herein applies to peptides as well. Thus, the term “substantially identical” will encompass mutated, truncated, fused, or otherwise sequence-modified variants of isolated lysin polypeptides and peptides and AMPs described herein, and active fragments thereof, as well as polypeptides with substantial sequence identity (e.g., at least 80%, at least 85%, at least 90%, at least 92.5%, at least 95%, at least 98%, or at least 99% identity as measured for example by one or more methods referenced above) as compared to the reference (wild type or other intact) polypeptide.

As used herein, two amino acid sequences are “substantially homologous” when at least about 80% of the amino acid residues (such as at least about 85%, at least about 90%, at least about 92.5%, at least about 95%, at least about 98%, or at least about 99%) are identical, or represent conservative substitutions. The sequences of the polypeptides of the present disclosure are substantially homologous when one or more, such as up to 10%, up to 15%, or up to 20% of the amino acids of the polypeptide, such as the lysin, AMP, and/or fusion polypeptides described herein, are substituted with a similar or conservative amino acid substitution, and wherein the resulting peptides have at least one activity (e.g., antibacterial effect) and/or bacterial specificities of the reference polypeptide, such as the lysin, AMP, and/or fusion polypeptides described herein.

As used herein, a “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

“Inhalable composition” refers to pharmaceutical compositions of the present disclosure that are formulated for direct delivery to the respiratory tract during or in conjunction with routine or assisted respiration (e.g., by intratracheobronchial, pulmonary, and/or nasal administration), including, but not limited to, atomized, nebulized, dry powder, and/or aerosolized formulations.

“Biofilm” refers to bacteria that attach to surfaces and aggregate in a hydrated polymeric matrix that may be comprised of bacterial- and/or host-derived components. A biofilm is an aggregate of microorganisms in which cells adhere to each other on a biotic or abiotic surface. These adherent cells are frequently embedded within a matrix comprised of, but not limited to, extracellular polymeric substance (EPS). Biofilm EPS, which is also referred to as slime (although not everything described as slime is a biofilm) or plaque, is a polymeric conglomeration generally composed of extracellular DNA, proteins, and polysaccharides.

“Preventing biofilm formation” refers to the prevention of the incidence, recurrence, spread, onset or establishment of a biofilm. It is not intended that the present disclosure be limited to complete prevention or to prevention of establishment of biofilm. In some embodiments, the onset of a biofilm is delayed, or the establishment of a biofilm is reduced or the chance of formation of a new biofilm is reduced, and such constitute examples of prevention of a biofilm. Further, prevention of a biofilm may be due to any mechanism including 1) effectively killing planktonic bacteria; 2) killing “persister” bacterial cells in suspensions, i.e., bacteria that are metabolically inactive, tolerant of antibiotics, and highly associated with biofilm formation; and/or 3) preventing “aggregation”, i.e., the ability of bacteria to attach to one another via proteins or polysaccharides.

“Eradication” in reference to a biofilm includes 1) effectively killing bacteria in a biofilm including persister bacterial cells in the biofilm and, optionally 2) effectively destroying and/or damaging the biofilm matrix.

“Disruption” in reference to a biofilm refers to a mechanism that falls between prevention and eradication. A biofilm, which is disrupted, may be “opened”, or otherwise damaged, thus permitting, e.g., an antibiotic, to more readily penetrate the biofilm and kill the bacteria.

“Suitable” in the context of an antibiotic being suitable for use against certain bacteria refers to an antibiotic that was found to be effective against those bacteria even if resistance subsequently developed.

“Outer Membrane” or “OM” refers to a feature of Gram-negative bacteria. The outer membrane is comprised of a lipid bilayer with an internal leaflet of phospholipids and an external amphiphilic leaflet largely consisting of lipopolysaccharide (LPS). The LPS has three main sections: a hexa-acylated glucosamine-based phospholipid called lipid A, a polysaccharide core and an extended, external polysaccharide chain called O-antigen. The OM presents a non-fluid continuum stabilized by three major interactions, including: i) the avid binding of LPS molecules to each other, especially if cations are present to neutralize phosphate groups; ii) the tight packing of largely saturated acyl chains; and iii) hydrophobic stacking of the lipid A moiety. The resulting structure is a barrier for both hydrophobic and hydrophilic molecules. Below the OM, the peptidoglycan forms a thin layer that is very sensitive to hydrolytic cleavage—unlike the peptidoglycan of Gram-negative bacteria which is 30-100 nanometers (nm) thick and consists of up to 40 layers, the peptidoglycan of Gram-negative bacteria is only 2-3 nm thick and consists of only 1-3 layers.

### Polypeptides

Lysins, Variant Lysins, Active Fragments Thereof or Derivatives

The present disclosure is directed to isolated polypeptides comprising lysins, variant lysins, active fragments thereof or derivatives. In some embodiments, the isolated polypeptides comprising the lysins, variant lysins, active fragments thereof or derivatives are combined with antimicrobial peptides (“AMPs”) to form a lysin-AMP polypeptide construct, wherein the lysin-AMP polypeptide construct has lysin activity. As used herein “lysin activity” encompasses the ability of a lysin to kill bacteria (e.g., P. aeruginosa), reduce the population of bacteria or inhibit bacterial growth (e.g., by penetrating the outer membrane of a Gram-negative bacteria), optionally in the presence of human serum or pulmonary surfactant. Lysin activity also encompasses the ability to remove or reduce a biofilm and/or the ability to reduce the minimum inhibitory concentration (MIC) of an antibiotic, optionally in the presence of human serum or pulmonary surfactant.

In some embodiments, the present isolated polypeptides comprising lysins, variant lysins, active fragments thereof or derivatives thereof are capable of penetrating the outer membrane of Gram-negative bacteria. Without being limited by theory, after penetration of the outer membrane, the present isolated polypeptides comprising lysins, variant lysins, active fragments thereof or derivatives thereof can degrade peptidoglycan, a major structural component of the bacterial cell wall, resulting in e.g., cell lysis or non-lethal damage that inhibits bacterial growth. In some embodiments, the present isolated polypeptides comprising lysins, variant lysins, active fragments thereof or derivatives disclosed herein contain positively charged (and amphipathic)N- and/or C-terminal α-helical domains that facilitate binding to the anionic outer membrane of a Gram-negative bacteria to effect translocation into the sub-adjacent peptidoglycan.

The ability of a lysin to penetrate an outer membrane of a Gram-negative bacteria may be assessed by any method known in the art, such as described in WO 2017/049233, which is herein incorporated by reference in its entirety. For example, the lysin may be incubated with Gram-negative bacteria and a hydrophobic compound. Most Gram-negative bacteria are strongly resistant to hydrophobic compounds, due to the presence of the outer membrane and, thus, do not allow the uptake of hydrophobic agents such as 1-N-phenylnaphthylamine (NPN), crystal violet, or 8-anilino-1-naphthalenesulfonic acid (ANS). NPN, for example, fluoresces strongly under hydrophobic conditions and weakly under aqueous conditions. Accordingly, NPN fluorescence can be used as a measurement of the outer membrane permeability.

More particularly, the ability of a lysin to penetrate an outer wall may be assessed by incubating, e.g., NPN with a Gram-negative bacteria, e.g., P. aeruginosa strain PA01, in the presence of the lysin to be tested for activity. A higher induction of fluorescence in comparison to the fluorescence emitted in the absence of a lysin (negative control) indicates outer membrane penetration. In addition, fluorescence induction can be compared to that of established permeabilizing agents, such as EDTA (ethylene diamine tetraacetate) or an antibiotic such as an antibiotic of last resort used in the treatment of P. aeruginosa, i.e., Polymyxin B (PMB) to assess the level of outer membrane permeability.

In some embodiments, the present isolated polypeptides comprising lysins, variant lysins, active fragments thereof or derivatives exhibit lysin activity in the presence and/or absence of human serum. Suitable methods for assessing the activity of a lysin in human serum are known in the art and described in the examples. Briefly, a MIC value (i.e., the minimum concentration of peptide sufficient to suppress at least 80% of the bacterial growth compared to control) may be determined for a lysin and compared to, e.g., a parent lysin or compound inactive in human serum, e.g., T4 phage lysozyme or artilysin GN126. T4 phage lysozyme is commercially available, e.g. from Sigma-Aldrich, Inc. GN126 corresponds to Art-175, which is described in the literature and is obtained by fusing AMP SMAP-29 to GN lysin KZ144. See Briers et al. 2014, Antimicrob, Agents Chemother. 58:3774-3784, which is herein incorporated by reference in its entirety.

More particularly MIC values for a lysin may be determined against e.g., the laboratory P. aeruginosa strain PA01, in e.g., Mueller-Hinton broth, Mueller-Hinton broth supplemented with human serum, CAA as described herein, which includes physiological salt concentrations, and CAA supplemented with human serum. The use of PA01 enables testing in the presence of elevated serum concentrations since unlike most clinical isolates, PA01 is insensitive to the antibacterial activity of human blood matrices.

In some embodiments, the present isolated polypeptides comprising lysins, variant lysins, active fragments thereof or derivatives are capable of reducing a biofilm. Methods for assessing the Minimal Biofilm Eradicating Concentration (MBEC) of a lysin or AMP may be determined using a variation of the broth microdilution MIC method with modifications (See Ceri et al. 1999. J. Clin Microbial. 37:1771-1776, which is herein incorporated by reference in its entirety and Schuch et al., 2017, Antimicrob. Agents Chemother. 61, pages 1-18, which is herein incorporated by reference in its entirety.) In this method, fresh colonies of e.g., a P. aeruginosa strain, such as ATCC 17647, are suspended in medium, e.g., phosphate buffer solution (PBS) diluted e.g., 1:100 in TSBg (tryptic soy broth supplemented with 0.2% glucose), added as e.g., 0.15 ml aliquots, to a Calgary Biofilm Device (96-well plate with a lid bearing 96 polycarbonate pegs; Innovotech Inc.) and incubated e.g., 24 hours at 37° C. Biofilms are then washed and treated with e.g., a 2-fold dilution series of the lysin in TSBg at e.g., 37° C. for 24 hours. After treatment, wells are washed, air-dried at e.g., 37° C. and stained with e.g., 0.05% crystal violet for 10 minutes. After staining, the biofilms are destained in e.g., 33% acetic acid and the OD600 of e.g., extracted crystal violet is determined. The MBEC of each sample is the minimum lysin concentration required to remove >95% of the biofilm biomass assessed by crystal violet quantitation.

In some embodiments, the present isolated polypeptides comprising lysins, variant lysins, active fragments thereof or derivatives reduce the minimum inhibitory concentration (MIC) of an antibiotic needed to inhibit bacteria in the presence and/or absence of human serum or in the presence of pulmonary surfactant. Any known method to assess MIC may be used. In some embodiments, a checkerboard assay is used to determine the effect of a lysin on antibiotic concentration. The checkerboard assay is based on a modification of the CLSI method for MIC determination by broth microdilution (See Clinical and Laboratory Standards Institute (CLSI), CLSI. 2015. Methods for Dilution Antimicrobial Susceptibility Tests for Bacteria That Grow Aerobically; Approved Standard-10th Edition. Clinical and Laboratory Standards Institute, Wayne, Pa., which is herein incorporated by reference in its entirety and Ceri et al. 1999. J. Clin. Microbiol. 37: 1771-1776, which is also herein incorporated by reference in its entirety).

Checkerboards are constructed by first preparing columns of e.g., a 96-well polypropylene microtiter plate, wherein each well has the same amount of antibiotic diluted 2-fold along the horizontal axis. In a separate plate, comparable rows are prepared in which each well has the same amount of lysin diluted e.g., 2-fold along the vertical axis. The lysin and antibiotic dilutions are then combined, so that each column has a constant amount of antibiotic and doubling dilutions of lysin, while each row has a constant amount of lysin and doubling dilutions of antibiotic. Each well thus has a unique combination of lysin and antibiotic. Bacteria are added to the drug combinations at concentrations of 1×10⁵ CFU/ml in CAA, for example, with or without human serum or pulmonary surfactant (such as Survanta®). The MIC of each drug, alone and in combination, is then recorded after e.g., 16 hours at 37° C. in ambient air. Summation fractional inhibitory concentrations (ΣFICs) are calculated for each drug and the minimum 1FIC value (ΣFICmin) is used to determine the effect of the lysin/antibiotic combination.

In some embodiments, the present lysins and lysin-AMP polypeptide constructs are able to synergize with antibiotics, such as imipenem and meropenem, and drive the resensitization of gram-negative bacteria including MDR organisms, such as carbapenem-resistant P. aeruginosa. Such resensitization may be determined by combining the present lysins or lysin-AMP polypeptide constructs with an antibiotic in a checkerboard assay as described herein. Antibiotic-resistant bacteria, such as carbapenem-resistant P. aeruginosa, are added to the lysin or lysin-AMP polypeptide construct combination. Generally resensitization occurs in synergistic combinations in which the antibiotic MIC values fall below established breakpoints, e.g., a MIC value of ≤2 for antibiotic sensitive bacteria, a MIC value of 4 for intermediately sensitive bacteria and a MIC value of ≥8 for antibiotic resistant bacteria, e.g. carbapenem-resistant isolates. See Clinical and Laboratory Standards Institute (CLSI), CLSI. 2019. M100 Performance Standards for Antimicrobial Susceptibility Testing; 29th Edition. Clinical and Laboratory Standards Institute, Wayne, Pa., which is herein incorporated by reference in its entirety.

In some embodiments, the present isolated polypeptides comprising lysins, variant lysins, active fragments thereof or derivatives show low toxicity against erythrocytes. Any methodology known in the art may be used to assess the potential for hemolytic activity of the present isolated polypeptides comprising lysins, variant lysins, active fragments thereof or derivatives.

Examples of suitable lysins of the present disclosure, particularly for use with the lysin-AMP polypeptide constructs described herein, include the GN316 lysin obtained from Klebsiella phage 0507-KN2-1 (NCBI Reference Sequence: YP_008531963.1, SEQ ID NO: 22), Lysin PaP2_gp17 obtained from Pseudomonas phage (NCBI Reference Sequence: YP_024745.1, SEQ ID NO: 96), GN333 obtained from Delftia sp. (NCBI Reference Sequence: WP 016064791.1, SEQ ID NO: 28), GN424 obtained from Burkholderia pseudomultivorans (NCBI Reference Sequence: WP_060250996.1, SEQ ID NO: 56), GN425 lysin obtained from Pseudomonas flexibilis (NCBI Reference Sequence: WP_039605935.1, SEQ ID NO: 58), GN428 obtained from Escherichia virus CBA120 (NCBI Reference Sequence: YP_004957781.1, SEQ ID NO: 60), GN431 obtained from Dickeya phage phiD3 (NCBI Reference Sequence: AIM51349.1, SEQ ID NO: 64), GN485 obtained from Erwinia sp. Leaf5 (NCBI Reference Sequence: WP_056233282.1, SEQ ID NO: 68) and GN123 obtained from Pseudomonas phage PhiPA3 (NCBI Reference Sequence: YP_009217242.1, SEQ ID NO: 173).

The above described lysins were identified by bioinformatics techniques. Although some of the identified sequences had been annotated as putative peptidoglycan binding proteins, no function had been previously definitively attributed to polypeptides having these sequences. The inventors have surprisingly recognized that the above-identified sequences are suitable for use as antibacterial agents, in particular, against Gram-negative bacteria as described in the examples.

Additional examples of suitable lysins of the present disclosure, particularly those for use with the present lysin-AMP polypeptide constructs, include the GN76 lysin obtained from Acinetobacter phage vB_AbaP_CEB1 (NCBI Reference Sequence ALC76575.1, SEQ ID NO: 203 GenBank: ALC76575.1), the GN4 lysin obtained from Pseudomonas phage PAJU2 (NCBI Reference Sequence YP_002284361.1, SEQ ID NO: 74), the GN14 lysin obtained from Pseudomonas phage Lull (NCBI Reference Sequence YP_006382555.1, SEQ ID NO: 124) and the GN37 lysin obtained from Micavibrio aeruginosavorus (NCBI Reference Sequence WP 014102102.1, SEQ ID NO: 84). Each of the foregoing lysins is also disclosed in WO 2017/049233, which is herein incorporated by reference in its entirety.

In some embodiments, the present isolated polypeptides comprise a lysin variant, e.g., a lysin containing one or more insertions, deletions and/or amino acid substitutions in comparison to a reference lysin polypeptide, e.g., a naturally occurring lysin or a parent lysin, which itself is a variant lysin. In some embodiments, an isolated polypeptide sequence comprising a variant lysin, active fragment thereof or derivative has at least 80%, such as at least 85%, such as at least 90%, such as at least 95%, such as at least 98% or such as at least 99% sequence identity with the reference lysin and/or active fragment thereof described herein.

The lysin variants of the present disclosure typically retain one or more functional or biological activities of a reference lysin. In some embodiments, the modification improves the antibacterial activity of the lysin. Typically, the lysin variant has improved in vitro antibacterial activity (e.g., in buffer and/or media) in comparison to the reference lysin. In other embodiments, the lysin variant has improved in vivo antibacterial activity (e.g., in an animal infection model). In some embodiments, the modification improves the antibacterial activity of the lysin in the absence and/or presence of human serum. In some embodiments, the modification improves the antibacterial activity of the lysin in the presence of pulmonary surfactant.

Suitable variant lysins, particularly those for use in the present lysin-AMP polypeptide constructs, include the GN146 lysin (SEQ ID NO: 78), GN156 lysin (SEQ ID NO: 126), the GN202 lysin (SEQ ID NO: 118) and GN121 lysin (SEQ ID NO: 175). Each of the foregoing lysins is also disclosed in U.S. Provisional Application No. 62/597,577, which was filed on Dec. 12, 2017 and U.S. Provisional Application No. 62/721,969, which was filed on 23 Aug. 2018, and is herein incorporated by reference in its entirety. The lysins described in U.S. Provisional Application No. 62/721,969, typically, are modified in reference to their naturally occurring counterpart to enhance the activity of the lysin in serum, e.g., by introducing amino acid substitutions and/or introducing amino acid fragments from larger antimicrobial peptides. For example, the amino acid sequence GPRRPRRPGRRAPV (residues 1-14 of SEQ ID NO: 126) described by Daniels and Scepartz, 2007, J. Am. Chem. Soc. 129:14578-14579, which is herein incorporated by reference in its entirety, is introduced, for example, at the N terminus of GN4 (SEQ ID NO: 74), to generate GN156 (SEQ ID NO: 126), a non-naturally occurring lysin-AMP polypeptide construct.

In some embodiments, the variant lysins are obtained by modifying a reference lysin to include a modification resulting in a change in the overall isoelectric point (pI) of the lysin, i.e., the pH at which a molecule has a net neutral charge by, for example, incorporating a single pI-increasing mutation, such as a single point mutation, into a reference lysin. Suitable reference lysin polypeptides include a lysin selected from the group consisting of GN76 (SEQ ID NO: 203), GN4 (SEQ ID NO: 74), GN146 (SEQ ID NO: 78), GN14 (SEQ ID NO: 124), GN37 (SEQ ID NO: 84) GN316 (SEQ ID NO: 22) lysin Pap2_gp17 (SEQ ID NO: 96), GN329 (SEQ ID NO: 26), GN424 (SEQ ID NO: 56), GN202 (SEQ ID NO: 118), GN425 (SEQ ID NO: 58), GN428 (SEQ ID NO: 60), GN431 (SEQ ID NO: 64), GN486 (SEQ ID NO: 66), GN333 (SEQ ID NO: 28) GN485 (SEQ ID NO: 68) GN123 (SEQ ID NO: 173) and GN121 (SEQ ID NO: 175). In certain embodiments, the lysin variant has at least 80%, at least 85%, at least 90%, at least 95%, at least 98%, or at least 99% sequence identity to a reference lysin polypeptide having the amino acid sequence selected from the group consisting of SEQ ID NO: 203, 74, 78, 124, 84, 22, 96, 26, 56, 118, 58, 60, 64, 66, 28, 68, 173 and 175.

For example, the GN37 lysin (SEQ ID NO: 84) can be modified to increase the pI by introducing the amino acid substitution, R79H, to generate the GN217 lysin (SEQ ID NO: 8). In this embodiment, the potency of the GN217 lysin (SEQ ID NO: 8) is increased in both the presence and absence of human serum in comparison to that of the reference lysin, GN37 (SEQ ID NO: 84), as described in the examples.

Other examples of suitable pI modifying mutations include introducing an amino acid substitution such as K218D, K228D, R85H and/or K22D into a reference lysin, such as GN316 (SEQ ID NO: 22), to generate e.g., the GN394 lysin (SEQ ID NO: 48), the GN396 lysin (SEQ ID NO: 50), the GN408 lysin (SEQ ID NO: 52) and the GN418 lysin (SEQ ID NO: 54), respectively. In some embodiments, the foregoing pI modifying mutations improve the antibacterial activity of the lysin in the absence and/or presence of human serum as exemplified herein.

In some embodiments, the lysin variants of the present disclosure are typically designed to retain an α-helix domain, the presence or absence of which can be readily determined using various software programs, such as Jpred4 (compio.dundee.ac.uk/jpred), Helical Wheel (hael.net/helical.htm), HeliQuest (zhanglab.ccmb.med.umich.edu/I-TASSER/) and PEP-FOLD 3 (bioserv.rpbs.univ-paris-diderot.fr/services/PEP-FOLD3).

In some embodiments, the α-helix domain is located at the C terminus of a lysin. In other embodiments, the α-helix domain is located at the N-terminus of a lysin. More typically, the α-helix domain is located at the C terminus. The α-helix domain of the lysins of the present disclosure varies in size between about 20 and 40 amino acids, more typically between about 15 and 33 amino acid residues. For example, the GN14 α-helix domain, which is located at the N terminus, contains 15 amino acids (residues 66 to 80 of SEQ ID NO: 124). The GN37 α-helix domain, which is located at the C terminus, contains 14 amino acids (residues 113 to 126 of SEQ ID NO: 84). The GN4 α-helix domain, which is also located at the C terminus, contains 25 amino acids (residues 116 to 140 of SEQ ID NO: 74).

In some embodiments, the variant lysins, active fragments thereof or derivatives thereof disclosed herein are modified to include a purification tag, e.g. GSHHHHHHG (SEQ ID NO: 100). The purification tag may be inserted anywhere within the lysin, typically between the first and second amino acids. For example, the purification tag may be inserted between the first methionine and first alanine at the N terminus of the GN316 lysin (SEQ ID NO: 22) to obtain a variant GN316 lysin (SEQ ID NO: 24) without adversely affecting the activity. In other embodiments, the purification tag may be inserted between the first methionine and the first glycine at the N terminus of the GN156 lysin (SEQ ID NO: 126) to obtain the variant GN486 (SEQ ID NO: 66).

Lysin variants may be formed by any method known in the art and as described in WO WO 2017/049233, which is herein incorporated by reference in its entirety, e.g., by modifying any of the lysins, active fragments thereof and derivatives described herein through site-directed mutagenesis or via mutations in hosts that produce the present lysins which retain one or more of the biological functions as described herein. The present lysin variants may be truncated, chimeric, shuffled or “natural,” and may be in combination as described, for example, in U.S. Pat. No. 5,604,109, which is incorporated herein in its entirety by reference.

For example, one of skill in the art can reasonably make and test substitutions or replacements to, e.g., the α-helix domain or regions outside of the α-helix domain. Sequence comparisons to the Genbank database can be made with e.g., a full amino acid sequence as described herein, for instance, to identify amino acids for substitution.

Mutations can be made in the amino acid sequences, or in the nucleic acid sequences encoding the polypeptides and lysins, active fragments or derivatives, such that a particular codon is changed to a codon which codes for a different amino acid, an amino acid is substituted for another amino acid, or one or more amino acids are deleted.

Such a mutation is generally made by making the fewest nucleotide changes possible. A substitution mutation of this sort can be made to change an amino acid in the resulting protein in a non-conservative manner (for example, by changing the codon from an amino acid belonging to a grouping of amino acids having a particular size or characteristic to an amino acid belonging to another grouping) or in a conservative manner (for example, by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present disclosure should be considered to include sequences containing conservative changes which do not significantly alter the activity or binding characteristics of the resulting protein. Thus, one of skill in the art, based on a review of the sequence of lysins provided herein and on their knowledge and the public information available for other lysin polypeptides, can make amino acid changes or substitutions in the lysin polypeptide sequence. Amino acid changes can be made to replace or substitute one or more, one or a few, one or several, one to five, one to ten, or such other number of amino acids in the sequence of the lysin(s) provided herein to generate mutants or variants thereof. Such mutants or variants thereof may be predicted for function or tested for function or capability for anti-bacterial activity as described herein against, e.g., P. aeruginosa, and/or for having comparable activity to the lysin(s) as described and particularly provided herein. Thus, changes made to the sequence of lysin, and mutants or variants described herein can be tested using the assays and methods known in the art and described herein. One of skill in the art, on the basis of the domain structure of the lysin(s) hereof can predict one or more, one or several amino acids suitable for substitution or replacement and/or one or more amino acids which are not suitable for substitution or replacement, including reasonable conservative or non-conservative substitutions.

In some embodiments, the present isolated polypeptides comprise active fragments of lysins or derivatives. The term “active fragment” refers to a portion of a full-length lysin, which retains one or more biological activities of the reference lysin. Thus, as used herein, an active fragment of a lysin or variant lysin inhibits the growth, or reduces the population, or kills P. aeruginosa and optionally at least one species of Gram-negative bacteria as described herein in the absence or presence of, or in both the absence and presence of, human serum or in the presence of pulmonary surfactant. Suitable active fragments of lysins include, but are not limited, to those described in WO2017/049233, which is herein incorporated by reference in its entirety. The active lysin fragments typically retain an α-helix domain. Examples of active lysin fragments include those of the GN4 lysin (SEQ ID NO: 74) set forth in SEQ ID NOS: 127-129.

In some embodiments, the lysin, variant lysin, active fragment thereof or derivative included in the present isolated polypeptides is selected from the group consisting of GN217 (SEQ ID NO: 8), GN316 variant (SEQ ID NO: 24) GN316 (SEQ ID NO: 22), GN329 (SEQ ID NO: 26), GN333 (SEQ ID NO: 28), GN394 (SEQ ID NO: 48), GN396 (SEQ ID NO: 50), GN408 (SEQ ID NO: 52), (SEQ ID NO: 54), GN424 (SEQ ID NO: 56), GN425 (SEQ ID NO: 58), GN428 (SEQ ID NO: 60), GN431 (SEQ ID NO: 64), GN486 (SEQ ID NO: 66), GN485 (SEQ ID NO: 68), Lysin PaP2_gp17 (SEQ ID NO: 96) GN123 (SEQ ID NO: 173) and GN121 (SEQ ID NO: 175) or an active fragment thereof, wherein the lysin or active fragment thereof inhibits the growth, or reduces the population, or kills P. aeruginosa and optionally at least one other species of Gram-negative bacteria as described herein in the absence or presence of, or in both the absence and presence of, human serum or in the presence of pulmonary surfactant. In some embodiments, the lysin or active fragment thereof contains at least one amino acid substitution, deletion, or insertion relative to at least one of SEQ ID NOS: 8, 24, 22, 26, 28, 48, 50, 52, 54, 56, 58, 60, 64, 66, 68, 96, 173 or 175. In certain embodiments, the at least one amino acid substitution is a conservative amino acid substitution.

In some embodiments, the lysin of the disclosure is selected from the group consisting of GN329 (SEQ ID NO: 26), GN333 (SEQ ID NO: 28), GN424 (SEQ ID NO: 56), GN425 (SEQ ID NO: 58), GN428 (SEQ ID NO: 60), GN431 (SEQ ID NO: 64), GN485 (SEQ ID NO: 68) and Lysin PaP2_gp17 (SEQ ID NO: 96) or an active fragment thereof, wherein the lysin or active fragment thereof inhibits the growth, or reduces the population, or kills P. aeruginosa and optionally at least one other species of Gram-negative bacteria as described herein in the absence or presence of, or in both the absence and presence of, human serum or in the presence of pulmonary surfactant. In some embodiments, the lysin, derivative or active fragment thereof contains at least one substitution, deletion, or insertion modification relative to SEQ ID NOS: 26, 28, 56, 58, 60, 64, 68 or 96. In certain embodiments, the at least one amino acid substitution is a conservative amino acid substitution.

In some embodiments, the isolated polypeptide sequence comprises a lysin selected from the group consisting of GN217 lysin (SEQ ID NO: 8), GN394 lysin (SEQ ID NO: 48), GN396 lysin (SEQ ID NO: 50), GN408 lysin (SEQ ID NO: 52), GN418 lysin (SEQ ID NO: 54) and GN486 (SEQ ID NO: 66) or an active fragment thereof, wherein the lysin or active fragment thereof inhibits the growth, or reduces the population, or kills P. aeruginosa and optionally at least one other species of Gram-negative bacteria as described herein in the absence or presence of, or in both the absence and presence of, human serum or in the presence of pulmonary surfactant. In some embodiments, the lysin or active fragment thereof contains at least one substitution, deletion, or insertion modification relative to SEQ ID NOS: 8, 48, 50, 52, 54, or 66. In certain embodiments, the at least one amino acid substitution is a conservative amino acid substitution.

Anti-Microbial Peptides

In some embodiments, the polypeptides of the present disclosure comprise lysin-Anti-Microbial Peptide (AMP) polypeptide constructs. The lysin-AMP polypeptide constructs comprise an isolated polypeptide comprising a lysin, variant lysin, active fragment thereof or derivative as described herein and an antimicrobial peptide or fragment thereof. The term “antimicrobial peptide” (AMP) as used herein refers to a member of a wide range of short (generally 3 to 50 amino acid residues in length) gene-encoded peptides, typically antibiotics, that can be found in virtually every organism. The term encompasses helical peptides, β-sheet peptides and those that display largely disordered random coil structures. AMPs include defensins, cathelicidins, sushi peptides, cationic peptides, polycationic peptides, amphipathic peptides. hydrophobic peptides and/or AMP-like peptides, e.g., amurin peptides as described herein. Fragments of AMPs, AMP variants and derivatives of AMPs are also encompassed by this term.

The term “AMP activity” as used herein encompasses the ability of an AMP or fragment thereof to kill bacteria, reduce the population of bacteria or inhibit bacterial growth e.g., by penetrating the outer membrane of a Gram-negative bacteria in the presence and/or absence of human serum. Typically, translocation of the AMPs is driven by a primary electrostatic interaction with the lipopolysaccharide portion of the outer membrane followed by cation displacement, membrane disorganization and transient openings, and in some cases, internalization of the AMP.

AMP activity also encompasses the ability of an AMP or fragment thereof to reduce the minimum inhibitory concentration (MIC) of an antibiotic in the presence and/or absence of human serum. Suitable methods for assessing the ability of the present AMPs and fragments thereof to penetrate the outer membrane of Gram-negative bacteria and determining a reduction in the MIC of an antibiotic in the presence and absence of serum are known in the art and include those methods described above for the present lysins, derivatives and active fragments thereof.

In some embodiments, the present AMPs are variant AMPs having at least 80%, such as at least 85%, such as at least 90%, such as at least 95%, such as at least 98% or such as at least 99% sequence identity with any of the AMPs described herein, wherein the variant AMP thereof retains an AMP activity.

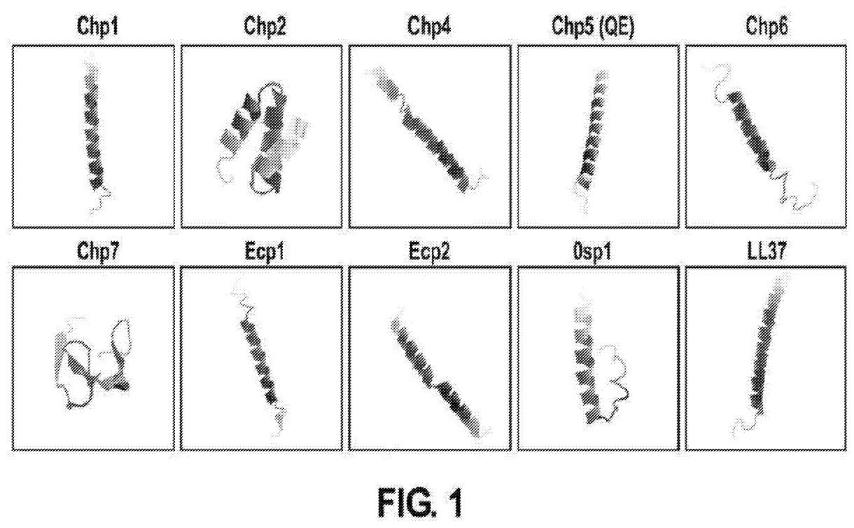

In some embodiments, the present AMPs comprise a helical domain, such as an α-helical domain. In some embodiments, the α-helical domain spans most of the molecule. See, for example, Chp1 and Chp4 of FIG. 1. In other embodiments, the α-helix domain is either interrupted (e.g., Chp2) or truncated (e.g., Chp6 and Osp1). The α-helix domain of the present AMPs, such as the Chps, described herein vary in size from between about 3 to 32 amino acids, more typically between about 10 and 25 amino acid residues. Generally, the helical domains are required for activity and typically must be retained when fused to a C- or N-terminus of a lysin.

Typically, helical peptides display amphipathic characteristics and contain a substantial proportion (e.g. 50%) of hydrophobic residues, frequently appearing in repeated patterns. Upon formation of an α-helical structure, the hydrophilic residues typically end up on the same side of the helix, thereby resulting in a conformation-dependent amphiphilicity. Frequently, these peptides are unstructured in an aqueous environment, but adopt a helical conformation upon encountering lipid membranes. Peptides belonging to this group typically display an overall positive charge ranging from +2 to +11 and usually kill microbes, such as Gram-negative bacteria, by creating membrane defects, leading to a loss of gradients in electrolytes, signal substances and other factors.

In some embodiments, the present AMPs are “AMP-like” peptides including phage lytic agents referred to herein as Chlamydia phage (Chp) peptides or amurin peptides. The amurin peptides of the present disclosure are distinguishable from amurins. As is known in the art, amurins, which are obtained from ssDNA or ssRNA phages (Microviridae and Leviviridae, respectively), are integral membrane proteins with a putative domain structure including an internal LS dipeptide immediately preceded by a stretch of 10-17 hydrophobic residues. Examples of amurins include the protein E amurin from phage <pX174 (Family Microviridae, genus Microvints), which is a 91 amino acid membrane protein that causes lysis by inhibiting the bacterial translocase Mra Y, an essential membrane-embedded enzyme that catalyzes the formation of the murein precursor, Lipid I; the A2 capsid protein of phage Q˜ (Family Leviviridae, genus Allolevivirus), which is a 420-amino acid structural protein that causes lysis by interfering with MurA activity and dysregulating the process of peptidoglycan biosynthesis; the protein L amurin of phage MS2 (Family Levivirdae, genus Levivirus), which is a 75 amino acid integral membrane protein that causes lysis using a mechanism that requires the activity of host chaperone DnaJ. Typically, amurins cannot be purified and are not suitable for use as antibacterial therapeutics.

In contrast to amurins, the amurin peptides of the present disclosure are small cationic peptides with predicted α-helical structures similar to those of AMPs obtained from the innate immune systems of a variety of vertebrates (but with amino acid sequences dissimilar to AMPs). Amurin peptides are primarily found in Chlamydiamicroviruses and, to a lesser extent, in other related members of the subfamily Gokushovirinae. The amurin peptides from a variety of Microviridae phages exhibit 30-100% identity to each other and have no homology with other peptides. Unlike the amurins of Microviridae, which have cytoplasmic targets in the cell wall biosynthetic apparatus, and, accordingly, may not be easily accessed by externally applied proteins, the present amurin peptides can be used in purified form to exert bactericidal activity “from without.”

Suitable amurin peptides for use in the present lysin-AMP polypeptide constructs include those described in U.S. Provisional Application No. 62/650,235, which was filed on 29 Mar. 2018, and which is herein incorporated by reference in its entirety. In some embodiments, amurin peptides such as the chlamydia phage (Chp)-derived lytic agents may be used. Such Chp-derived lytic agents include Chp1 (NCBI Reference Sequence: NP_044319.1, SEQ ID NO: 133), Chp2 (NCBI Reference Sequence: NP_0546521.1, SEQ ID NO: 70), CPAR39 (NCBI Reference Sequence: NP_063898.1, SEQ ID NO: 135), Chp3 (NCBI Reference Sequence: YP_022484.1, SEQ ID NO: 137), Chp4 (NCBI Reference Sequence: YP_338243.1, SEQ ID NO: 102), Chp6 (NCBI Reference Sequence: NP_510878.1, SEQ ID NO: 106), Chp7 (NCBI Reference Sequence: CRH73061.1, SEQ ID NO: 139), Chp8 (NCBI Reference Sequence: CRH64983.1, SEQ ID NO: 141), Chp9 (NCBI Reference Sequence: CRH84960.1, SEQ ID NO: 143), Chp10 (NCBI Reference Sequence: CRH73061.1, SEQ ID NO: 145), Chp11 (NCBI Reference Sequence: CRH59954.1, SEQ ID NO: 147) and Chp12 (NCBI Reference Sequence: CRH59965.1, SEQ ID NO: 149).

Additional, suitable Chp family members include Gkh1 (NCBI Reference Sequence: YP_008798245.1, SEQ ID NO: 151), Gkh2 (NCBI Reference Sequence: YP_009160382.1, SEQ ID NO: 90), Unp1 (NCBI Reference Sequence: CDL66944.1, SEQ ID NO: 153), Ecp1 (NCBI Reference Sequence: WP_100756432.1, SEQ ID NO: 155), Ecp2 (NCBI Reference Sequence: OAC1404.1, SEQ ID NO: 104), Tma1 (NCBI Reference Sequence: SHG47122.1, SEQ ID NO: 157), Osp1 (NCBI Reference Sequence: SFP13761.1, SEQ ID NO: 108), Unp2 (NCBI Reference Sequence: CDL65918.1, SEQ ID NO: 159), Unp3 (NCBI Reference Sequence: CDL65808.1, SEQ ID NO: 161), Gkh3 (NCBI Reference Sequence: AGT39941.1, SEQ ID NO: 163), Unp5 (NCBI Reference Sequence: AGT39924.1, SEQ ID NO: 165), Unp6 (NCBI Reference Sequence: AGT39915.1, SEQ ID NO: 167), Spi1 (NCBI Reference Sequence: NP_598337.1, SEQ ID NO: 169) and Spi2 (NCBI Reference Sequence: NP_598336.1, SEQ ID NO: 171), Ecp3 (NCBI Reference Sequence: WP_105269219.1, SEQ ID NO: 177), Ecp4 (NCBI Reference Sequence: WP_105466506.1, SEQ ID NO: 179), ALCES1 (NCBI Reference Sequence: AXB22573.1, SEQ ID NO: 181), AVQ206 (NCBI Reference Sequence: AVQ10236.1, SEQ ID NO: 183), AVQ244 (NCBI Reference Sequence: AVQ10244.1, SEQ ID NO: 185), CDL907 (NCBI Reference Sequence: CDL65907.1, SEQ ID NO: 187), AGT915 (NCBI Reference Sequence: AGT39915.1, SEQ ID NO: 189), HH3930 (NCBI Reference Sequence: CCH66548.1, SEQ ID NO: 191), Fen7875 (NCBI Reference Sequence: YP_009160399.1, SEQ ID NO: 193), SBR77 (NCBI Reference Sequence: AOT25441, SEQ ID NO: 195), Bdp1 NCBI Reference Sequence: NP_073546.1, SEQ ID NO: 197), LVP1 (NCBI Reference Sequence: NP_042306.1, SEQ ID NO: 199) and Lvp2 (NCBI Reference Sequence: NP_085469.1, SEQ ID NO: 201).

More typically, the AMPs are selected from one or more of the following amurin peptides, Chp2 (SEQ ID NO: 70), Gkh2 (SEQ ID NO: 90), Chp4 (SEQ ID NO: 102), Ecp2 (SEQ ID NO: 104), Chp6 (SEQ ID NO: 106) and Osp1 (SEQ ID NO: 108).

In some embodiments, the amurin peptides are modified to produce variant amurin peptides. As described herein, amurin peptides typically comprise a helical domain such as an α-helical domain. Typically, the variant amurin peptides retain the α-helical domain. The retention of the α-helical domain in any variant amurin peptide is typically accurately identified using various software programs, such as Jpred4 (compio.dundee.ac.uk/jpred), Helical Wheel (hael.net/helical.htm), HeliQuest (zhanglab.ccmb.med.umich.edu/I-TASSER/) and PEP-FOLD 3 (bioserv.rpbs.univ-paris-diderot.fr/services/PEP-FOLDS). In some embodiments, the amurin peptide variants are modified by converting (=) charged residues, such as arginine and lysine, within the amurin peptide to a “D” amino acid form. The utility of conversions to the D form is described in the literature, e.g., Manabe et al., Sci. Rep., 2017, pages 1-10, which is herein incorporated by reference in its entirety. Variant AMPs may be prepared according to any method known in the art including as described herein above for the lysins, variants, active fragments thereof and derivatives.

In some embodiments, the AMPs for use in the lysin-AMP polypeptide constructs of the present disclosure include a fragment of a larger AMP that retains antibacterial activity. For example, in certain embodiments, the AMP portion of the lysin-AMP polypeptide construct may include a fragment of porcine myeloid antimicrobial peptide-36 (“PMAP-36”, SEQ ID NO: 204) that retains antibacterial activity. PMAP-36 is a cathelicidi n-related AMP deduced from porcine myeloid cDNA with an amphipathic α-helical conformation at the N-terminus. Accordingly, suitable PMAP-36 fragments are typically selected from the N-terminus to obtain fragments retaining antibacterial activity. In some embodiments, the PMAP-36 fragment of the present disclosure includes the hydrophobic amino acid (Trp) at position 23. In other embodiments, the random coil C-terminal is omitted from the PMAP-36 fragment to reduce or eliminate hemolysis that may be caused by PMAP-36. Further features of PMAP-36 fragments are described, for example, in Lyu et al., Scientific Reports, 2016, 6, pages 1-12, which is herein incorporated by reference in its entirety.

Particularly desirable PMAP-36 fragments include RI12 (SEQ ID NO: 88), RI18 (SEQ ID NO: 92) and TI15 (SEQ ID NO: 94). Other suitable AMP fragments include those from Esculentin (NCBI Reference Sequence: P40843.1), such as the fragment set forth in SEQ ID NO: 80 and anti-lipopolysaccharide factor isoform 2 (NCBI Reference Sequence: AFU61125.1), such as the fragment set forth in SEQ ID NO: 76.

In some embodiments, the AMPs of the present disclosure include synthetic peptides. In some embodiments, the synthetic peptide reduces the minimum inhibitory concentration (MIC) of an antibiotic, which prevents visible growth of bacterium, but does not itself exhibit antibacterial activity. A particularly desirable synthetic peptide for use with the lysin-AMP polypeptide constructs of the present disclosure includes the FIRL peptidomimetic (SEQ ID NO: 114). Without being limited by theory, FIRL (SEQ ID NO: 114), which is related to a sequence of a protein involved in outer membrane protein biogenesis, BamD, appears to increase the permeability of the outer membrane to antibiotics. Further information regarding the proposed mechanism is found, for example, in Mori et al., Journal of Antimicrobial Chemotherapy, 2012, 67: 2173-2181, which is herein incorporated by reference in its entirety.

Other synthetic peptides useful for sensitizing gram-negative bacteria to antibiotics, which may be incorporated into the lysin-AMP polypeptide construct of the present disclosure includes the cationic peptide KFFKFFKFFK (SEQ ID NO: 120) described in Vaara and Porro, Antimicrobial agents and Chemotherapy, 1996, 1801-1805, which is herein incorporated by reference in its entirety.

In some embodiments, the synthetic peptides are resistant to salts and serum inactivation as described, for example, in Monhanram et al., Biopolymers, 2016, 106: 345-346, which is herein incorporated by reference in its entirety. Particularly desirable salt and serum-resistant synthetic peptides include RR12Whydro (SEQ ID NO: 110) and RI18 peptide derivative (SEQ ID NO: 131).

Structure Stabilizing Components

In some embodiments, the lysin-AMP polypeptide constructs of the present disclosure further include at least one structure stabilizing component to maintain at least a portion of the structure of the first and/or second component in the construct, e.g., the lysin and/or AMP, substantially the same as in the unconjugated lysin and/or AMP. In some embodiments, the stabilizing structure is a linker. Typically, the at least one structure stabilizing component, such as a linker enables the lysin and AMP to substantially preserve the three-dimensional structure of the first and/or second protein moieties, such that at least one biological activity of the lysin and/or AMP is retained.

Suitable linkers for connecting two polypeptides are known in the art. In certain embodiments, the linker is a peptide, such as a peptide comprising glycine and serine residues. Specific suitable linkers include, but are not limited to, a TAGGTAGG linker (SEQ ID NO: 72), an IGEM linker GGSGSGSGSGSP (BBa_K1485002) (SEQ ID NO: 82). GGGSGGGGSGGGS (BBA_K1486037, (SEQ ID NO: 86), or a linker as described in Briers et al., mBio, 2014, 5:e01379-14, which is herein incorporated by reference in its entirety, i.e., AGAGAGAGAGAGAGAGAS (SEQ ID NO: 122).

In some embodiments, the structure stabilizing component is a peptide moiety, e.g., an RPP or PP moiety. Such peptide moieties may be included in the present lysin-AMP polypeptide constructs to assist in maintaining the structure of the lysin and/or AMP protein moieties. For example, the RPP or PP amino acid may be inserted at the C terminus or N terminus of a linker, e.g. at the N terminus of the BBA_K1486037 linker (RPPGGGSGGGGSGGGS residues 126 to 141 of SEQ ID NO: 12), at the N terminus of the BBA_K1486037 linker (PPGGGSGGGGSGGGS, residues 144-158 of SEQ ID NO: 16), at the N terminus of the TAGGTAGG linker (SEQ ID NO: 72), such as depicted in residues 137-144 of SEQ ID NO: 18) or at the C terminus of the BBA_K1486037 linker (GGGSGGGGSGGGSPP, residues 135-149 of SEQ ID NO: 20).

In other embodiments, the peptides MIDR (SEQ ID NO: 112) and/or NPTH (SEQ ID NO: 116) are included in the construct to assist in maintaining the structure of the lysin and/or AMP protein moieties. For example, in some embodiments an AMP structure, such as FIRL (SEQ ID NO: 114), is maintained by the addition of MIDR (SEQ ID NO: 112) and/or NPTH (SEQ ID NO: 116) such as depicted at residues 1-12 of SEQ ID NO: 46 (MIDRFIRLNPTH) and residues 1-26 of SEQ ID NO: 44.

Examples of Lysin-AMP Polypeptide Constructs

In some embodiments, the lysin-AMP construct comprises: (a) a first component comprising (i) at least one lysin selected from the group consisting of GN76 (SEQ ID NO: 203), GN4 (SEQ ID NO: 74), GN146 (SEQ ID NO: 78), GN14 (SEQ ID NO: 124), GN37 (SEQ ID NO: 84) optionally with a single pI-increasing mutation, GN316 (SEQ ID NO: 22) optionally with a single point mutation, lysin Pap2_gp17 (SEQ ID NO: 96), GN329 (SEQ ID NO: 26), GN424 (SEQ ID NO: 56), GN202 (SEQ ID NO: 118), GN425 (SEQ ID NO: 58), GN428 (SEQ ID NO: 60), GN431 (SEQ ID NO: 64), GN486 (SEQ ID NO: 66), GN333 (SEQ ID NO: 28), GN485 (SEQ ID NO: 68), GN123 (SEQ ID NO: 173) and GN121 (SEQ ID NO: 175) or (ii) a polypeptide having lysin activity and having at least 80%, such as at least such as at least 85%, such as at least 90%, such as at least 95%, such as at least 98%, or such as at least 99% sequence identity with the polypeptide sequence of any of SEQ ID NOs: 203, 74, 78, 124, 84, 22, 96, 26, 56, 118, 58, 60, 64, 66, 28, 68, 173 or 175; or (iii) an active fragment of the lysin, said fragment including single point mutations and/or single pI increasing mutations if any; (b) a second component comprising (i) at least one antimicrobial peptide (AMP) selected from the group consisting of Chp1 (SEQ ID NO: 133), Chp2 (SEQ ID NO: 70), CPAR39 (SEQ ID NO: 135), Chp3 (SEQ ID NO: 137), Chp4 (SEQ ID NO: 102), Chp6 (SEQ ID NO: 106), Chp7 (SEQ ID NO: 139), Chp8 (SEQ ID NO: 141), Chp9 (SEQ ID NO: 143), Chp10 (SEQ ID NO: 145), Chp11 (SEQ ID NO: 147), Chp12 (SEQ ID NO: 149), Gkh1 (SEQ ID NO: 151), Gkh2 (SEQ ID NO: 90), Unp1 (SEQ ID NO: 153), Ecp1 (SEQ ID NO: 155), Ecp2 (SEQ ID NO: 104), Tma1 (SEQ ID NO: 157), Osp1 (SEQ ID NO: 108), Unp2 (SEQ ID NO: 159), Unp3 (SEQ ID NO: 161), Gkh3 (SEQ ID NO: 163), Unp5 (SEQ ID NO: 165), Unp6 (SEQ ID NO: 167), Spi1 (SEQ ID NO: 169), Spi2 (SEQ ID NO: 171), Ecp3 (SEQ ID NO: 177), Ecp4 (SEQ ID NO: 179), ALCES1 (SEQ ID NO: 181), AVQ206 (SEQ ID NO: 183), AVQ244 (SEQ ID NO: 185), CDL907 (SEQ ID NO: 187), AGT915 (SEQ ID NO: 189), HH3930 (SEQ ID NO: 191), Fen7875 (SEQ ID NO: 193), SBR77 (SEQ ID NO: 195), Bdp1 (SEQ ID NO: 197), LVP1 (SEQ ID NO: 199), Lvp2 (SEQ ID NO: 201), an esculentin fragment (SEQ ID NO: 80), RI12 (SEQ ID NO: 88), TI15 (SEQ ID NO: 94), RI18 (SEQ ID NO: 92), FIRL (SEQ ID NO: 114), a fragment of LPS binding protein (SEQ ID NO: 76), RR12whydro (SEQ ID NO: 110), RI18 peptide derivative (SEQ ID NO: 131) and cationic peptide (SEQ ID NO: 120) or (ii) a polypeptide having AMP activity, wherein the polypeptide is at least 80% identical to at least one of SEQ ID NOS: 133, 70, 135, 137, 102, 106, 139, 141, 143, 145, 147, 149, 151, 90, 153, 155, 104, 157, 108, 159, 161, 163, 165, 167, 169, 171, 177, 179, 181, 183, 185, 187, 189, 191, 193, 195, 197, 199, 201, 80, 88, 94, 92, 114, 76, 110, 131 and 120.

Typically, any of the AMP variants sharing at least 80% identity or more with the disclosed AMPs or fragments thereof retain its alpha-helical structure and any residues associated with activity. For example, as noted above, fragments of PMAP-36 (SEQ ID NO: 204) typically retain the hydrophobic amino acid (Trp) at position 23.

In some embodiments, GN37 (SEQ ID NO: 84) comprises a single pI-increasing mutation, wherein the GN37 (SEQ ID NO: 84) with the single pI-increasing mutation is GN217 (SEQ ID NO: 8). In some embodiments, GN316 (SEQ ID NO: 22) comprises a single point mutation, wherein the GN37 (SEQ ID NO: 84) with the single point mutation is GN396 (SEQ ID NO: 50), GN408 (SEQ ID NO: 52), GN418 (SEQ ID NO: 54) and/or GN394 (SEQ ID NO: 48).

In some embodiments, the construct further comprises at least one structure stabilizing component. In some embodiments, the at least one structure stabilizing component is a peptide linker, such as a peptide comprising glycine and serine residues. In certain embodiments, the peptide linker is selected from the group consisting of TAGGTAGG (SEQ ID NO: 72), IGEM (BBa_K1485002) (SEQ ID NO: 82), PPTAGGTAGG (SEQ ID NO: 98), IGEM +PP (residues 44-58 of SEQ ID NO: 16) and AGAGAGAGAGAGAGAGAS (SEQ ID NO: 122).

In some embodiments, the lysin-AMP polypeptide construct is selected from at least one of GN168 lysin (SEQ ID NO: 2), GN176 lysin (SEQ ID NO: 4), GN178 lysin (SEQ ID NO: 6), GN218 lysin (SEQ ID NO: 10), GN223 lysin (SEQ ID NO: 12), GN239 lysin (SEQ ID NO: 14), GN243 lysin (SEQ ID NO: 16), GN280 lysin (SEQ ID NO: 18), GN281 lysin (SEQ ID NO: 20), GN349 lysin (SEQ ID NO: 30), GN351 lysin (SEQ ID NO: 32), GN352 lysin (SEQ ID NO: 34), GN353 lysin (SEQ ID NO: 36), GN357 lysin (SEQ ID NO: 38), GN359 lysin (SEQ ID NO: 40), GN369 lysin (SEQ ID NO: 42), GN370 lysin (SEQ ID NO: 44), GN371 lysin (SEQ ID NO: 46) or GN 93 lysin (SEQ ID NO: 62) or a polypeptide having lysin activity and having at least 80%, such as at least such as at least 85%, such as at least 90%, such as at least 95%, such as at least 98%, or such as at least 99% sequence identity with the polypeptide sequence of at least one of SEQ ID NOs: 2, 4, 6, 10, 12, 14, 16, 18, 20, 30, 32, 34, 36, 38, 40, 42, 44, 46, or 62.

More particularly, in some embodiments, the lysin-AMP polypeptide construct comprises a Chp2 amurin polypeptide (SEQ ID NO: 70) and a TAGGTAGG linker (SEQ ID NO: 72) introduced N-terminally to the GN4 lysin (SEQ ID NO: 74) to generate the GN168 lysin (SEQ ID NO: 2) or a polypeptide having lysin activity and having at least 80%, such as at least 85%, such as at least 90%, such as at least 95%, such as at least 98%, or such as at least 99% sequence identity to SEQ ID NO: 2.

In some embodiments, the encoded lysin-AMP polypeptide construct comprises a fragment of LPS binding protein (SEQ ID NO: 76) and a TAGGTAGG linker (SEQ ID NO: 72) introduced N-terminally to the GN146 lysin (SEQ ID NO: 78) to generate the GN176 lysin (SEQ ID NO: 4) or a polypeptide having lysin activity and having at least 80%, such as at least 85%, such as at least 90%, such as at least 95%, such as at least 98%, or such as at least 99% sequence identity to SEQ ID NO: 4.

In some embodiments, the lysin-AMP polypeptide construct comprises an Esculentin fragment (SEQ ID NO: 80) and an IGEM linker (SEQ ID NO: 82) introduced N-terminally to the GN146 lysin (SEQ ID NO: 78) to generate the GN178 lysin (SEQ ID NO: 6) or a polypeptide having at least 80%, such as at least 85%, such as at least 90%, such as at least 95%, such as at least 98%, or such as at least 99% sequence identity to SEQ ID NO: 6.

In some embodiments, the encoded lysin-AMP polypeptide construct comprises an IGEM linker (SEQ ID NO: 86) and an RI12 antimicrobial peptide (SEQ ID NO: 88) introduced C-terminally to the GN37 lysin (SEQ ID NO: 84) to generate the GN218 lysin (SEQ ID NO: 10) or a polypeptide having lysin activity and having at least 80%, such as at least 85%, such as at least 90%, such as at least 95%, such as at least 98%, or such as at least 99% sequence identity to SEQ ID NO: 10.

In some embodiments, the lysin-AMP polypeptide construct comprises an RPP moiety, an IGEM linker (SEQ ID NO: 86), and the antimicrobial amurin peptide Gkh2 (SEQ ID NO: 90) introduced C-terminally to the GN37 lysin (SEQ ID NO: 84) to generate the GN223 lysin (SEQ ID NO: 12) or a polypeptide having lysin activity and having at least 80%, such as at least 85%, such as at least 90%, such as at least 95%, such as at least 98% or such as at least 99% sequence identity to SEQ ID NO: 12.

In some embodiments, the lysin-AMP polypeptide construct comprises an IGEM linker (SEQ ID NO: 86) and an RI18 peptide (SEQ ID NO: 92) introduced C-terminally to the GN37 lysin (SEQ ID NO: 84) to generate the GN239 lysin (SEQ ID NO: 14) or a polypeptide having lysin activity and having at least 80%, such as at least 85%, such as at least 90%, such as at least 95%, such as at least 98%, or such as at least 99% sequence identity to SEQ ID NO: 14.

In some embodiments, the lysin-AMP polypeptide construct comprises a PP amino acid moiety, an IGEM linker (SEQ ID NO: 86) and a TI15 peptide (SEQ ID NO: 94), introduced C-terminally to the GN37 lysin (SEQ ID NO: 84) to generate the GN243 lysin (SEQ ID NO: 16) or a polypeptide having lysin activity and having at least 80%, such as at least 85%, such as at least 90%, such as at least 95%, such as at least 98%, or such as at least 99% sequence identity to SEQ ID NO: 16.

In some embodiments, the lysin-AMP polypeptide construct comprises an RI18 antimicrobial peptide (SEQ ID NO: 92), a linker having the amino acid sequence PPTAGGTAGG (SEQ ID NO: 98), and a TI15 antimicrobial peptide (SEQ ID NO: 94) introduced C terminally to a Lysin PaP2_gp17 (SEQ ID NO: 96) to generate GN280 lysin (SEQ ID NO: 18) or a polypeptide having lysin activity and having at least 80%, such as at least 85%, such as at least 90%, such as at least 95%, such as at least 98%, or such as at least 99% sequence identity to SEQ ID NO: 18.

In some embodiments, the lysin-AMP polypeptide construct comprises an RI18 peptide (SEQ ID NO: 92), an IGEM linker (SEQ ID NO: 86), a PP amino acid moiety (added to maintain structure of the lysin and/or the AMP), and a TI15 peptide (SEQ ID NO: 94) introduced C terminally to a Lysin PaP2_gp17 (SEQ ID NO: 96) to generate GN281 lysin (SEQ ID NO: 20) or a polypeptide having lysin activity and having at least 80%, such as at least 85%, such as at least 90%, such as at least 95%, such as at least 98%, or such as at least 99% sequence identity to SEQ ID NO: 20.

In some embodiments, the lysin-AMP polypeptide construct comprises a linker having the amino acid sequence TAGGTAGG (SEQ ID NO: 72), and an amurin peptide Chp4 (SEQ ID NO: 102) introduced C-terminally to the GN316 lysin (SEQ ID NO: 22) to generate the GN349 lysin (SEQ ID NO: 30) or a polypeptide having lysin activity and having at least 80%, such as at least 85%, such as at least 90%, such as at least 95%, such as at least 98%, or such as at least 99% sequence identity to SEQ ID NO: 30.

In some embodiments, the lysin-AMP polypeptide construct comprises a linker having the amino acid sequence TAGGTAGG (SEQ ID NO: 72), and an amurin peptide Ecp2 (SEQ ID NO: 104), introduced C-terminally to the GN316 lysin (SEQ ID NO: 22) to generate the GN351 lysin (SEQ ID NO: 32) or a polypeptide having lysin activity and having at least 80%, such as at least 85%, such as at least 90%, such as at least 95%, such as at least 98%, or such as at least 99% sequence identity to SEQ ID NO: 32.

In some embodiments, the lysin-AMP polypeptide construct comprises a linker having the amino acid sequence TAGGTAGG (SEQ ID NO: 72), and an amurin peptide Chp7 (SEQ ID NO: 139) introduced C-terminally to the GN316 lysin (SEQ ID NO: 22) to generate the GN352 lysin (SEQ ID NO: 34) or a polypeptide having lysin activity and having at least 80%, such as at least 85%, such as at least 90%, such as at least 95%, such as at least 98%, or such as at least 99% sequence identity to SEQ ID NO: 34.

In some embodiments, the lysin-AMP polypeptide construct comprises a linker having the amino acid sequence TAGGTAGG (SEQ ID NO: 72) and an amurin peptide Osp1 (SEQ ID NO: 108), introduced C-terminally to the GN316 lysin (SEQ ID NO: 22) to generate the GN353 lysin (SEQ ID NO: 36) or a polypeptide having lysin activity and having at least 80%, such as at least 85%, such as at least 90%, such as at least 95%, such as at least 98%, or such as at least 99% sequence identity to SEQ ID NO: 36.

In some embodiments, the lysin-AMP polypeptide construct comprises a linker having the amino acid sequence TAGGTAGG (SEQ ID NO: 72), and a RR12Whydro (SEQ ID NO: 110) introduced C-terminally to the GN316 lysin (SEQ ID NO: 22) to generate the GN357 lysin (SEQ ID NO: 38) or a polypeptide having lysin activity and having at least 80%, such as at least 85%, such as at least 90%, such as at least 95%, such as at least 98%, or such as at least 99% sequence identity to SEQ ID NO: 38.

In some embodiments, the lysin-AMP polypeptide construct comprises a linker having the amino acid sequence TAGGTAGG (SEQ ID NO: 72) and a TI15 peptide derivative of PMAP-36 (SEQ ID NO: 94), introduced C-terminally to the GN316 lysin (SEQ ID NO: 22) to generate the GN359 lysin (SEQ ID NO: 40) or a polypeptide having lysin activity and having at least 80%, such as at least 85%, such as at least 90%, such as at least 95%, such as at least 98%, or such as at least 99% sequence identity to SEQ ID NO: 40.

In some embodiments, the lysin-AMP polypeptide construct comprises RR18 (SEQ ID NO: 92), introduced C-terminally to the GN316 lysin (SEQ ID NO: 22) to generate the GN369 lysin (SEQ ID NO: 42) or a polypeptide having lysin activity and having at least 80%, such as at least 85%, such as at least 90%, such as at least 95%, such as at least 98%, or such as at least 99% sequence identity to SEQ ID NO: 42.

In some embodiments, the lysin-AMP polypeptide construct comprises a MDR moiety (SEQ ID NO: 112), a FIRL moiety (SEQ ID NO:114) and an NPTH moiety (SEQ ID NO: 116) introduced N-terminally to the GN202 lysin (SEQ ID NO: 118) to generate the GN370 lysin (SEQ ID NO: 44) or a polypeptide having lysin activity and having at least 80%, such as at least 85%, such as at least 90%, such as at least 95%, such as at least 98%, or such as at least 99% sequence identity to SEQ ID NO: 44.

In some embodiments, the lysin-AMP polypeptide construct comprises a MDR moiety (SEQ ID NO: 112), FIRL (SEQ ID NO: 114) and an NPTH moiety (SEQ ID NO: 116) introduced C-terminally to the GN146 lysin (SEQ ID NO: 78) to generate the GN371 lysin (SEQ ID NO: 46) or a polypeptide having lysin activity and having at least 80%, such as at least 85%, such as at least 90%, such as at least 95%, such as at least 98%, or such as at least 99% sequence identity to SEQ ID NO: 46.

In some embodiments, the lysin-AMP polypeptide construct comprises a cationic peptide (SEQ ID NO: 120) and a linker domain (SEQ ID NO: 122) introduced N-terminally to the GN14 lysin (SEQ ID NO: 124) to generate a GN93 lysin (SEQ ID NO: 62) or a polypeptide having lysin activity and having at least 80%, such as at least 85%, such as at least 90%, such as at least 95%, such as at least 98%, or such as at least 99% sequence identity to SEQ ID NO: 62.

Table 1, below, depicts specific examples of the lysins and lysin-AMP constructs described herein. The AMP portion of the construct is double-underlined for GN168 (SEQ ID NO: 2), GN176 (SEQ ID NO: 4), GN178 (SEQ ID NO: 6), GN370 (SEQ ID NO: 44), GN371 (SEQ ID NO: 46) and GN93 (SEQ ID NO: 62). For all other constructs, double underlines correspond to a lysin. Structure stabilizing components, such as linkers are italicized. The purification tag for GN486 (SEQ ID NO: 66) is italicized and bolded. Single point mutations are bolded.

TABLE 1

| GN # | Polypeptide Sequence |
|---|---|
| GN168 | MRLKMARRRYRLPRRRSRRLFSRTALRMHPRNRLRRIMRGGIRF [symbol] |
| | [symbol] RTSQRGIDLIKSFEGLRLSAYQDSVGVWTIGYGTTRGVTRYMTITVEQ |
| | AERMLSNDIQRFEPELDRLAKVPLNQNQWDALMSFVYNLGAANLASSTLL |
| | DLLNKGDYQGAADQFPHWVNAGGKRLDGLVKRRAAERALFLEPLS |
| | (SEQ ID NO: 2) |
| GN176 | MSFNVTPKFKRWQLYFRGRMW [symbol] RTSQRGIDLIKSFEGLRLSAY |
| | QDSVGVWTIGYGTTRGVTRYMTITVEQAERMLSNDIQRFEPELDRLAKVP |
| | LNQNQWDALMSFVYNLGAANLASSTLLDLLNKGDYQGAADQFPHWVNAGG |
| | KRLDGLVKRRAAERALFLEPLS (SEQ ID NO: 4) |
| GN178 | MPPIFSKLAGKKIKNLLISGLK [symbol] RTSQRGIDLIKSFEGL |
| | RLSAYQDSVGVWTIGYGTTRGVTRYMTITVEQAERMLSNDIQRFEPELDR |
| | LAKVPLNQNQWDALMSFVYNLGAANLASSTLLDLLNKGDYQGAADQFPHW |
| | VNAGGKRLDGLVKRRAAERALFLEPLS (SEQ ID NO: 6) |
| GN217 | MTYTLSKRSLDNLKGVHPDLVAVVHRAIQLTPVDFAVIEGLRSVSRQKEL |
| | VAAGASKTMNSRHLTGHAVDLAAYVNGIHWDWPLYDAIAVAVKAAAKELG |
| | VAIVWGGDWTTFKDGPHFELDRSKYR (SEQ ID NO: 8) |
| GN218 | MTYTLSKRSLDNLKGVHPDLVAVVHRAIQLTPVDFAVIEGLRSVSRQKEL |
| | VAAGASKTMNSRHLTGHAVDLAAYVNGIRWDWPLYDAIAVAVKAAAKELG |
| | VAIVWGGDWTTFKDGPHFELDRSKY [symbol] RLKKIGKVLKWI |
| | (SEQ ID NO: 10) |
| GN223 | MTYTLSKRSLDNLKGVHPDLVAVVHRAIQLTPVDFAVIEGLRSVSRQKEL |
| | VAAGASKTMNSRHLTGHAVDLAAYVNGIRWDWPLYDAIAVAVKAAAKELG |
| | VAIVWGGDWTTFKDGPHFELDRSKYRPP [symbol] SKKASRKSF |
| | TKGAVKVHKKNVPTRVPMRGGIRL (SEQ ID NO: 12) |
| GN239 | MTYTLSKRSLDNLKGVHPDLVAVVHRAIQLTPVDFAVIEGLRSVSRQKEL |
| | VAAGASKTMNSRHLTGHAVDLAAYVNGIRWDWPLYDAIAVAVKAAAKELG |
| | VAIVWGGDWTTFKDGPHFELDRSKY [symbol] RKKTRKRLKKIG |
| | KVLKWI (SEQ ID NO: 14) |
| GN243 | MTYTLSKRSLDNLKGVHPDLVAVVHRAIQLTPVDFAVIEGLRSVSRQKEL |
| | VAAGASKTMNSRHLTGHAVDLAAYVNGIRWDWPLYDAIAVAVKAAAKELG |
| | VAIVWGGDWTTFKDGPHFELDRSKYRKKTRKRLKKIGKVLKWIPP [symbol] |
| | [symbol] TRKRLKKIGKVLKWI (SEQ ID NO: 16) |
| GN280 | MKLSEKRALFTQLLAQLILWAGTQDRVSVALDQVKRTQAEADANAKSGAG |
| | IRNSLHLLGLAGDLILYKDGKYMDKSEDYKFLGDYWKSLHPLCRWGGDFK |
| | SRPDGNHFSLEHEGVQ [symbol] PPTAGGTAGGTRKRLK |
| | KIGKVLKWI (SEQ ID NO: 18) |
| GN281 | MKLSEKRALFTQLLAQLILWAGTQDRVSVALDQVKRTQAEADANAKSGAG |
| | IRNSLHLLGLAGDLILYKDGKYMDKSEDYKFLGDYWKSLHPLCRWGGDFK |
| | SRPDGNHFSLEHEGVQ [symbol] GGGSGGGGSGGGSPPT |
| | RKRLKKIGKVLKWI (SEQ ID NO: 20) |
| GN316 | MAILKIGSKGLEVKNLQTSLNKIGFNLVADGIFGKATDNAVRAVQAGAGL |
| | VVDGIAGPKTMYAIRNAGESHQDHLTEADLIDAARELSVDLASIKAVNQV |
| | ESRGTGFTKSGKIKTLFERHIMYKKLNAKFGQAKANALAQLYPTLVNAKA |
| | GGYTGGDAELERLHGAIAIDKDCAYESASYGLFQIMGFNCVICGYDNAEE |
| | MFNDFLTGERAQLMAFVKFIKADANLWKALKDKNWAEFARRYNGPAYAQN |
| | QYDTKLAAAYKSFS (SEQ ID NO: 22) |
| GN329 | MITDREYQQAAEMLGVDVPAIKAVTKVEAPVGGFQPTGEPTILYERHQMY |
| | RQLQAKGLPTEGHPPDLVNKVAGGYGKYSEQHAKLARAVKIDRDSALESC |
| | SWGMFQIMGYHWKLMGYPTLQAFVNAMYASEGAQMDAFCRFIKAQPTTHA |
| | ALKAHDWAKFARLYNGPGYAKNKYDVKLEKAYAEASG (SEQ ID NO: |
| | 26) |
| GN333 | MALTEQDFQSAADDLGVDVASVKAVTKVESRGSGFLLSGVPKILFERHWM |
| | FKLLKRKLGRDPEINDVCNPKAGGYLGGQAEHERLDKAVKMDRDCALQSA |
| | SWGLFQIMGFHWEALGYASVQAFVNAQYASEGSQLNTFVRFIKTNPAIHK |
| | ALKSKDWAEFARRYNGPDYKKNNYDVKLAEAYQSFK (SEQ ID NO: |
| | 28) |
| GN349 | MAILKIGSKGLEVKNLQTSLNKIGFNLVADGIFGKATDNAVRAVQAGAGL |
| | VVDGIAGPKTMYAIRNAGESHQDHLTEADLIDAARELSVDLASIKAVNQV |
| | ESRGTGFTKSGKIKTLFERHIMYKKLNAKFGQAKANALAQLYPTLVNAKA |
| | GGYTGGDAELERLHGAIAIDKDCAYESASYGLFQIMGFNCVICGYDNAEE |
| | MFNDFLTGERAQLMAFVKFIKADANLWKALKDKNWAEFARRYNGPAYAQN |
| | QYDTKLAAAYKSFS [symbol] ARRYRLSRRRSRRLFSRTALRMHRRNRL |
| | RRIMRGGIRF (SEQ ID NO: 30) |
| GN351 | MAILKIGSKGLEVKNLQTSLNKIGFNLVADGIFGKATDNAVRAVQAGAGL |
| | VVDGIAGPKTMYAIRNAGESHQDHLTEADLIDAARELSVDLASIKAVNQV |
| | ESRGTGFTKSGKIKTLFERHIMYKKLNAKFGQAKANALAQLYPTLVNAKA |
| | GGYTGGDAELERLHGAIAIDKDCAYESASYGLFQIMGFNCVICGYDNAEE |
| | MFNDFLTGERAQLMAFVKFIKADANLWKALKDKNWAEFARRYNGPAYAQN |
| | QYDTKLAAAYKSFS [symbol] RSRRRMSKRSSRRSFRKYAKSHKKNFKA |
| | RSMRGGIRL (SEQ ID NO: 32) |
| GN352 | MAILKIGSKGLEVKNLQTSLNKIGFNLVADGIFGKATDNAVRAVQAGAGL |
| | VVDGIAGPKTMYAIRNAGESHQDHLTEADLIDAARELSVDLASIKAVNQV |
| | ESRGTGFTKSGKIKTLFERHIMYKKLNAKFGQAKANALAQLYPTLVNAKA |
| | GGYTGGDAELERLHGAIAIDKDCAYESASYGLFQIMGFNCVICGYDNAEE |
| | MFNDFLTGERAQLMAFVKFIKADANLWKALKDKNWAEFARRYNGPAYAQN |
| | QYDTKLAAAYKSFS [symbol] KRRKMTRKGSKRLFTATADKTKSINTAP |
| | PPMRGGIRL (SEQ ID NO: 34) |
| GN353 | MAILKIGSKGLEVKNLQTSLNKIGFNLVADGIFGKATDNAVRAVQAGAGL |
| | VVDGIAGPKTMYAIRNAGESHQDHLTEADLIDAARELSVDLASIKAVNQV |
| | ESRGTGFTKSGKIKTLFERHIMYKKLNAKFGQAKANALAQLYPTLVNAKA |
| | GGYTGGDAELERLHGAIAIDKDCAYESASYGLFQIMGFNCVICGYDNAEE |
| | MFNDFLTGERAQLMAFVKFIKADANLWKALKDKNWAEFARRYNGPAYAQN |
| | QYDTKLAAAYKSFS [symbol] RKRMSKRVDKKVFRRTAASAKKINIDPK |
| | IYRGGIRL (SEQ ID NO: 36) |
| GN357 | MAILKIGSKGLEVKNLQTSLNKIGFNLVADGIFGKATDNAVRAVQAGAGL |
| | VVDGIAGPKTMYAIRNAGESHQDHLTEADLIDAARELSVDLASIKAVNQV |
| | ESRGTGFTKSGKIKTLFERHIMYKKLNAKFGQAKANALAQLYPTLVNAKA |
| | GGYTGGDAELERLHGAIAIDKDCAYESASYGLFQIMGFNCVICGYDNAEE |
| | MFNDFLTGERAQLMAFVKFIKADANLWKALKDKNWAEFARRYNGPAYAQN |
| | QYDTKLAAAYKSFS [symbol] RRLIRLWLRLLR (SEQ ID NO: 38) |
| GN359 | MAILKIGSKGLEVKNLQTSLNKIGFNLVADGIFGKATDNAVRAVQAGAGL |
| | VVDGIAGPKTMYAIRNAGESHQDHLTEADLIDAARELSVDLASIKAVNQV |
| | ESRGTGFTKSGKIKTLFERHIMYKKLNAKFGQAKANALAQLYPTLVNAKA |
| | GGYTGGDAELERLHGAIAIDKDCAYESASYGLFQIMGFNCVICGYDNAEE |
| | MFNDFLTGERAQLMAFVKFIKADANLWKALKDKNWAEFARRYNGPAYAQN |
| | QYDTKLAAAYKSFS [symbol] TRKRLKKIGKVLKWI (SEQ ID NO: |
| | 40) |
| GN369 | MAILKIGSKGLEVKNLQTSLNKIGFNLVADGIFGKATDNAVRAVQAGAGL |
| | VVDGIAGPKTMYAIRNAGESHQDHLTEADLIDAARELSVDLASIKAVNQV |
| | ESRGTGFTKSGKIKTLFERHIMYKKLNAKFGQAKANALAQLYPTLVNAKA |
| | GGYTGGDAELERLHGAIAIDKDCAYESASYGLFQIMGFNCVICGYDNAEE |
| | MFNDFLTGERAQLMAFVKFIKADANLWKALKDKNWAEFARRYNGPAYAQN |
| | QYDTKLAAAYKSFS [symbol] (SEQ ID NO: 42) |
| GN370 | MIDRFIRLNPTHGPRRPRRPGRRAPVRTSQRGIDLIKSFEGLRLSAYQDS |
| | VGVWTIGYGTTRGVTRYMTITVEQAERMLSNDIQRFEPELDRLAKVPLNQ |
| | NQWDALMSFVYNLGAANLASSTLLDLLNKGDYQGAADQFPHWVNAGGKRL |
| | DGLVKRRAAERALFLEPLS (SEQ ID NO: 44) |
| GN371 | MIDRFIRLNPTHRTSQRGIDLIKSFEGLRLSAYQDSVGVWTIGYGTTRGV |
| | TRYMTITVEQAERMLSNDIQRFEPELDRLAKVPLNQNQWDALMSFVYNLG |
| | AANLASSTLLDLLNKGDYQGAADQFPHWVNAGGKRLDGLVKRRAAERALF |
| | LEPLS (SEQ ID NO: 46) |
| GN394 | MAILKIGSKGLEVKNLQTSLNKIGFNLVADGIFGKATDNAVRAVQAGAGL |
| | VVDGIAGPKTMYAIRNAGESHQDHLTEADLIDAARELSVDLASIKAVNQV |
| | ESRGTGFTKSGKIKTLFERHIMYKKLNAKFGQAKANALAQLYPTLVNAKA |
| | GGYTGGDAELERLHGAIAIDKDCAYESASYGLFQIMGFNCVICGYDNAEE |
| | MFNDFLTGERAQLMAFVDFIKADANLWKALKDKNWAEFARRYNGPAYAQN |
| | QYDTKLAAAYKSFS (SEQ ID NO: 48) |
| GN396 | MAILKIGSKGLEVKNLQTSLNKIGFNLVADGIFGKATDNAVRAVQAGAGL |
| | VVDGIAGPKTMYAIRNAGESHQDHLTEADLIDAARELSVDLASIKAVNQV |
| | ESRGTGFTKSGKIKTLFERHIMYKKLNAKFGQAKANALAQLYPTLVNAKA |
| | GGYTGGDAELERLHGAIAIDKDCAYESASYGLFQIMGFNCVICGYDNAEE |
| | MFNDFLTGERAQLMAFVKFIKADANLWDALKDKNWAEFARRYNGPAYAQN |
| | QYDTKLAAAYKSFS (SEQ ID NO: 50) |
| GN408 | MAILKIGSKGLEVKNLQTSLNKIGFNLVADGIFGKATDNAVRAVQAGAGL |
| | VVDGIAGPKTMYAIRNAGESHQDHLTEADLIDAAHELSVDLASIKAVNQV |
| | ESRGTGFTKSGKIKTLFERHIMYKKLNAKFGQAKANALAQLYPTLVNAKA |
| | GGYTGGDAELERLHGAIAIDKDCAYESASYGLFQIMGFNCVICGYDNAEE |
| | MFNDFLTGERAQLMAFVKFIKADANLWKALKDKNWAEFARRYNGPAYAQN |
| | QYDTKLAAAYKSFS (SEQ ID NO: 52) |
| GN418 | MAILKIGSKGLEVKNLQTSLNDIGFNLVADGIFGKATDNAVRAVQAGAGL |
| | VVDGIAGPKTMYAIRNAGESHQDHLTEADLIDAARELSVDLASIKAVNQV |
| | ESRGTGFTKSGKIKTLFERHIMYKKLNAKFGQAKANALAQLYPTLVNAKA |
| | GGYTGGDAELERLHGAIAIDKDCAYESASYGLFQIMGFNCVICGYDNAEE |
| | MFNDFLTGERAQLMAFVKFIKADANLWKALKDKNWAEFARRYNGPAYAQN |
| | QYDTKLAAAYKSFS (SEQ ID NO: 54) |
| GN424 | MNTLRFNSRGAEVGVLQQRLVRAGYPIDVTHLYDEATEQAVKALQAAAGI |
| | VVDGIAGPNTYAVLSAGQRDRKHLTEADIARAADKLGVSPACVRAVNEVE |
| | SRGSGFLADGRPVILFERHVMYNRLVAAKRAVDAASAAQRFPNVVSAKPG |
| | GYQGGAAEYVRLDTAARIDAAIAYESASWGAFQVMGYHWERLGYSSIDEF |
| | VARMETSEGEQLDAFVRFVAADSSLRTALKNRKWAAFAKGYNGPDYARNL |
| | YDAKLAQAYERYAGTKAAA (SEQ ID NO: 56) |
| GN425 | MTLRLDDVGLDVLHLQKRLNELGANPRLLPDGQFGEVTERAVRAFQQRAG |
| | LVVDGVAGPKTMAALSGHSTSRLLGQRDLQRAADRLGVPLASVMALNAVE |
| | SRGEGFAANGRPVILFERHVMHERLQVNGLSEAEADALAARHPGLVSRRP |
| | GGYVGDTAEHQRLANARLLHDTAALESASWGLFQVMGYHWQALGYDTTQD |
| | FTERMARHEAEHLEAFVRFIEADPALHKALKGRKWAEFARRYNGPAYARN |
| | LYDVKLARAFEQFSDALQAAA (SEQ ID NO: 58) |
| GN428 | MAILKLGNRGSEVKALQQSLNKIGFSLTADGIFGKATENAVKSVQAGAGL |
| | VIDGIAGPKTFYAIRNAGDAHQEHLTEADLVDAARELGVELASMKAVNQV |
| | ESRGTGFTKTGKIKTLFERHIMYKKVTAKFGQARANALYQLYPTLVNPNS |
| | GGYIGGDAELERLQGAIALDEDCAYESASYGLFQIMGFNCQICGYSNAKE |
| | MFTDFLTGERAHLLAFVKFIKADANMWKALKNKNWAEFARRYNGPAYAKN |
| | QYDTKLAAAYKSFC (SEQ ID NO: 60) |
| GN93 | MKFFKFFKFFK [symbol] NNELPWVAEARKYIGLREDTS |
| | KTSHNPKLLAMLDRMGEFSNESRAWWHDDETPWCGLFVGYCLGVAGRYVV |
| | REWYRARAWEAPQLTKLDRPAYGALVTFTRSGGGHVGFIVGKDARGNLMV |
| | LGGNQSNAVSIAPFAVSRVTGYFWPSFWRNKTAVKSVPFEERYSLPLLKS |
| | NGELSTNEA (SEQ ID NO: 62) |
| GN431 | MAILKLGNRGTEVKALQDSLNKIGFTLVADGIFGKATENAVKTVQAGAGL |
| | VIDGIVGPKTSYAIRNAGEAHQDHLTEADLIEAANQLGVDLASVKAVNQV |
| | ESRGTGFTKSGKIKTLFERHIMYKKLMAKFGQARANAMGQMYPTLVSPVA |
| | GGYTGGDAELDRLHAAINIDEDCAYESASYGLFQIMGFNCQVCGYANAKE |
| | MFNDFLTGERAHLMAFVKFIKADAKLWQALKDKNWAEFARRYNGPAYTKN |
| | QYDTKLAAAYNSFN (SEQ ID NO: 64) |
| GN486 | M [symbol] GPRRPRRPGRRAPVRTSQRGIDLIKSFEGLRLSAYQDSVG |
| | VWTIGYGTTRGVTRYMTITVEQAERMLSNDIQRFEPELDRLAKVPLNQNQ |
| | WDALMSFVYNLGAANLASSTLLKLLNKGDYQGAADQFPRWVNAGGKRLDG |
| | LVKRRAAERALFLEPLS (SEQ ID NO: 66) |
| GN485 | MPGLSGFIRNADTPVTSLGSAGHVHVPEGPLIRINPDCLLGTPFKFFKFF |
| | KFFKFFKFFKFFKFFKNECVLL (SEQ ID NO: 68) |

In some embodiment, the lysins and/or lysin-AMP polypeptide constructs of the present disclosure are chemically modified. A chemical modification includes but is not limited to, adding chemical moieties, creating new bonds, and removing chemical moieties. Chemical modifications can occur anywhere in a lysin and/or lysin-AMP polypeptide construct, including the amino acid side chains, as well as the amino or carboxyl termini. For example, in certain embodiments, the lysin or lysin-AMP polypeptide construct comprises an N-terminal acetylation modification. In certain embodiments, the lysin or lysin-AMP polypeptide construct comprises a C-terminal amidation modification. Such modification can be present at more than one site in a lysin and/or lysin-AMP polypeptide construct.

Furthermore, one or more side groups, or terminal groups of a lysin and/or lysin-AMP polypeptide construct may be protected by protective groups known to the person ordinarily-skilled in the art.

In some embodiments, the lysins and/or lysin-AMP polypeptide constructs are conjugated to a duration enhancing moiety. In some embodiment, the duration enhancing moiety is polyethylene glycol. Polyethylene glycol (“PEG”) has been used to obtain therapeutic polypeptides of enhanced duration (Zalipsky, S., Bioconjugate Chemistry, 6:150-165 (1995); Mehvar, R., J. Pharm. Pharmaceut. Sci., 3:125-136 (2000), which is herein incorporated by reference in its entirety). The PEG backbone, (CH2CH2-0-)n, wherein n is a number of repeating monomers, is flexible and amphiphilic. When attached to another chemical entity, such as a lysin and/or lysin-AMP polypeptide construct, PEG polymer chains can protect such polypeptides from immune response and other clearance mechanisms. As a result, pegylation can lead to improved efficacy and safety by optimizing pharmacokinetics, increasing bioavailability, and decreasing immunogenicity and dosing amount and/or frequency.

### Polynucleotides

In one aspect, the present disclosure is directed an isolated polynucleotide comprising a nucleic acid molecule encoding a lysin, a variant lysin, an active fragment thereof or derivative as described herein. In some embodiments, the isolated polynucleotide sequence is a DNA sequence. In other embodiments, the isolated polynucleotide is a cDNA sequence.

In some embodiments, the isolated polynucleotide comprises a nucleic acid molecule encoding a polypeptide having at least 80%, such as at least 85%, such as at least 90%, such as at least 95%, such as at least 98%, or such as at least 99% sequence identity with a lysin, a variant lysin, an active fragment thereof or derivative as described herein, wherein the encoded polypeptide inhibits the growth, or reduces the population, or kills P. aeruginosa and optionally at least one other species of Gram-negative bacteria as described herein in the absence or presence of, or in both the absence and presence of, human serum, or in the presence of pulmonary surfactant.

In some embodiments, the isolated polynucleotide comprises a nucleic acid molecule encoding a lysin selected from GN217 (SEQ ID NO: 8), GN316 variant (SEQ ID NO: 24) GN316 (SEQ ID NO: 22), GN329 (SEQ ID NO: 26), GN333 (SEQ ID NO: 28), GN394 (SEQ ID NO: 48), GN396 (SEQ ID NO: 50), GN408 (SEQ ID NO: 52), GN418 (SEQ ID NO: 54), GN424 (SEQ ID NO: 56), GN425 (SEQ ID NO:58), GN428 (SEQ ID NO: 60), GN431 (SEQ ID NO: 64), GN486 (SEQ ID NO: 66), GN485 (SEQ ID NO: 68), Lysin PaP2_gp17 (SEQ ID NO: 96), GN123 (SEQ ID NO: 173) or GN121 (SEQ ID NO: 175) or a variant or an active fragment thereof or derivative, wherein the lysin variant or an active fragment thereof or derivative encoded by the isolated polynucleotide inhibits the growth, or reduces the population, or kills P. aeruginosa and optionally at least one other species of Gram-negative bacteria in the absence or presence of, or in both the absence and presence of, human serum, or in the presence of pulmonary surfactant. In certain embodiments, the isolated polynucleotide comprises a nucleic acid molecule encoding a lysin, variant or active fragment thereof or derivative that contains at least one modification relative to at least one of SEQ ID NOS: 8, 24, 22, 26, 28, 48, 50, 52, 54, 56, 58, 60, 64, 66, 68, 96, 173 and 175 such as at least one amino acid substitution, insertion or deletion. In certain embodiments, the isolated polynucleotide comprises a nucleic acid sequence selected from the group consisting of SEQ ID NOS: 7, 23, 21, 25, 27, 47, 49, 51, 53, 55, 57, 59, 63, 65, 67 95, 172 and 174 respectively, complements thereof or a nucleic acid sequence having at least 80%, such as at least 85%, such as at least 90%, such as at least 95%, such as at least 98%, or such as at least 99% sequence identity to one of SEQ ID NOS: 7, 23, 21, 25, 27, 47, 49, 51, 53, 55, 57, 59, 63, 65, 67 95, 172 and 174, or complements thereof, wherein the encoded polypeptide inhibits the growth, or reduces the population, or kills P. aeruginosa and optionally at least one other species of Gram-negative bacteria in the absence or presence of, or in both the absence and presence of, human serum, or in the presence of pulmonary surfactant.

In some embodiments, the isolated polynucleotide comprises a nucleic acid molecule encoding a lysin selected from at least one of GN217 lysin (SEQ ID NO: 8), GN394 lysin (SEQ ID NO: 48), GN396 lysin (SEQ ID NO: 50), GN408 lysin (SEQ ID NO: 52), GN418 lysin (SEQ ID NO: 54) and GN486 (SEQ ID NO: 66) or a variant or an active fragment thereof or derivative. In certain embodiments, the polynucleotide comprises a nucleic acid sequence selected from the group consisting of SEQ ID NOS: 7, 47, 49, 51, 53, and 65 complements thereof or a nucleic acid sequence having at least 80%, such as at least 85%, such as at least 90%, such as at least 95%, such as at least 98%, or such as at least 99% sequence identity to one of SEQ ID NOS: 77, 47, 49, 51, 53, or 65, or complements thereof, wherein the encoded polypeptide inhibits the growth, or reduces the population, or kills P. aeruginosa and optionally at least one other species of Gram-negative bacteria in the absence or presence of, or in both the absence and presence of, human serum, or in the presence of pulmonary surfactant.

In some embodiments, the isolated polynucleotide comprises a nucleic acid molecule encoding a lysin selected from at least one of GN316 (SEQ ID NO: 22), GN329 (SEQ ID NO: 26), GN333 (SEQ ID NO: 28), GN424 (SEQ ID NO: 56), GN425 (SEQ ID NO:58), GN428 (SEQ ID NO: 60), GN431 (SEQ ID NO: 64), GN485 (SEQ ID NO: 68) or a variant or an active fragment thereof or derivative, wherein the encoded polypeptide inhibits the growth, or reduces the population, or kills P. aeruginosa and optionally at least one other species of Gram-negative bacteria in the absence or presence of, or in both the absence and presence of, human serum, or in the presence of pulmonary surfactant. In certain embodiments, the variant, active fragment thereof or derivative contains at least one modification relative to at least one of SEQ ID NOS: 22, 26, 28, 56, 58, 60, 64 or 68, such as at least one amino acid substitution, insertion or deletion. In certain embodiments, the polynucleotide comprises a nucleic acid sequence selected from the group consisting of SEQ ID NOS: 21, 25, 27, 55, 57, 59, 63 and 67, complements thereof or a nucleic acid sequence having at least 80%, such as at least 85%, such as at least 90%, such as at least 95%, such as at least 98%, or such as at least 99% sequence identity to one of SEQ ID NOS: 21, 25, 27, 55, 57, 59, 63 or 67, or complements thereof, wherein the encoded polypeptide inhibits the growth, or reduces the population, or kills P. aeruginosa and optionally at least one other species of Gram-negative bacteria in the absence or presence of, or in both the absence and presence of, human serum, or in the presence of pulmonary surfactant.

In another aspect, the present disclosure is directed to an isolated polynucleotide comprising a nucleic acid molecule encoding a lysin-AMP polypeptide construct comprising:

(a) a first nucleic acid molecule encoding a first component comprising: (i) a lysin selected from the group consisting of GN76 (SEQ ID NO: 203), GN4 (SEQ ID NO: 74), GN146 (SEQ ID NO: 78), GN14 (SEQ ID NO: 124), GN37 (SEQ ID NO: 84) optionally with a single pI-increasing mutation, GN316 (SEQ ID NO: 22) optionally with a single point mutation, lysin Pap2_gp17 (SEQ ID NO: 96), GN329 (SEQ ID NO: 26), GN424 (SEQ ID NO: 56), GN202 (SEQ ID NO: 118), GN425 (SEQ ID NO: 58), GN428 (SEQ ID NO: 60), GN431 (SEQ ID NO: 64), GN486 (SEQ ID NO: 66), GN333 (SEQ ID NO: 28), and GN485 (SEQ ID NO: 68), GN123 (SEQ ID NO: 173) and GN121 (SEQ ID NO: 175); or (ii) a polypeptide having lysin activity, wherein the polypeptide is at least 80% identical to at least one of SEQ ID NOS: 203, 74, 78, 124, 84, 22, 96, 26, 56, 118, 58, 60, 64, 66, 28, 68, 173 or 175; or (iii) an active fragment of the lysin;

(b) a second nucleic acid molecule encoding a second component comprising: (i) at least one antimicrobial peptide (AMP) selected from the group consisting of Chp1 (SEQ ID NO: 133), Chp2 (SEQ ID NO: 70), CPAR39 (SEQ ID NO: 135), Chp3 (SEQ ID NO: 137), Chp4 (SEQ ID NO: 102), Chp6 (SEQ ID NO: 106), Chp7 (SEQ ID NO: 139), Chp8 (SEQ ID NO: 141), Chp9 (SEQ ID NO: 143), Chp10 (SEQ ID NO: 145), Chp11 (SEQ ID NO: 147), Chp12 (SEQ ID NO: 149), Gkh1 (SEQ ID NO: 151), Gkh2 (SEQ ID NO: 90), Unp1 (SEQ ID NO: 153), Ecp1 (SEQ ID NO: 155), Ecp2 (SEQ ID NO: 104), Tma1 (SEQ ID NO: 157), Osp1 (SEQ ID NO: 108), Unp2 (SEQ ID NO: 159), Unp3 (SEQ ID NO: 161), Gkh3 (SEQ ID NO: 163), Unp5 (SEQ ID NO: 165), Unp6 (SEQ ID NO: 167), Spi1 (SEQ ID NO: 169), Spi2 (SEQ ID NO: 171), Ecp3 (SEQ ID NO: 177), Ecp4 (SEQ ID NO: 179), ALCES1 (SEQ ID NO: 181), AVQ206 (SEQ ID NO: 183), AVQ244 (SEQ ID NO: 185), CDL907 (SEQ ID NO: 187), AGT915 (SEQ ID NO: 189), HH3930 (SEQ ID NO: 191), Fen7875 (SEQ ID NO: 193), SBR77 (SEQ ID NO: 195), Bdp1 (SEQ ID NO: 197), LVP1 (SEQ ID NO: 199), Lvp2 (SEQ ID NO: 201), an esculentin fragment (SEQ ID NO: 80), RI12 (SEQ ID NO: 88), TI15 (SEQ ID NO: 94), RI18 (SEQ ID NO: 92), FIRL (SEQ ID NO: 114), a fragment of LPS binding protein (SEQ ID NO: 76), RR12whydro (SEQ ID NO: 110), RI18 peptide derivative (SEQ ID NO: 131) and cationic peptide (SEQ ID NO: 120) or (ii) a polypeptide having AMP activity, wherein the polypeptide is at least 80% identical to at least one of SEQ ID NOS: 133, 70, 135, 137, 102, 106, 139, 141, 143, 145, 147, 149, 151, 90, 153, 155, 104, 157, 108, 159, 161, 163, 165, 167, 169, 171, 177, 179, 181, 183, 185, 187, 189, 191, 193, 195, 197, 199, 201, 80, 88, 94, 92, 114, 76, 110, 131 and 120.

In some embodiments, the isolated polynucleotides of the present disclosure comprise a nucleic acid molecule encoding a first component of a lysin-AMP construct, wherein the first component is selected from the group consisting of GN394 (SEQ ID NO: 48), GN396 (SEQ ID NO: 50), GN408 (SEQ ID NO: 52) and GN418 (SEQ ID NO: 54).

In some embodiments, the isolated polynucleotides of the present disclosure comprise a nucleic acid molecule encoding a second component of a lysin-AMP construct wherein the second component is selected from a from the group consisting of Chp1 (SEQ ID NO: 133), Chp2 (SEQ ID NO: 70), CPAR39 (SEQ ID NO: 135), Chp3 (SEQ ID NO: 137), Chp4 (SEQ ID NO: 102), Chp6 (SEQ ID NO: 106), Chp7 (SEQ ID NO: 139), Chp8 (SEQ ID NO: 141), Chp9 (SEQ ID NO: 143), Chp10 (SEQ ID NO: 145), Chp11 (SEQ ID NO: 147), Chp12 (SEQ ID NO: 149), Gkh1 (SEQ ID NO: 151), Gkh2 (SEQ ID NO: 90), Unp1 (SEQ ID NO: 153), Ecp1 (SEQ ID NO: 155), Ecp2 (SEQ ID NO: 104), Tma1 (SEQ ID NO: 157), Osp1 (SEQ ID NO: 108), Unp2 (SEQ ID NO: 159), Unp3 (SEQ ID NO: 161), Gkh3 (SEQ ID NO: 163), Unp5 (SEQ ID NO: 165), Unp6 (SEQ ID NO: 167), Spi1 (SEQ ID NO: 169), Spi2 (SEQ ID NO: 171), Ecp3 (SEQ ID NO: 177), Ecp4 (SEQ ID NO: 179), ALCES1 (SEQ ID NO: 181), AVQ206 (SEQ ID NO: 183), AVQ244 (SEQ ID NO: 185), CDL907 (SEQ ID NO: 187), AGT915 (SEQ ID NO: 189), HH3930 (SEQ ID NO: 191), Fen7875 (SEQ ID NO: 193), SBR77 (SEQ ID NO: 195), Bdp1 (SEQ ID NO: 197), LVP1 (SEQ ID NO: 199), Lvp2 (SEQ ID NO: 201), an esculentin fragment (SEQ ID NO: 80), RI12 (SEQ ID NO: 88), TI15 (SEQ ID NO: 94), RI18 (SEQ ID NO: 92), FIRL (SEQ ID NO: 114), a fragment of LPS binding protein (SEQ ID NO: 76), RR12whydro (SEQ ID NO: 110), RI18 peptide derivative (SEQ ID NO: 131) and cationic peptide (SEQ ID NO: 120) or (ii) a polypeptide having AMP activity, wherein the polypeptide is at least 80% identical to at least one of SEQ ID NOS: 133, 70, 135, 137, 102, 106, 139, 141, 143, 145, 147, 149, 151, 90, 153, 155, 104, 157, 108, 159, 161, 163, 165, 167, 169, 171, 177, 179, 181, 183, 185, 187, 189, 191, 193, 195, 197, 199, 201, 80, 88, 94, 92, 114, 76, 110, 131 and 120.

In some embodiments, isolated polynucleotides of the present disclosure further comprise a nucleic acid molecule encoding at least one structure stabilizing component of a lysin-AMP polypeptide construct to maintain at least a portion of the structure of the first and/or second component in the construct substantially the same as in the unconjugated lysin and/or AMP. In some embodiments, the present isolated polynucleotides comprise a nucleic acid molecule encoding at least one structure stabilizing component, wherein the at least one structure stabilizing component is a peptide, such as a peptide comprising glycine and/or serine residues. In one embodiment, the peptide is selected from the group consisting of TAGGTAGG (SEQ ID NO: 72), IGEM (BBa_K1485002) (SEQ ID NO: 82), PPTAGGTAGG (SEQ ID NO: 98), IGEM +PP (residues 44-58 of SEQ ID NO: 16) and AGAGAGAGAGAGAGAGAS (SEQ ID NO: 122).

More particularly, in some embodiments, the isolated polynucleotide comprises a nucleic acid molecule encoding a lysin-AMP polypeptide construct, wherein the lysin-AMP polypeptide construct is the GN168 lysin (SEQ ID NO: 2) or a polypeptide having lysin activity and having at least 80%, such as at least 85%, such as at least 90%, such as at least 95%, such as at least 98%, or such as at least 99% sequence identity to SEQ ID NO: 2.

In some embodiments, the nucleic acid molecule encoding the GN168 lysin comprises the nucleic acid sequence of SEQ ID NO: 1, a complement thereof or a nucleic acid sequence encoding a polypeptide having lysin activity and having at least 80%, such as at least 85%, such as at least 90%, such as at least 95%, such as at least 98%, or such as at least 99% sequence identity to SEQ ID NO: 1, or a complement thereof.

In some embodiments, the isolated polynucleotide comprises a nucleic acid molecule encoding a lysin-AMP polypeptide construct, wherein the lysin-AMP polypeptide construct is the GN176 lysin (SEQ ID NO: 4) or a nucleic acid molecule encoding a polypeptide having lysin activity and having at least 80%, such as at least 85%, such as at least 90%, such as at least 95%, such as at least 98%, or such as at least 99% sequence identity to SEQ ID NO: 4.

In some embodiments, the nucleic acid molecule encoding the GN176 lysin comprises the nucleic acid sequence of SEQ ID NO: 3, a complement thereof or a nucleic acid sequence encoding a polypeptide having lysin activity and having at least 80%, such as at least 85%, such as at least 90%, such as at least 95%, such as at least 98%, or such as at least 99% sequence identity to SEQ ID NO: 3, or a complement thereof.

In some embodiments, the isolated polynucleotide comprises a nucleic acid molecule encoding a lysin-AMP polypeptide construct, wherein the lysin-AMP polypeptide construct is the GN178 lysin (SEQ ID NO: 6) or a nucleic acid sequence encoding a polypeptide having at least 80%, such as at least 85%, such as at least 90%, such as at least 95%, such as at least 98%, or such as at least 99% sequence identity to SEQ ID NO: 6.

In some embodiments, the nucleic acid molecule encoding the GN178 lysin comprises the nucleic acid sequence of SEQ ID NO: 5, a complement thereof or a nucleic acid sequence encoding a polypeptide having lysin activity and having at least 80%, such as at least 85%, such as at least 90%, such as at least 95%, such as at least 98%, or such as at least 99% sequence identity to SEQ ID NO: 5, or a complement thereof.

In some embodiments, the isolated polynucleotide comprises a nucleic acid molecule encoding a lysin-AMP polypeptide construct, wherein the lysin-AMP polypeptide construct is the GN218 lysin (SEQ ID NO: 10) or a nucleic acid molecule encoding a polypeptide having lysin activity and having at least 80%, such as at least 85%, such as at least 90%, such as at least 95%, such as at least 98%, or such as at least 99% sequence identity to SEQ ID NO: 10.

In some embodiments, the nucleic acid molecule encoding the GN218 lysin comprises the nucleic acid sequence of SEQ ID NO: 9, a complement thereof or a nucleic acid sequence encoding a polypeptide having lysin activity and having at least 80%, such as at least 85%, such as at least 90%, such as at least 95%, such as at least 98%, or such as at least 99% sequence identity to SEQ ID NO: 9, or a complement thereof.

In some embodiments, the isolated polynucleotide comprises a nucleic acid molecule encoding a lysin-AMP polypeptide construct, wherein the lysin-AMP polypeptide construct is the GN223 lysin (SEQ ID NO: 12) or a nucleic acid molecule encoding a polypeptide having lysin activity and having at least 80%, such as at least 85%, such as at least 90%, such as at least 95%, such as at least 98% or such as at least 99% sequence identity to SEQ ID NO: 12.

In some embodiments, the nucleic acid molecule encoding the GN223 lysin comprises the nucleic acid sequence of SEQ ID NO: 11, a complement thereof or a nucleic acid sequence encoding a polypeptide having lysin activity and having at least 80%, such as at least 85%, such as at least 90%, such as at least 95%, such as at least 98% or such as at least 99% sequence identity to SEQ ID NO: 11, or a complement thereof.

In some embodiments, the isolated polynucleotide comprises a nucleic acid molecule encoding a lysin-AMP polypeptide construct, wherein the lysin-AMP polypeptide construct is the GN239 lysin (SEQ ID NO: 14) or a nucleic acid molecule encoding a polypeptide having lysin activity and having at least 80%, such as at least 85%, such as at least 90%, such as at least 95%, such as at least 98%, or such as at least 99% sequence identity to SEQ ID NO: 14.

In some embodiments, the nucleic acid molecule encoding the GN239 lysin comprises the nucleic acid sequence of SEQ ID NO: 13, a complement thereof or a nucleic acid sequence encoding a polypeptide having lysin activity and having at least 80%, such as at least 85%, such as at least 90%, such as at least 95%, such as at least 98%, or such as at least 99% sequence identity to SEQ ID NO: 13, or a complement thereof.

In some embodiments, the isolated polynucleotide comprises a nucleic acid molecule encoding a lysin-AMP polypeptide construct, wherein the lysin-AMP polypeptide construct is the GN243 lysin (SEQ ID NO: 16) or a nucleic acid molecule encoding a polypeptide having lysin activity and having at least 80%, such as at least 85%, such as at least 90%, such as at least 95%, such as at least 98%, or such as at least 99% sequence identity to SEQ ID NO: 16.

In some embodiments, the nucleic acid molecule encoding the GN243 lysin comprises the nucleic acid sequence of SEQ ID NO: 15, a complement thereof or a nucleic acid sequence encoding a polypeptide having lysin activity and having at least 80%, such as at least 85%, such as at least 90%, such as at least 95%, such as at least 98%, or such as at least 99% sequence identity to SEQ ID NO: 15, or a complement thereof.

In some embodiments, the isolated polynucleotide comprises a nucleic acid molecule encoding a lysin-AMP polypeptide construct, wherein the lysin-AMP polypeptide construct is the GN280 lysin (SEQ ID NO: 18) or a nucleic acid molecule encoding a polypeptide having lysin activity and having at least 80%, such as at least 85%, such as at least 90%, such as at least 95%, such as at least 98%, or such as at least 99% sequence identity to SEQ ID NO: 18.

In some embodiments, the nucleic acid molecule encoding the GN280 lysin comprises the nucleic acid sequence of SEQ ID NO: 17, a complement thereof or a nucleic acid sequence encoding a polypeptide having lysin activity and having at least 80%, such as at least 85%, such as at least 90%, such as at least 95%, such as at least 98%, or such as at least 99% sequence identity to SEQ ID NO: 17, or a complement thereof.

In some embodiments, the isolated polynucleotide comprises a nucleic acid molecule encoding a lysin-AMP polypeptide construct, wherein the lysin-AMP polypeptide construct is the GN281 lysin (SEQ ID NO: 20) or a nucleic acid molecule encoding a polypeptide having lysin activity and having at least 80%, such as at least 85%, such as at least 90%, such as at least 95%, such as at least 98%, or such as at least 99% sequence identity to SEQ ID NO: 20.

In some embodiments, the nucleic acid molecule encoding the GN281 lysin comprises the nucleic acid sequence of SEQ ID NO: 19, a complement thereof or a nucleic acid sequence encoding a polypeptide having lysin activity and having at least 80%, such as at least 85%, such as at least 90%, such as at least 95%, such as at least 98%, or such as at least 99% sequence identity to SEQ ID NO: 19, or a complement thereof.

In some embodiments, the isolated polynucleotide comprises a nucleic acid molecule encoding a lysin-AMP polypeptide construct, wherein the lysin-AMP polypeptide construct is the GN349 lysin (SEQ ID NO: 30) or a nucleic acid molecule encoding a polypeptide having lysin activity and having at least 80%, such as at least 85%, such as at least 90%, such as at least 95%, such as at least 98%, or such as at least 99% sequence identity to SEQ ID NO: 30.

In some embodiments, the nucleic acid molecule encoding the GN349 lysin comprises the nucleic acid sequence of SEQ ID NO: 29, a complement thereof or a nucleic acid sequence encoding a polypeptide having lysin activity and having at least 80%, such as at least 85%, such as at least 90%, such as at least 95%, such as at least 98%, or such as at least 99% sequence identity to SEQ ID NO: 29, or a complement thereof.

In some embodiments, the isolated polynucleotide comprises a nucleic acid molecule encoding a lysin-AMP polypeptide construct, wherein the lysin-AMP polypeptide construct is the GN351 lysin (SEQ ID NO: 32) or a nucleic acid molecule encoding a polypeptide having lysin activity and having at least 80%, such as at least 85%, such as at least 90%, such as at least 95%, such as at least 98%, or such as at least 99% sequence identity to SEQ ID NO: 32.

In some embodiments, the nucleic acid molecule encoding the GN351 lysin comprises the nucleic acid sequence of SEQ ID NO: 31, a complement thereof or a nucleic acid sequence encoding a polypeptide having lysin activity and having at least 80%, such as at least 85%, such as at least 90%, such as at least 95%, such as at least 98%, or such as at least 99% sequence identity to SEQ ID NO: 31, or a complement thereof.

In some embodiments, the isolated polynucleotide comprises a nucleic acid molecule encoding a lysin-AMP polypeptide construct, wherein the lysin-AMP polypeptide construct is the GN352 lysin (SEQ ID NO: 34) or a nucleic acid molecule encoding a polypeptide having lysin activity and having at least 80%, such as at least 85%, such as at least 90%, such as at least 95%, such as at least 98%, or such as at least 99% sequence identity to SEQ ID NO: 34.

In some embodiments, the nucleic acid molecule encoding the GN352 lysin comprises the nucleic acid sequence of SEQ ID NO: 33, a complement thereof or a nucleic acid sequence encoding a polypeptide having lysin activity and having at least 80%, such as at least 85%, such as at least 90%, such as at least 95%, such as at least 98%, or such as at least 99% sequence identity to SEQ ID NO: 33, or a complement thereof.

In some embodiments, the isolated polynucleotide comprises a nucleic acid molecule encoding a lysin-AMP polypeptide construct, wherein the lysin-AMP polypeptide construct is the GN353 lysin (SEQ ID NO: 36) or a nucleic acid molecule encoding a polypeptide having lysin activity and having at least 80%, such as at least 85%, such as at least 90%, such as at least 95%, such as at least 98%, or such as at least 99% sequence identity to SEQ ID NO: 36.

In some embodiments, the nucleic acid molecule encoding the GN353 lysin comprises the nucleic acid sequence of SEQ ID NO: 35, a complement thereof or a nucleic acid sequence encoding a polypeptide having lysin activity and having at least 80%, such as at least 85%, such as at least 90%, such as at least 95%, such as at least 98%, or such as at least 99% sequence identity to SEQ ID NO: 35, or a complement thereof.

In some embodiments, the isolated polynucleotide comprises a nucleic acid molecule encoding a lysin-AMP polypeptide construct, wherein the lysin-AMP polypeptide construct is the GN357 lysin (SEQ ID NO: 38) or a nucleic acid molecule encoding a polypeptide having lysin activity and having at least 80%, such as at least 85%, such as at least 90%, such as at least 95%, such as at least 98%, or such as at least 99% sequence identity to SEQ ID NO: 38.

In some embodiments, the nucleic acid molecule encoding the GN357 lysin comprises the nucleic acid sequence of SEQ ID NO: 37, a complement thereof or a nucleic acid sequence encoding a polypeptide having lysin activity and having at least 80%, such as at least 85%, such as at least 90%, such as at least 95%, such as at least 98%, or such as at least 99% sequence identity to SEQ ID NO: 37, or a complement thereof.

In some embodiments, the isolated polynucleotide comprises a nucleic acid molecule encoding a lysin-AMP polypeptide construct, wherein the lysin-AMP polypeptide construct is the GN359 lysin (SEQ ID NO: 40) or a nucleic acid molecule encoding a polypeptide having lysin activity and having at least 80%, such as at least 85%, such as at least 90%, such as at least 95%, such as at least 98%, or such as at least 99% sequence identity to SEQ ID NO: 40.

In some embodiments, the nucleic acid molecule encoding the GN359 lysin comprises the nucleic acid sequence of SEQ ID NO: 39, a complement thereof or a nucleic acid sequence encoding a polypeptide having lysin activity and having at least 80%, such as at least 85%, such as at least 90%, such as at least 95%, such as at least 98%, or such as at least 99% sequence identity to SEQ ID NO: 39, or a complement thereof.

In some embodiments, the isolated polynucleotide comprises a nucleic acid molecule encoding a lysin-AMP polypeptide construct, wherein the lysin-AMP polypeptide construct is the GN369 lysin (SEQ ID NO: 42) or a nucleic acid molecule encoding a polypeptide having lysin activity and having at least 80%, such as at least 85%, such as at least 90%, such as at least 95%, such as at least 98%, or such as at least 99% sequence identity to SEQ ID NO: 42.

In some embodiments, the nucleic acid molecule encoding the GN369 lysin comprises the nucleic acid sequence of SEQ ID NO: 41, a complement thereof or a nucleic acid sequence encoding a polypeptide having lysin activity and having at least 80%, such as at least 85%, such as at least 90%, such as at least 95%, such as at least 98%, or such as at least 99% sequence identity to SEQ ID NO: 41, or a complement thereof.

In some embodiments, the isolated polynucleotide comprises a nucleic acid molecule encoding a lysin-AMP polypeptide construct, wherein the lysin-AMP polypeptide construct is the GN370 lysin (SEQ ID NO: 44) or a nucleic acid molecule encoding a polypeptide having lysin activity and having at least 80%, such as at least 85%, such as at least 90%, such as at least 95%, such as at least 98%, or such as at least 99% sequence identity to SEQ ID NO: 44.

In some embodiments, the nucleic acid molecule encoding the GN370 lysin comprises the nucleic acid sequence of SEQ ID NO: 43, a complement thereof or a nucleic acid sequence encoding a polypeptide having lysin activity and having at least 80%, such as at least 85%, such as at least 90%, such as at least 95%, such as at least 98%, or such as at least 99% sequence identity to SEQ ID NO: 43, or a complement thereof.

In some embodiments, the isolated polynucleotide comprises a nucleic acid molecule encoding a lysin-AMP polypeptide construct, wherein the lysin-AMP polypeptide construct is the GN371 lysin (SEQ ID NO: 46) or a nucleic acid molecule encoding a polypeptide having lysin activity and having at least 80%, such as at least 85%, such as at least 90%, such as at least 95%, such as at least 98%, or such as at least 99% sequence identity to SEQ ID NO: 46.

In some embodiments, the nucleic acid molecule encoding the GN371 lysin comprises the nucleic acid sequence of SEQ ID NO: 45, a complement thereof or a nucleic acid sequence encoding a polypeptide having lysin activity and having at least 80%, such as at least 85%, such as at least 90%, such as at least 95%, such as at least 98%, or such as at least 99% sequence identity to SEQ ID NO: 45, or a complement thereof.

In some embodiments, the isolated polynucleotide comprises a nucleic acid molecule encoding a lysin-AMP polypeptide construct, wherein the lysin-AMP polypeptide construct is the GN93 lysin (SEQ ID NO: 62) or a nucleic acid molecule encoding a polypeptide having lysin activity and having at least 80%, such as at least 85%, such as at least 90%, such as at least 95%, such as at least 98%, or such as at least 99% sequence identity to SEQ ID NO: 62.

In some embodiments, the nucleic acid molecule encoding the GN93 comprises the nucleic acid sequence of SEQ ID NO: 61, a complement thereof or a nucleic acid sequence encoding a polypeptide having lysin activity and having at least 80%, such as at least 85%, such as at least 90%, such as at least 95%, such as at least 98%, or such as at least 99% sequence identity to SEQ ID NO: 61, or a complement thereof.

### Vectors and Host Cells

In another aspect, the present disclosure is directed to a vector comprising an isolated polynucleotide comprising a nucleic acid molecule encoding any of the lysin-AMP polypeptides, lysin polypeptides, variants, active fragments thereof or derivatives disclosed herein or a complementary sequence of the present isolated polynucleotides. In some embodiments, the vector is a plasmid or cosmid. In other embodiments, the vector is a viral vector, wherein additional DNA segments can be ligated into the viral vector. In some embodiments, the vector can autonomously replicate in a host cell into which it is introduced. In some embodiments, the vector can be integrated into the genome of a host cell upon introduction into the host cell and thereby be replicated along with the host genome.

In some embodiments, particular vectors, referred to herein as “recombinant expression vectors” or “expression vectors”, can direct the expression of genes to which they are operatively linked. A polynucleotide sequence is “operatively linked” when it is placed into a functional relationship with another nucleotide sequence. For example, a promoter or regulatory DNA sequence is said to be “operatively linked” to a DNA sequence that codes for an RNA and/or a protein if the two sequences are operatively linked, or situated such that the promoter or regulatory DNA sequence affects the expression level of the coding or structural DNA sequence. Operatively linked DNA sequences are typically, but not necessarily, contiguous.

Generally, any system or vector suitable to maintain, propagate or express a polypeptide in a host may be used for expression of the present lysin-AMP polypeptides, lysin polypeptides, variants, active fragments thereof or derivatives. The appropriate DNA/polynucleotide sequence may be inserted into the expression system by any of a variety of well-known and routine techniques, such as, for example, those set forth in Sambrook et al., eds., Molecular Cloning: A Laboratory Manual (3rd Ed.), Vols. 1-3, Cold Spring Harbor Laboratory (2001). Additionally, tags can also be added to the lysin-AMP polypeptides, lysin polypeptides, variants, active fragments thereof or derivatives of the present disclosure to provide convenient methods of isolation, e.g., c-myc, biotin, poly-His, etc. Kits for such expression systems are commercially available.

A wide variety of host/expression vector combinations may be employed in expressing the polynucleotide sequences encoding the present lysin-AMP polypeptides, lysin polypeptides, variants, active fragments thereof or derivatives. Large numbers of suitable vectors are known to those of skill in the art, and are commercially available. Examples of suitable vectors are provided, e.g., in Sambrook et al, eds., Molecular Cloning: A Laboratory Manual (3rd Ed.), Vols. 1-3, Cold Spring Harbor Laboratory (2001). Such vectors include, among others, chromosomal, episomal and virus derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids.

Furthermore, the vectors may provide for the constitutive or inducible expression of the lysin-AMP polypeptides, lysin polypeptides, variants, active fragments thereof or derivatives of the present disclosure. Suitable vectors include but are not limited to derivatives of SV40 and known bacterial plasmids, e.g., E. coli plasmids colE1, pCR1, pBR322, pMB9 and their derivatives, plasmids such as RP4, pBAD24 and pBAD-TOPO; phage DNAS, e.g., the numerous derivatives of phage A, e.g., NM989, and other phage DNA, e.g., M13 and filamentous single stranded phage DNA; yeast plasmids such as the 2 D plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like. Many of the vectors mentioned above are commercially available from vendors such as New England Biolabs Inc., Addgene, Takara Bio Inc., ThermoFisher Scientific Inc., etc.

Additionally, vectors may comprise various regulatory elements (including promoter, ribosome binding site, terminator, enhancer, various cis-elements for controlling the expression level) wherein the vector is constructed in accordance with the host cell. Any of a wide variety of expression control sequences (sequences that control the expression of a polynucleotide sequence operatively linked to it) may be used in these vectors to express the polynucleotide sequences encoding the lysin-AMP polypeptides, lysin polypeptides, variants, active fragments thereof or derivatives thereof of the present disclosure. Useful control sequences include, but are not limited to: the early or late promoters of SV40, CMV, vaccinia, polyoma or adenovirus, the lac system, the trp system, the TAC system, the TRC system, the LTR system, the major operator and promoter regions of phage A, the control regions of fd coat protein, the promoter for 3-phosphoglycerate kinase or other glycolytic enzymes, the promoters of acid phosphatase (e.g., Pho5), the promoters of the yeast-mating factors, E. coli promoter for expression in bacteria, and other promoter sequences known to control the expression of genes of prokaryotic or eukaryotic cells or their viruses, and various combinations thereof. Typically, the polynucleotide sequences encoding the lysin-AMP polypeptides, lysin polypeptides, variants, active fragments thereof or derivatives is operatively linked to a heterologous promoter or regulatory element.

In another aspect, the present disclosure is directed to a host cell comprising any of the vectors disclosed herein including the expression vectors comprising the polynucleotide sequences encoding the lysin-AMP polypeptides, lysin polypeptides, variants, active fragments thereof or derivatives of the present disclosure. A wide variety of host cells are useful in expressing the present polypeptides. Non-limiting examples of host cells suitable for expression of the present polypeptides include well known eukaryotic and prokaryotic hosts, such as strains of E. coli, Pseudomonas, Bacillus, Streptomyces, fungi such as yeasts, and animal cells, such as CHO, R1.1, B—W and L-M cells, African Green Monkey kidney cells (e.g., COS 1, COS 7, BSC1, BSC40, and BMT10), insect cells (e.g., Sf9), and human cells and plant cells in tissue culture. While the expression host may be any known expression host cell, in a typical embodiment the expression host is one of the strains of E. coli. These include, but are not limited to commercially available E. coli strains such as Top10 (ThermoFisher Scientific, Inc.), DH5a (Thermo Fisher Scientific, Inc.), XLI-Blue (Agilent Technologies, Inc.), SCSllO (Agilent Technologies, Inc.), JM109 (Promega, Inc.), LMG194 (ATCC), and BL21 (Thermo Fisher Scientific, Inc.).

There are several advantages of using E. coli as a host system including: fast growth kinetics, where under the optimal environmental conditions, its doubling time is about 20 min (Sezonov et al., J. Bacterial. 189 8746-8749 (2007)), easily achieved high density cultures, easy and fast transformation with exogenous DNA, etc. Details regarding protein expression in E. coli, including plasmid selection as well as strain selection are discussed in details by Rosano, G. and Ceccarelli, E., Front Microbial., 5: 172 (2014).

Efficient expression of the present lysin-AMP polypeptides, lysin polypeptides, variants, active fragments thereof or derivatives depends on a variety of factors such as optimal expression signals (both at the level of transcription and translation), correct protein folding, and cell growth characteristics. Regarding methods for constructing the vector and methods for transducing the constructed recombinant vector into the host cell, conventional methods known in the art can be utilized. While it is understood that not all vectors, expression control sequences, and hosts will function equally well to express the polynucleotide sequences encoding lysin-AMP polypeptides, lysin polypeptides, variants, active fragments thereof or derivatives of the present disclosure, one skilled in the art will be able to select the proper vectors, expression control sequences, and hosts without undue experimentation to accomplish the desired expression without departing from the scope of this disclosure.

In some embodiments, the present inventors have found a correlation between level of expression and activity of the expressed polypeptide; in E. coli expression systems in particular, moderate levels of expression (for example between about 1 and 10 mg/liter) have produced lysin-AMP polypeptides, lysin polypeptides, variants, active fragments thereof or derivatives with higher levels of activity than those that were expressed at higher levels in E. coli (for example between about 20 and about 100 mg/liter), the latter having sometimes produced wholly inactive polypeptides.

Lysin-AMP polypeptides, lysin polypeptides, variants, active fragments thereof or derivatives of the present disclosure can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. High performance liquid chromatography can also employed for lysin polypeptide purification.

Alternatively, the vector system used for the production of lysin-AMP polypeptides, lysin polypeptides, variants, active fragments thereof or derivatives of the present disclosure may be a cell-free expression system. Various cell-free expression systems are commercially available, including, but are not limited to those available from Promega, LifeTechnologies, Clonetech, etc.

As indicated above, there is an array of choices when it comes to protein production and purification. Examples of suitable methods and strategies to be considered in protein production and purification are provided in WO 2017/049233, which is herein incorporated by reference in its entirety and further provided in Structural Genomics Consortium et al., Nat. Methods., 5(2): 135-146 (2008).

### Pharmaceutical Compositions

In another aspect, the present disclosure is directed to a pharmaceutical composition comprising an effective amount of lysin-AMP polypeptides, lysin polypeptides, variants, active fragments thereof or derivatives as described herein and a pharmaceutically acceptable carrier. In some embodiments, the present pharmaceutical composition comprises at least one activity selected from inhibiting P. aeruginosa bacterial growth, reducing a P. aeruginosa bacterial population and/or killing P. aeruginosa in the absence and/or presence of human serum, or in the presence of pulmonary surfactant.

In some embodiments, the present pharmaceutical compositions further comprise one or more antibiotics suitable for the treatment of Gram-negative bacteria. Typical antibiotics include one or more of ceftazidime, cefepime, cefoperazone, ceftobiprole, ciprofloxacin, levofloxacin, aminoglycosides, imipenem, meropenem, doripenem, gentamicin, tobramycin, amikacin, piperacillin, ticarcillin, penicillin, rifampicin, polymyxin B, and colistin. Additional suitable antibiotics are described in Table 3.

In some embodiments, the pharmaceutical composition is a solution, a suspension, an emulsion, an inhalable powder, an aerosol, or a spray. The pharmaceutical compositions of the present disclosure can take the form of solutions, suspensions, emulsion, tablets, pills, pellets, capsules, capsules containing liquids, powders, sustained-release formulations, suppositories, tampon applications emulsions, aerosols, sprays, suspensions, lozenges, troches, candies, injectants, chewing gums, ointments, smears, time-release patches, liquid absorbed wipes, and combinations thereof.

Administration of the pharmaceutical compositions of the present disclosure may be topical, i.e., the pharmaceutical composition is applied directly where its action is desired (for example directly to a wound). The topical compositions of the present disclosure may further comprise a pharmaceutically or physiologically acceptable carrier, such as a dermatologically or an otically acceptable carrier. Such carriers, in the case of dermatologically acceptable carriers, are preferably compatible with skin, nails, mucous membranes, tissues and/or hair, and can include any conventionally used dermatological carrier meeting these requirements. In the case of otically acceptable carriers, the carrier is preferably compatible with all parts of the ear. Such carriers can be readily selected by one of ordinary skill in the art.

Carriers for topical administration of the lysin, active fragment thereof and/or lysin-AMP polypeptide construct of the present disclosure include, but are not limited to, mineral oil, liquid petroleum, white petroleum, propylene glycol, polyoxyethylene and/or polyoxypropylene compounds, emulsifying wax, sorbitan monostearate, polysorbate 60, cetyl esters wax, cetearyl alcohol, 2-octyldodecanol, benzyl alcohol, and water. In formulating skin ointments, the active components of the present disclosure may be formulated in an oleaginous hydrocarbon base, an anhydrous absorption base, a water-in-oil absorption base, an oil-in-water water-removable base and/or a water-soluble base. In formulating otic compositions, the active components of the present disclosure may be formulation in an aqueous polymeric suspension including such carriers as dextrans, polyethylene glycols, polyvinylpyrrolidone, polysaccharide gels, Gelrite®, cellulosic polymers like hydroxypropyl methylcellulose, and carboxy-containing polymers such as polymers or copolymers of acrylic acid, as well as other polymeric demulcents.

The topical compositions according to the present disclosure may be in any form suitable for topical application, including aqueous, aqueous-alcoholic or oily solutions, lotion or serum dispersions, aqueous, anhydrous or oily gels, emulsions obtained by dispersion of a fatty phase in an aqueous phase (OAV or oil in water) or, conversely, (W/O or water in oil), microemulsions or alternatively microcapsules, microparticles or lipid vesicle dispersions of ionic and/or nonionic type, creams, lotions, gels, foams (which will generally require a pressurized canister, a suitable applicator an emulsifier and an inert propellant), essences, milks, suspensions, or patches. Topical compositions of the present disclosure may also contain adjuvants such as hydrophilic or lipophilic gelling agents, hydrophilic or lipophilic active agents, preserving agents, antioxidants, solvents, fragrances, fillers, sunscreens, odor-absorbers and dyestuffs. In a further aspect, the topical compositions may be administered in conjunction with devices such as transdermal patches, dressings, pads, wraps, matrices and bandages capable of being adhered to or otherwise associated with the skin or other tissue of a subject, being capable of delivering a therapeutically effective amount of one or more antibacterial peptides in accordance with the present disclosure.

In one embodiment, the topical compositions of the present disclosure additionally comprise one or more components used to treat topical burns. Such components typically include, but are not limited to, a propylene glycol hydrogel; a combination of a glycol, a cellulose derivative and a water soluble aluminum salt; an antiseptic; an antibiotic; and a corticosteroid. Humectants (such as solid or liquid wax esters), absorption promoters (such as hydrophilic clays, or starches), viscocity building agents, and skin-protecting agents may also be added. Topical formulations may be in the form of rinses such as mouthwash. See, e.g., WO2004/004650.

In some embodiments, administration of the pharmaceutical compositions of the present disclosure may be systemic. Systemic administration can be enteral or oral, i.e., a substance is given via the digestive tract, parenteral, i.e., a substance is given by other routes than the digestive tract such as by injection or inhalation. Thus, the polypeptides including lysin-AMP polypeptides, lysin polypeptides, variants, active fragments thereof or derivatives of the present disclosure can be administered to a subject orally, parenterally, by inhalation, topically, rectally, nasally, buccally or via an implanted reservoir or by any other known method. The lysin-AMP polypeptides, lysin polypeptides, variants, active fragments thereof or derivatives of the present disclosure can also be administered by means of sustained release dosage forms.

For oral administration, the lysin-AMP polypeptides, lysin polypeptides, variants, active fragments thereof or derivatives of the present disclosure can be formulated into solid or liquid preparations, for example tablets, capsules, powders, solutions, suspensions and dispersions. The lysin, active fragment thereof and/or lysin-AMP polypeptide constructs can be formulated with excipients such as, e.g., lactose, sucrose, corn starch, gelatin, potato starch, alginic acid and/or magnesium stearate.

For preparing solid compositions such as tablets and pills, lysin-AMP polypeptides, lysin polypeptides, variants, active fragments thereof or derivatives of the present disclosure is mixed with a pharmaceutical excipient to form a solid pre-formulation composition. If desired, tablets may be sugar coated or enteric coated by standard techniques. The tablets or pills may be coated or otherwise compounded to provide a dosage form affording the advantage of prolonged action. For example, the tablet or pill can include an inner dosage and an outer dosage component, the latter being in the form of an envelope over the former. The two dosage components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The pharmaceutical compositions of the present disclosure may also be administered by injection. For example, the pharmaceutical compositions can be administered intramuscularly, intrathecally, subdermally, subcutaneously, or intravenously to treat infections by Gram-negative bacteria, more specifically those caused by P. aeruginosa. The pharmaceutically acceptable carrier may be comprised of distilled water, a saline solution, albumin, a serum, or any combinations thereof. Additionally, pharmaceutical compositions of parenteral injections can comprise pH buffered solutions, adjuvants (e.g., preservatives, wetting agents, emulsifying agents, and dispersing agents), liposomal formulations, nanoparticles, dispersions, suspensions or emulsions as well as sterile powders for reconstitution into sterile injectable solutions or dispersions just prior to use.

In cases where parenteral injection is the chosen mode of administration, an isotonic formulation is preferably used. Generally, additives for isotonicity can include sodium chloride, dextrose, mannitol, sorbitol, and lactose. In some cases, isotonic solutions such as phosphate buffered saline are preferred. Stabilizers can include gelatin and albumin. A vasoconstriction agent can be added to the formulation. The pharmaceutical preparations according to this type of application are provided sterile and pyrogen free.

In another embodiment, the pharmaceutical compositions of the present disclosure are inhalable compositions. In some embodiments, the present pharmaceutical compositions are advantageously formulated as a dry, inhalable powder. In specific embodiments, the present pharmaceutical compositions may further be formulated with a propellant for aerosol delivery. Examples of suitable propellants include, but are not limited to: dichlorodifluoromethane, trichlorofluoromethane, dichloro-tetrafluoroethane and carbon dioxide. In certain embodiments, the formulations may be nebulized.

A surfactant can be added to an inhalable pharmaceutical composition of the present disclosure in order to lower the surface and interfacial tension between the medicaments and the propellant. The surfactant may be any suitable, non-toxic compound which is non-reactive with the present polypeptides.

Examples of suitable surfactants include, but are not limited to: oleic acid; sorbitan trioleate; cetyl pyridinium chloride; soya lecithin; polyoxyethylene(20) sorbitan monolaurate; polyoxyethylene (10) stearyl ether; polyoxyethylene (2) oleyl ether; polyoxypropylene-polyoxyethylene ethylene diamine block copolymers; polyoxyethylene(20) sorbitan monostearate; polyoxyethylene(20) sorbitan monooleate; polyoxypropylene-polyoxyethylene block copolymers; castor oil ethoxylate; and combinations thereof.

In some embodiments, the inhalable pharmaceutical compositions include excipients. Examples of suitable excipients include, but are not limited to: lactose, starch, propylene glycol diesters of medium chain fatty acids; triglyceride esters of medium chain fatty acids, short chains, or long chains, or any combination thereof; perfluorodimethylcyclobutane; perfluorocyclobutane; polyethylene glycol; menthol; lauroglycol; diethylene glycol monoethylether; polyglycolized glycerides of medium chain fatty acids; alcohols; eucalyptus oil; short chain fatty acids; and combinations thereof.

In some embodiments, the pharmaceutical compositions of the present disclosure comprise nasal formulations. Nasal formulations include, for instance, nasal sprays, nasal drops, nasal ointments, nasal washes, nasal injections, nasal packings, bronchial sprays and inhalers, or indirectly through use of throat lozenges, mouthwashes or gargles, or through the use of ointments applied to the nasal nares, or the face or any combination of these and similar methods of application.

In another embodiment, the pharmaceutical compositions of the present disclosure comprise a complementary agent, including one or more antimicrobial agents and/or one or more conventional antibiotics. In order to accelerate the treatment of the infection, or augment the antibacterial effect, the therapeutic agent containing the lysin-AMP polypeptides, lysin polypeptides, variants, active fragments thereof or derivatives of the present disclosure may further include at least one complementary agent that can also potentiate the bactericidal activity of the peptide. The complementary agent may be one or more antibiotics used to treat Gram-negative bacteria. In one embodiment, the complementary agent is an antibiotic or antimicrobial agent used for the treatment of infections caused by P. aeruginosa.

The pharmaceutical compositions of the present disclosure may be presented in unit dosage form and may be prepared by any methods well known in the art. The amount of active ingredients which can be combined with a carrier material to produce a single dosage form will vary depending upon the host being treated, the duration of exposure of the recipient to the infectious bacteria, the size and weight of the subject, and the particular mode of administration. The amount of active ingredients that can be combined with a carrier material to produce a single dosage form will generally be that amount of each compound which produces a therapeutic effect. Generally, out of one hundred percent, the total amount will range from about 1 percent to about ninety-nine percent of active ingredients, preferably from about 5 percent to about 70 percent, most preferably from about 10 percent to about 30 percent.

### Dosage and Administration

Dosages administered depend on a number of factors including the activity of infection being treated, the age, health and general physical condition of the subject to be treated, the activity of a particular lysin-AMP polypeptide, lysin polypeptide, variant, active fragment thereof or derivative, the nature and activity of the antibiotic if any with which a lysin-AMP polypeptide, lysin polypeptide, variant, active fragment thereof or derivative according to the present disclosure is being paired and the combined effect of such pairing. Generally, effective amounts of the present lysin-AMP polypeptide, lysin polypeptide, variant, active fragment thereof or derivative to be administered are anticipated to fall within the range of 1-50 mg/kg (or 1 to 50 mcg/ml) administered 1-4 times daily for a period up to 14 days. The antibiotic if one is also used will be administered at standard dosing regimens or in lower amounts in view of the synergy. All such dosages and regimens however (whether of the lysin-AMP polypeptide, lysin polypeptide, variant, active fragment thereof or derivative or any antibiotic administered in conjunction therewith) are subject to optimization. Optimal dosages can be determined by performing in vitro and in vivo pilot efficacy experiments as is within the skill of the art but taking the present disclosure into account.

It is contemplated that the present lysin-AMP polypeptides, lysin polypeptides, variants, active fragments thereof or derivatives provide a bactericidal and, when used in smaller amounts, bacteriostatic effect, and are active against a range of antibiotic-resistant bacteria and are not associated with evolving resistance. Based on the present disclosure, in a clinical setting, the present lysin-AMP polypeptides, lysin polypeptides, variants, active fragments thereof or derivatives are a potent alternative (or additive or component) of compositions for treating infections arising from drug- and multidrug-resistant bacteria alone or together with antibiotics (even antibiotics to which resistance has developed). Existing resistance mechanisms for Gram-negative bacteria should not affect sensitivity to the lytic activity of the present polypeptides.

In some embodiments, time exposure to the present lysin-AMP polypeptides, lysin polypeptides, variants, active fragments thereof or derivatives may influence the desired concentration of active polypeptide units per ml. Carriers that are classified as “long” or “slow” release carriers (such as, for example, certain nasal sprays or lozenges) could possess or provide a lower concentration of polypeptide units per ml, but over a longer period of time, whereas a “short” or “fast” release carrier (such as, for example, a gargle) could possess or provide a high concentration polypeptide units (mcg) per ml, but over a shorter period of time. There are circumstances where it may be necessary to have a much higher unit/ml dosage or a lower unit/ml dosage.

For any polypeptide of the present disclosure, the therapeutically effective dose can be estimated initially either in cell culture assays or in animal models, usually mice, rabbits, dogs, or pigs. The animal model can also be used to achieve a desirable concentration range and route of administration. Obtained information can then be used to determine the effective doses, as well as routes of administration in humans. Dosage and administration can be further adjusted to provide sufficient levels of the active ingredient or to maintain the desired effect. Additional factors which may be taken into account include the severity of the disease state, age, weight and gender of the patient; diet, desired duration of treatment, method of administration, time and frequency of administration, drug combination(s), reaction sensitivities, and tolerance/response to therapy and the judgment of the treating physician.

A treatment regimen can entail daily administration (e.g., once, twice, thrice, etc. daily), every other day (e.g., once, twice, thrice, etc. every other day), semi-weekly, weekly, once every two weeks, once a month, etc. In one embodiment, treatment can be given as a continuous infusion. Unit doses can be administered on multiple occasions. Intervals can also be irregular as indicated by monitoring clinical symptoms. Alternatively, the unit dose can be administered as a sustained release formulation, in which case less frequent administration is required. Dosage and frequency may vary depending on the patient. It will be understood by one of skill in the art that such guidelines will be adjusted for localized administration, e.g. intranasal, inhalation, rectal, etc., or for systemic administration, e.g. oral, rectal (e.g., via enema), i.m. (intramuscular), i.p. (intraperitoneal), i.v. (intravenous), s.c. (subcutaneous), transurethral, and the like.

### Methods

In another aspect, the present disclosure is directed to a method of treating a bacterial infection caused by P. aeruginosa and optionally one or more additional species of Gram-negative bacteria as described herein, comprising administering to a subject diagnosed with, at risk for, or exhibiting symptoms of a bacterial infection, a pharmaceutical composition as herein described. In one aspect, the bacterial infection is an infection of an organ or tissue in which pulmonary surfactant is present.

The terms “infection” and “bacterial infection” are meant to include respiratory tract infections (RTIs), such as respiratory tract infections in patients having cystic fibrosis (CF), lower respiratory tract infections, such as acute exacerbation of chronic bronchitis (ACEB), acute sinusitis, community-acquired pneumonia (CAP), hospital-acquired pneumonia (HAP) and nosocomial respiratory tract infections; sexually transmitted diseases, such as gonococcal cervicitis and gonococcal urethritis; urinary tract infections; acute otitis media; sepsis including neonatal septisemia and catheter-related sepsis; and osteomyelitis. Infections caused by drug-resistant bacteria and multidrug-resistant bacteria are also contemplated.

Non-limiting examples of infections caused by P. aeruginosa include: A) Nosocomial infections: 1. Respiratory tract infections especially in cystic fibrosis patients and mechanically-ventilated patients; 2. Bacteraemia and sepsis; 3. Wound infections, particularly those of burn victims; 4. Urinary tract infections; 5. Post-surgery infections on invasive devises; 6. Endocarditis by intravenous administration of contaminated drug solutions; 7. Infections in patients with acquired immunodeficiency syndrome, cancer chemotherapy, steroid therapy, hematological malignancies, organ transplantation, renal replacement therapy, and other conditions with severe neutropenia. B) Community-acquired infections: 1. Community-acquired respiratory tract infections; 2. Meningitis; 3. Folliculitis and infections of the ear canal caused by contaminated water; 4. Malignant otitis externa in the elderly and diabetics; 5. Osteomyelitis of the caleaneus in children; 6. Eye infections commonly associated with contaminated contact lens; 7. Skin infections such as nail infections in people whose hands are frequently exposed to water; 8. Gastrointestinal tract infections; and 9. Muscoskeletal system infections.

The one or more additional species of Gram-negative bacteria of the present methods may include any of the additional species of Gram-negative bacteria as described herein. Typically, the additional species of Gram-negative bacteria are selected from one or more of Acinetobacter baumannii, Acinetobacter haemolyticus, Actinobacillus actinomycetemcomitans, Aeromonas hydrophila, Bacteroides spp., such as, Bacteroides fragilis, Bacteroides theataioatamicron, Bacteroides distasonis, Bacteroides ovatus, Bacteroides vulgatus, Bartonella Quintana, Bordetella pertussis, Brucella spp., such as, Brucella melitensis, Burkholderia spp, such as, Burkholderia cepacia, Burkholderia pseudomallei, and Burkholderia mallei, Fusobacterium, Prevotella corporis, Prevotella intermedia, Prevotella endodontalis, Porphyromonas asaccharolytica, Campylobacter jejuni, Campylobacter fetus, Campylobacter coli, Chlamydia spp., such as Chlamydia pneumoniae and Chlamydia trachomatis, Citrobacter freundii, Citrobacter koseri, Coxiella burnetii, Edwarsiella spp., such as, Edwarsiella tarda, Eikenella corrodens, Enterobacter spp., such as, Enterobacter cloacae, Enterobacter aerogenes, and Enterobacter agglomerans, Escherichia coli, Francisella tularensis, Haemophilus influenzae, Haemophilus ducreyi, Helicobacter pylori, Kingella kingae, Klebsiella spp., such as, Klebsiella pneumoniae, Klebsiella oxytoca, Klebsiella rhinoscleromatis, and Klebsiella ozaenae, Legionella penumophila, Moraxella spp., such as, Moraxella catarrhalis, Morganella spp., such as, Morganella morganii, Neisseria gonorrhoeae, Neisseria meningitidis, P. aeruginosa, Pasteurella multocida, Plesiomonas shigelloides, Proteus mirabilis, Proteus vulgaris, Proteus penneri, Proteus myxofaciens, Providencia spp., such as, Providencia stuartii, Providencia rettgeri, Providencia alcalifaciens, Pseudomonas fluorescens, Salmonella typhi, Salmonella typhimurium, Salmonella paratyphi, Serratia spp., such as, Serratia marcescens, Shigella spp., such as, Shigella flexneri, Shigella boydii, Shigella sonnei, and Shigella dysenteriae, Stenotrophomonas maltophilia, Streptobacillus moniliformis, Vibrio cholerae, Vibrio parahaemolyticus, Vibrio vulnificus, Vibrio alginolyticus, Yersinia enterocolitica, Yersinia pestis, Yersinia pseudotuberculosis, Chlamydia pneumoniae, Chlamydia trachomatis, Ricketsia prowazekii, Coxiella burnetii, Ehrlichia chafeensis and/or Bartonella hensenae.

More typically, the at least one other species of Gram-negative bacteria is selected from one or more of Acinetobacter baumannii, Bordetella pertussis, Burkholderia cepacia, Burkholderia pseudomallei, Burkholderia mallei, Campylobacter jejuni, Campylobacter coli, Enterobacter cloacae, Enterobacter aerogenes, Escherichia coli, Francisella tularensis, Haemophilus influenzae, Haemophilus ducreyi, Helicobacter pylori, Klebsiella pneumoniae, Legionella penumophila, Moraxella catarrhalis, Morganella morganii, Neisseria gonorrhoeae, Neisseria meningitidis, Pasteurella multocida, Proteus mirabilis, Proteus vulgaris, Salmonella typhi, Serratia marcescens, Shigella flexneri, Shigella boydii, Shigella sonnei, Shigella dysenteriae, Stenotrophomonas maltophilia, Vibrio cholerae, and/or Chlamydia pneumoniae.

Even more typically, the at least one other species of Gram-negative bacteria is selected from one or more of Salmonella typhimurium, Salmonella typhi, Shigella spp., Escherichia coli, Acinetobacter baumanii, Klebsiella pneumonia, Neisseria gonorrhoeae, Neisseria meningitides, Serratia spp. Proteus mirabilis, Morganella morganii, Providencia spp., Edwardsiella spp., Yersinia spp., Haemophilus influenza, Bartonella quintana, Brucella spp., Bordetella pertussis, Burkholderia spp., Moraxella spp., Francisella tularensis, Legionella pneumophila, Coxiella burnetii, Bacteroides spp., Enterobacter spp., and/or Chlamydia spp.

Yet even more typically, the one or more additional species of Gram-negative bacteria are Klebsiella spp., Enterobacter spp., Escherichia coli, Citrobacter freundii, Salmonella typhimurium, Yersinia pestis, and/or Franciscella tulerensis.

In some embodiments, infection with Gram-negative bacteria results in a localized infection, such as a topical bacterial infection, e.g., a skin wound. In other embodiments, the bacterial infection is a systemic pathogenic bacterial infection. Common Gram-negative pathogens and associated infections are listed in Table 2 of the present disclosure. These are meant to serve as examples of the bacterial infections that may be treated, mitigated or prevented with the present lysins, active fragments thereof and lysin-AMP polypeptide constructs and are not intended to be limiting.

TABLE 2
Medically relevant Gram-negative bacteria and associated diseases.

| | |
|---|---|
| Salmonella typhimurium | Gastrointestinal (GI) infections- |
| | salmonellosis |
| Shigella spp. | shigellosis |
| Escherichia coli | Urinary tract infections (UTis) |
| Acinetobacter baumanii | Wound infections |
| Pseudomonas aeruginosa | bloodstream infections and pneumonia |
| Klebsiella pneumoniae | UTis, and bloodstream infections |
| Neisseria gonorrhoeae | Sexually transmitted disease (STD)- |
| | gonorrhea |
| Neisseria meningitides | Meningitis |
| Serratia spp. | Catheter contaminations, UTIs, and |
| | pneumonia |
| Proteus mirabilis | UTIs |
| Morganella spp. | UTIs |
| Providencia spp. | UTIs |
| Edwardsiella spp | UTIs |
| Salmonella typhi | GI infections - typhoid fever |
| Yersinia pestis | Bubonic and pneumonic plague |
| Yersinia enterocolitica | GI infections |
| Yersinia pseudotuberculosis | GI infections |
| Haemophilus influenza | Meningitis |
| Bartonella Quintana | Trench fever |
| Brucella spp. | Brucellosis |
| Bordetella pertussis | Respiratory - Whooping cough |
| Burkholderia spp. | Respiratory |
| Moraxella spp. | Respiratory |
| Francisella tularensis | Tularemia |
| Legionella pneumophila | Respiratory - Legionnaires' |
| | disease |
| Coxiella burnetiid | Q fever |
| Bacteroides spp. | Abdominal infections |
| Enterobacter spp. | UTis and respiratory |
| Chlamydia spp. | STDs, respiratory, and ocular |
| Escherichia coli, Klebsiella | Infections of implants, catheters, |
| pneumoniae, Acinetobacter | prosthetic joints and other medical |
| spp., Proteus mirabilis and/or | devices |
| Pseudomonas aeruginsa | |

In some embodiments, the lysin-AMP polypeptides, lysin polypeptides, variants, active fragments thereof or derivatives of the present disclosure are used to treat a subject at risk for acquiring an infection due to P. aeruginosa and/or another Gram-negative bacterium. Subjects at risk for acquiring a P. aeruginosa or other Gram-negative bacterial infection include, for example, cystic fibrosis patients, neutropenic patients, patients with necrotising enterocolitis, burn victims, patients with wound infections, and, more generally, patients in a hospital setting, in particular surgical patients and patients being treated using an implantable medical device such as a catheter, for example a central venous catheter, a Hickman device, or electrophysiologic cardiac devices, for example pacemakers and implantable defibrillators. Other patient groups at risk for infection with Gram-negative bacteria including P. aeruginosa include without limitation patients with implanted prostheses such a total joint replacement (for example total knee or hip replacement).

In another aspect, the present disclosure is directed to a method of preventing or treating a bacterial infection comprising co-administering to a subject diagnosed with, at risk for, or exhibiting symptoms of a bacterial infection, a combination of a first effective amount of the composition containing an effective amount of a lysin-AMP polypeptide, lysin polypeptide, variant, active fragment thereof or derivative as described herein, and a second effective amount of an antibiotic suitable for the treatment of Gram-negative bacterial infection.

The lysin-AMP polypeptides, lysin polypeptides, variants, active fragments thereof or derivatives of the present disclosure can be co-administered with standard care antibiotics or with antibiotics of last resort, individually or in various combinations as within the skill of the art. Traditional antibiotics used against P. aeruginosa are described in Table 3. Antibiotics for other Gram-negative bacteria, such as Klebsiella spp., Enterobacter spp., Escherichia coli, Citrobacter freundii, Salmonella typhimurium, Yersinia pestis, and Franciscella tulerensis, are similar to that provided in Table 3 for P. aeruginosa.

TABLE 3
Antibiotics used for the treatment of Pseudomonas aeruginosa

| Class | Agent |
|---|---|
| Penicillins | Ticarcillin-clavulanate |
| | Piperacillin-tazobactam |
| Cephalosporins | Ceftazidime |
| | Cefepime |
| | Cefoperazone |
| Monobactams | Aztreonam |
| Fluoroquinolones | Ciprofloxacin |
| | Levofloxacin |
| Carbapemens | Imipenem |
| | Meropenem |
| | Doripenem |
| Aminoglycosides | Gentamicin |
| | Tobramycin |
| | Amikacin |
| Polymixins | Colistin |
| | Polymixin B |
| Macrolides | Azithromycin |
| Rifamycin | Rifampicin |
| Fosfomycin | Fosfomycin |

In more specific embodiments, the antibiotic is selected from one or more of ceftazidime, cefepime, cefoperazone, ceftobiprole, ciprofloxacin, levofloxacin, aminoglycosides, imipenem, meropenem, doripenem, gentamicin, tobramycin, amikacin, piperacillin, ticarcillin, penicillin, rifampicin, polymyxin B and colistin. In certain embodiments, the antibiotic is meropenem.

Combining lysin-AMP polypeptides, lysin polypeptides, variants, active fragments thereof or derivatives of the present disclosure with antibiotics provides an efficacious antibacterial regimen. In some embodiments, co-administration of lysin-AMP polypeptides, lysin polypeptides, variants, active fragments thereof or derivatives of the present disclosure with one or more antibiotics may be carried out at reduced doses and amounts of either the lysin-AMP polypeptides, lysin polypeptides, variants, active fragments thereof or derivatives or the antibiotic or both, and/or reduced frequency and/or duration of treatment with augmented bactericidal and bacteriostatic activity, reduced risk of antibiotic resistance and with reduced risk of deleterious neurological or renal side effects (such as those associated with colistin or polymyxin B use). Prior studies have shown that total cumulative colistin dose is associated with kidney damage, suggesting that decrease in dosage or shortening of treatment duration using the combination therapy with lysin-AMP polypeptides, lysin polypeptides, variants, active fragments thereof or derivatives could decrease the incidence of nephrotoxicity (Spapen et al. Ann Intensive Care. 1: 14 (2011), which is herein incorporated by reference in its entirety). As used herein the term “reduced dose” refers to the dose of one active ingredient in the combination compared to monotherapy with the same active ingredient. In some embodiments, the dose of the lysins, active fragments thereof and lysin-AMP polypeptide constructs or the antibiotic in a combination may be suboptimal or even subthreshold compared to the respective monotherapy.

In some embodiments, the present disclosure provides a method of augmenting antibiotic activity of one or more antibiotics against Gram-negative bacteria compared to the activity of said antibiotics used alone by administering to a subject one or more lysin-AMP polypeptides, lysin polypeptides, variants, active fragments thereof or derivatives disclosed herein together with an antibiotic of interest. The combination is effective against the bacteria and permits resistance against the antibiotic to be overcome and/or the antibiotic to be employed at lower doses, decreasing undesirable side effects, such as the nephrotoxic and neurotoxic effects of polymyxin B.

The lysin-AMP polypeptides, lysin polypeptides, variants, active fragments thereof or derivatives optionally in combination with antibiotics of the present disclosure can be further combined with additional permeabilizing agents of the outer membrane of the Gram-negative bacteria, including, but not limited to metal chelators, such as e.g. EDTA, TRIS, lactic acid, lactoferrin, polymyxins, citric acid (Vaara M. Microbial Rev. 56(3):395-441 (1992), which is herein incorporated by reference in its entirety).

In yet another aspect, the present disclosure is directed to a method of inhibiting the growth, or reducing the population, or killing of at least one species of Gram-negative bacteria, the method comprising contacting the bacteria with a composition containing an effective amount of lysin-AMP polypeptides, lysin polypeptides, variants, active fragments thereof or derivatives as described herein, wherein the lysin-AMP polypeptides, lysin polypeptides, variants, active fragments thereof or derivatives inhibits the growth, or reduces the population, or kills P. aeruginosa and optionally at least one other species of Gram-negative bacteria.

In some embodiments, inhibiting the growth, or reducing the population, or killing at least one species of Gram-negative bacteria comprises contacting bacteria with the lysins, active fragments thereof and/or lysin-AMP polypeptide constructs as described herein, wherein the bacteria are present on a surface of e.g., medical devices, floors, stairs, walls and countertops in hospitals and other health related or public use buildings and surfaces of equipment in operating rooms, emergency rooms, hospital rooms, clinics, and bathrooms and the like.

Examples of medical devices that can be protected using the lysin-AMP polypeptides, lysin polypeptides, variants, active fragments thereof or derivatives described herein include but are not limited to tubing and other surface medical devices, such as urinary catheters, mucous extraction catheters, suction catheters, umbilical cannulae, contact lenses, intrauterine devices, intravaginal and intraintestinal devices, endotracheal tubes, bronchoscopes, dental prostheses and orthodontic devices, surgical instruments, dental instruments, tubings, dental water lines, fabrics, paper, indicator strips (e.g., paper indicator strips or plastic indicator strips), adhesives (e.g., hydrogel adhesives, hot-melt adhesives, or solvent-based adhesives), bandages, tissue dressings or healing devices and occlusive patches, and any other surface devices used in the medical field. The devices may include electrodes, external prostheses, fixation tapes, compression bandages, and monitors of various types. Medical devices can also include any device which can be placed at the insertion or implantation site such as the skin near the insertion or implantation site, and which can include at least one surface which is susceptible to colonization by Gram-negative bacteria.

The lysin-AMP polypeptides, lysin polypeptides, variants, active fragments thereof or derivatives of the present disclosure, which can be used in vivo or in vitro as described herein may also be used to treat bacterial infections due to Gram-negative bacteria, such as P. aeruginosa, that are associated with biofilm formation.

For example, in some embodiments, the present lysin-AMP polypeptides, lysin polypeptides, variants, active fragments thereof or derivatives may be used for the prevention, control, disruption, and/or eradication of bacterial biofilm formed by Gram-negative bacteria, such as P. aeruginosa. Biofilm formation occurs when microbial cells adhere to each other and are embedded in a matrix of extracellular polymeric substance (EPS) on a surface. The growth of microbes in such a protected environment that is enriched with biomacromolecules (e.g. polysaccharides, nucleic acids and proteins) and nutrients allows for enhanced microbial cross-talk and increased virulence. Biofilm may develop in any supporting environment including living and nonliving surfaces such as the mucus plugs of the CF lung, contaminated catheters, contact lenses, etc (Sharma et al. Biologicals, 42(1):1-7 (2014), which is herein incorporated by reference in its entirety). Thus, in one embodiment, the lysin-AMP polypeptides, lysin polypeptides, variants, active fragments thereof or derivatives of the present disclosure can be used for the prevention, control, disruption, eradication and treatment of bacterial infections due to Gram-negative bacteria, such as P. aeruginosa, when the bacteria are protected by a bacterial biofilm.

More particularly, in some aspects, the present disclosure is directed to a method for prevention, disruption or eradication of a Gram-negative bacterial biofilm comprising contacting a surface, including a biotic or abiotic surface, with a composition comprising a lysin-AMP polypeptide, lysin polypeptide, variant, active fragment thereof or derivative of the present disclosure effective to kill Gram negative bacteria, wherein a biofilm is effectively prevented, disrupted or eradicated.

In some aspects, the present disclosure is directed to a method for prevention, disruption or eradication of a Gram-negative bacterial biofilm comprising administering a composition to a subject in need thereof, wherein the composition comprises a lysin-AMP polypeptide, lysin polypeptide, variant, active fragment thereof or derivative of the present disclosure effective to kill Gram negative bacteria on a surface, wherein a biofilm is effectively prevented, disrupted or eradicated.

In some embodiments, the surface is a biotic surface, such as a solid biological surface, e.g., skin. In other embodiments, the surface is a non-biotic surface. In some embodiments, the surface is a surface of a medical device such as contact lenses; drug pumps, implants, including dental implants, cardiac implants such as pacemakers, prosthetic heart valves, ventricular assist devices, synthetic vascular grafts and stents; catheters including peritoneal dialysis catheters, indwelling catheters for hemodialysis and for chronic administration of chemotherapeutic agents (Hickman catheters), urinary catheters and prosthetic devices including urinary tract prostheses, prosthetic joints; orthopedic material; and tracheal and ventilator tubing.

In some embodiments, the subject is suffering from a Gram-negative bacterial infection associated with a biofilm. Such bacterial infections include tonsillitis, osteomyelitis, bacterial endocarditis, sinusitis, infections of the cornea, urinary tract infection, infection of the biliary tract, infectious kidney stones, urethritis, prostatitis, middle-ear infections, formation of dental plaque, gingivitis, periodontitis, cystic fibrosis, wound infections, in particular wounds associated with diabetes mellitus, and infections of medical devices as described herein including catheter infections and infections of joint prostheses and heart valves.

In some embodiments, the composition for treating biofilm infections comprises one or more antibiotics as described herein. In other embodiments, the present lysins or active fragments thereof or variants or derivatives thereof as described herein are administered to a subject and/or contacted to a surface simultaneously with one or more antibiotics as herein described. In other embodiments, a lysin-AMP polypeptide, lysin polypeptide, variant, active fragment thereof or derivative of the present disclosure and the one or more antibiotics as described herein are administered to a subject and/or contacted to a surface sequentially in any order. In some embodiments, the present lysin-AMP polypeptides, lysin polypeptides, variants, active fragments thereof or derivatives of the present disclosure and the one or more antibiotics as described herein may be administered to a subject and/or contacted to a surface in a single dose or multiple doses, singly or in combination.

In some embodiments, the present composition is used to prevent biofilm formation. In these embodiments, the contacted surface may contain a biofilm, may not contain a biofilm, or contains only de minimus amounts of an established biofilm. In some embodiments, de novo biofilm formation on the surface is prevented according to any mechanisms as described herein.

In some embodiments, the contacted surface comprises a biofilm and the biofilm is disrupted or eradicated. In some embodiments, eradication comprises killing bacteria in the biofilm, including persister bacteria. In other embodiments, the present lysin-AMP polypeptides, lysin polypeptides, variants, active fragments thereof or derivatives of the present disclosure not only kill bacteria within a biofilm, thus eradicating the biofilm, but also disrupt or destroy the biofilm matrix. This ability is advantageous since matrices, even in the absence of live bacteria, often become quickly re-infected.

### EXAMPLES

### Example 1. Activity of Lysins and Lysin-AMP Polypeptide Constructs in Medium Supplemented with Human Serum

Materials and Methods

Gram-negative bacteria, e.g., P. aeruginosa, were cultured and tested in casamino acid (CAA) media (5 g/L casamino acids, Ameresco/VWR; 5.2 mM K2HPO4, Sigma-Aldrich; 1 mM MgSO4, Sigma-Aldrich), CAA supplemented with 150 mM NaCl, CAA supplemented with 2.5% human serum (Type AB, male, pooled; Sigma-Aldrich), CAA supplemented with 12.5% human serum, and CAA supplemented with 6.25% Survanta®. For both the CAA supplemented with 12.% human serum and 6.25% Survanta®, a range of P. aeruginosa isolates were evaluated. 6.25% Survanta® corresponds to 1.5 mg/mL phospholipids.

Determination of Minimal Inhibitory Concentrations (MIC)

MIC values were determined using a modification of the standard broth microdilution reference method defined by the Clinical and Laboratory Standards Institute (CLSI), CLSI. 2015. Methods for Dilution Antimicrobial Susceptibility Tests for Bacteria That Grow Aerobically; Approved Standard-10th Edition. Clinical and Laboratory Standards Institute, Wayne, Pa. The modification was based on the replacement of Mueller Hinton Broth with either CAA media (with and without NaCl), CAA supplemented with 2.5% human serum (Table 4), CAA supplemented with 12.5% human serum (Table A), or CAA supplemented with 6.25% Survanta® (Table B). MIC is the minimum concentration of peptide sufficient to suppress at least 80% of the bacterial growth compared to control.

Results

The results of these experiments are summarized in Tables 4, A and B below. Table 4 also provides the molecular weight and isoelectric point of the present polypeptides. By comparing the sequences and components of the various polypeptides, the effect of a particular structural modification on isoelectric point (a higher pI favors outer membrane penetration) and activity (as assessed by MIC) can be determined.

For example, the effects of the single point mutations on GN316 (SEQ ID NO: 22) can be seen. GN394 (SEQ ID NO: 48) has a lower pI and a higher activity in CAA but a lower activity in CAA with human serum. The activity reduction in human serum is less for GN396 (SEQ ID NO: 50), whereas GN408 (SEQ ID NO: 52) is substantially more potent both in the presence and in the absence of human serum. On the other hand GN418 (SEQ ID NO: 54) loses activity in unsupplemented CAA media but gains potency in the presence of human serum.

The single point mutation in GN217 (SEQ ID NO: 8) improves its potency over GN37 both in the absence and presence of human serum. The modifications to GN37 (SEQ ID NO: 84) yielding GN218 (SEQ ID NO: 10), GN223 (SEQ ID NO: 12), GN239 (SEQ ID NO: 14) and GN243 (SEQ ID NO: 16) result in very strong activity in the presence of human serum. Similar observations can be made based on comparison of the sequence and components of other polypeptides.

### Example 2. Synergy Between Antibiotics and Lysins or Lysin-AMP Polypeptide Constructs

Synergy between GN76 (SEQ ID NO: 203), GN121 (SEQ ID NO: 175), GN123 (SEQ ID NO: 173), GN351 (SEQ ID NO: 32), GN370 (SEQ ID NO: 44) and GN428 (SEQ ID NO: 60) and 12 different antibiotics were examined in checkerboard assays using CAA medium, supplemented with human serum as described herein, using the carbapenem-resistant clinical strain WC-452. Fractional inhibitor concentration index (FICI) values were determined for all combinations; values of <0.5 indicate synergy.

As indicated in Table 5, below, the foregoing lysins and lysin-AMP constructs are synergistic across a broad range of antibiotics. For imipenem, the synergy is consistent with resensitization to the carbapenem antibiotic.

### Example 3. Resensitization of Carbapenem-Resistant Clinical Strains Using Antibiotics in Combination with Lysins

The ability of GN121 (SEQ ID NO: 175) or GN123 (SEQ ID NO: 173) to resensitize carbapenem-resistant P. aeruginosa strains to carbapenems was assessed by combining each of the foregoing lysins with two carbapenems, i.e., imipenem (IPM) or meropenem (MEM). Up to seven carbapenem-resistant isolates were assessed. Resensitization occurs in synergistic combinations in which the carbapenem MIC values fall below established breakpoints, e.g. a MIC value of ≤2 for carbapenem-sensitive isolates, a MIC value of 4 for intermediately sensitive carbapenem isolates and a MIC value of ≥8 for carbapenem-resistant isolates. See Clinical and Laboratory Standards Institute (CLSI), CLSI. 2019. M100 Performance Standards for Antimicrobial Susceptibility Testing; 29th Edition. Clinical and Laboratory Standards Institute, Wayne, Pa.

As indicated in Tables 6-9 synergistic combinations with GN123 (SEQ ID NO: 173) or GN121 (SEQ ID NO: 175) demonstrated reductions of IPM and MEM MICS to below breakpoint values for each of the seven carbapenems examined. These observations are consistent with resensitization.

### Example 4. Resensitization of Carbapenem-Resistant Clinical Strains Using Antibiotics in Combination with Additional Lysins or Lysin-AMP Constructs

The ability of GN351 (SEQ ID NO: 32), GN370 (SEQ ID NO: 44) or GN428 (SEQ ID NO: 60) to resensitize carbapenem-resistant clinical strains to carbapenems was assessed by combining each of the foregoing lysins or lysin-AMP polypeptide constructs with IPM or MEM. WC-452, a carbapenem-resistant isolate, was assessed. As noted in Example 3, above, resensitization occurs in synergistic combinations in which the carbapenem MIC values fall below the previously described breakpoints.

As indicated in Table 10 synergistic combinations with GN351 (SEQ ID NO: 32), GN370 (SEQ ID NO: 44) or GN428 (SEQ ID NO: 60) demonstrated reductions of IPM and MEM MICS to below breakpoint values for WC-452. These observations are consistent with resensitization.

The findings in Examples 3 and 4 indicate that the lysins and lysin-AMP polypeptide constructs described herein can resensitize P. aeruginosa to carbapenem antibiotics, driving MICs below breakpoint values in vitro. This novel ability of lysins and lysin-AMP polypeptide constructs to resensitize antibiotic-resistant strains to conventional antibiotics indicates the benefit of these biologics as therapeutics to combat and reverse antimicrobial resistance.

TABLE A
Activity of lysins or lysin-AMP polypeptide
constructs in human serum

| | Meropenem | CAA + 12.5% Human | | | |
|---|---|---|---|---|---|
| P. aeruginosa | MIC | Serum MIC (μg/mL) | | | |
| Strain | (μg/mL) | GN121 | GN351 | GN370 | GN428 |
| CFS 1292 | 32 | 1 | 1 | 2 | 2 |
| CFS 1557 (PA19) | 32 | 2 | 4 | 4 | 4 |
| CFS 1558 (PA20) | 16 | 0.5 | 1 | 0.5 | 2 |
| CFS 1559 (PA21) | >32 | 1 | 2 | 2 | 2 |
| CFS 1560 (PA22) | 16 | 1 | 2 | 2 | 2 |
| CFS 1561 (PA23) | 16 | 1 | 2 | 2 | 2 |
| CFS 1562 (PA24) | >32 | 1 | 2 | 2 | 2 |
| CFS 1766 (ATCC | 1 | 2 | 2 | 4 | 4 |
| 27853) | | | | | |
| CFS 1539 (PA1) | 16 | 0.5 | 0.5 | 1 | 1 |
| CFS 1540 (PA2) | 16 | 0.5 | 0.5 | 1 | 1 |
| CFS 1541 (PA3) | 8 | 0.5 | 0.5 | 1 | 1 |
| CFS 1596 (PA26) | 0.5 | 0.5 | 1 | 1 | 1 |
| CFS 1597 (PA27) | 1 | 0.5 | 0.5 | 0.5 | 0.5 |
| CFS 1669 (PA41) | <0.25 | 1 | 1 | 2 | 2 |
| CFS 1674 (PA46) | 4 | 0.5 | 1 | 2 | 2 |
| CFS 1675 (PA47) | 4 | 0.5 | 0.5 | 1 | 1 |
| CFS 1109 (ATCC | 0.5 | 0.5 | 1 | 1 | 1 |
| 17646) | | | | | |

TABLE B
Activity in pulmonary surfactant (Survanta ®)

| | |
|---|---|
| | Fold change in MIC for |
| P. aeruginosa | CAA + 6.25% Human Serum |

| Strain | GN121 | GN351 | GN370 | GN428 |
|---|---|---|---|---|
| CFS 1292 | 1 | 2 | 1 | 1 |
| CFS 1557 (PA19) | 2 | 1 | 0.5 | 0.5 |
| CFS 1558 (PA20) | 2 | 2 | 1 | 1 |
| CFS 1559 (PA21) | 2 | 2 | 1 | 1 |
| CFS 1560 (PA22) | 2 | 2 | 1 | 1 |
| CFS 1561 (PA23) | 1 | 1 | 1 | 1 |
| CFS 1562 (PA24) | 2 | 1 | 0.5 | 1 |
| CFS 1766 (ATCC | 1 | 1 | 1 | 2 |
| 27853) | | | | |
| CFS 1539 (PA1) | 1 | 1 | 0.5 | 0.5 |
| CFS 1540 (PA2) | 1 | 1 | 1 | 1 |
| CFS 1541 (PA3) | 2 | 2 | 1 | 1 |
| CFS 1596 (PA26) | 2 | 2 | 1 | 1 |
| CFS 1597 (PA27) | 2 | 1 | 0.5 | 0.5 |
| CFS 1669 (PA41) | 2 | 0.5 | 0.5 | 0.5 |
| CFS 1674 (PA46) | 2 | 2 | 0.5 | 1 |
| CFS 1675 (PA47) | 1 | 0.5 | 0.5 | 0.5 |
| CFS 1109 (ATCC | 2 | 1 | 1 | 1 |
| 17646) | | | | |

TABLE 4
Sensitivity of lysins or lysin-AMP
polypeptide constructs in human serum
MIC (mg/mL)

| | | | CAA | CAA/ |
|---|---|---|---|---|
| | | | MIC | HuS MIC |
| GN # | MW | pI | (mg/mL) | (mg/mL) |
| GN168 (SEQ ID NO: 2) | 22299.78 | 11.6 | 8 | N.D. |
| GN176 (SEQ ID NO: 4) | 19370 | 9.8 | 8 | N.D. |
| GN178 (SEQ ID NO: 6) | 19290.04 | 9.7 | 8 | 4 |
| GN217 (SEQ ID NO: 8) | 13879.91 | 9.4 | 4 | 0.125 |
| GN218 (SEQ ID NO: 10) | 16038.43 | 9.8 | 8 | 1 |
| GN223 (SEQ ID NO: 12) | 18570.35 | 10.3 | 32 | 2 |
| GN239 (SEQ ID NO: 14) | 16836.42 | 10.2 | 4 | 0.25 |
| GN243 (SEQ ID NO; 16) | 18880.02 | 10.5 | 32 | 0.5 |
| GN280 (SEQ ID NO: 18) | 17928.9 | 10.2 | 4 | 0.5 |
| GN281 (SEQ ID NO: 20) | 18188.07 | 10.2 | 2 | 0.5 |
| GN316 (SEQ ID NO: 22) | 28672.72 | 8.7 | 16 | 0.125 |
| GN329 (SEQ ID NO: 26) | 20810.83 | 8.9 | 4 | 0.25 |
| GN333 (SEQ ID NO: 28) | 20918.79 | 8.9 | 8 | 0.06 |
| GN349 (SEQ ID NO: 30) | 34169.19 | 9.5 | 16 | 1 |
| GN351 (SEQ ID NO: 32) | 33866.76 | 9.9 | 8 | 0.125 |
| GN352 (SEQ ID NO: 34) | 33398.27 | 8.9 | 4 | 0.5 |
| GN353 (SEQ ID NO: 36) | 33485.42 | 8.9 | 4 | 0.25 |
| GN357 (SEQ ID NO: 38) | 30891.39 | 9.3 | 16 | 0.25 |
| GN359 (SEQ ID NO: 40) | 31094.67 | 8.7 | 8 | 0.25 |
| GN369 (SEQ ID NO: 42) | 30934.63 | 8.8 | 8 | 0.0625 |
| GN370 (SEQ ID NO: 44) | 19140.86 | 10.7 | 16 | 4 |
| GN371 (SEQ ID NO: 46) | 17530.95 | 8.7 | >32 | 0.5 |
| GN394 (SEQ ID NO: 48) | 28659.62 | 7.5 | 8 | 0.5 |
| GN396 (SEQ ID NO: 50) | 28659.62 | 7.5 | 8 | 0.25 |
| GN408 (SEQ ID NO: 52) | 28653.66 | 7.8 | 2 | 0.125 |
| GN418 (SEQ ID NO: 54) | 28659.62 | 7.5 | 32 | 0.06 |
| GN424 (SEQ ID NO: 56) | 29118.75 | 8.4 | ND | ND |
| GN425 (SEQ ID NO: 58) | 29895.81 | 7.5 | 2 | 0.25 |
| GN428 (SEQ ID NO: 60) | 28814.89 | 8.9 | 8 | 0.125 |
| GN93 (SEQ ID NO: 62) | 22959.07 | 9.6 | 128 | 8 |
| GN431 (SEQ ID NO: 64) | 28715.73 | 8.5 | 8 | 0.0625 |
| GN486 (SEQ ID NO: 66) | 17.8 | 10.6 | 2 | 0.125 |
| GN485 (SEQ ID NO: 68) | 8.312 | 9.8 | n.d. | n.d. |

TABLE 5
Synergy between antibiotics and lysins or lysin-AMP polypeptide constructs

| | GN76 | GN121 | GN123 | GN351 | GN370 | GN428 |
|---|---|---|---|---|---|---|
| | (SEQ ID | (SEQ ID | (SEQ ID | (SEQ ID | (SEQ ID | (SEQ ID |
| | NO: 203) | NO: 175) | NO: 173) | NO: 32) | NO: 44) | NO: 60) |
| Antibiotic | (MIC) | (MIC) | (MIC) | (MIC) | (MIC) | (MIC) |
| Amikacin | 0.281 | 0.375 | 0.250 | 0.250 | 0.125 | 0.281 |
| Azithromycin | 0.156 | 0.188 | 0.125 | 0.125 | 0.188 | 0.250 |
| Aztreonam | 0.281 | 0.625 | 0.375 | 0.125 | 0.188 | 0.156 |
| Ciprofloxacin | 0.281 | 0.313 | 0.375 | 0.375 | 0.281 | 0.125 |
| Collstin | 0.250 | 0.046 | 0.188 | 0.046 | 0.046 | 0.094 |
| Fosfomycin | 0.125 | 0.375 | 0.250 | 0.500 | 0.375 | 0.313 |
| Gentamicin | 0.313 | 0.375 | 0.375 | 0.125 | 0.250 | 0.250 |
| Imipenem | 0.254 | 0.375 | 0.188 | 0.156 | 0.094 | 0.188 |
| Meropenem | 0.375 | 0.313 | 0.125 | 0.188 | 0.125 | 0.188 |
| Pipercillan | 0.375 | 0.375 | 0.500 | 0.281 | 0.125 | 0.375 |
| Rifampicin | 0.281 | 0.313 | 0.156 | 0.250 | 0.250 | 0.500 |
| Tobramycin | 0.281 | 0.188 | 0.188 | 0.153 | 0.188 | 0.188 |

TABLE 6
Gram-negative bacterial resensitization using a combination
of IMIPENEM and GN123 (SEQ ID NO: 173)

| | IMIPENEM MIC (μg/mL) | | | | GN123 (μg/mL) | | |
|---|---|---|---|---|---|---|---|
| Isolate | Alone | | Combination | | Alone | Combination | FICI |
| PA19 | 32 | (R) | 0.5 | (S) | 8 | 0.125 | 0.03 |

Analysis of additional CARBAPENEM^{R} isolates:

| | | | | | | | |
|---|---|---|---|---|---|---|---|
| PA20 | 16 | (R) | 1 | (S) | 16 | 2 | 0.188 |
| PA21 | 32 | (R) | 0.5 | (S) | 8 | 1 | 0.141 |
| PA22 | 16 | (R) | 2 | (S) | 16 | 1 | 0.188 |
| PA23 | 8 | (R) | 0.25 | (S) | 8 | 2 | 0.281 |
| PA24 | 32 | (R) | 2 | (S) | 16 | 2 | 0.188 |
| WC-452 | 16 | (R) | 1 | (S) | 16 | 2 | 0.188 |

(R) = resistant
(S) = sensitive

TABLE 7
Gram-negative bacterial resensitization using a combination
of MEROPENEM and GN123 (SEQ ID NO: 173)

| | MEROPENEM MIC (μg/mL) | | | GN123 (μg/mL) | | |
|---|---|---|---|---|---|---|
| Isolate | Alone | Combination | | Alone | Combination | FICI |
| PA19 | 32 (R) | 0.5 | (S) | 8 | 0.25 | 0.046 |
| PA20 | 16 (R) | 0.5 | (S) | 16 | 1 | 0.094 |
| PA21 | 32 (R) | 1 | (S) | 8 | 1 | 0.156 |
| PA22 | 16 (R) | 1 | (S) | 16 | 1 | 0.125 |
| PA23 | 16 (R) | 0.5 | (S) | 8 | 1 | 0.156 |
| PA24 | 32 (R) | 2 | (S) | 16 | 0.5 | 0.094 |
| WC-452 | 16 (R) | 1 | (S) | 16 | 1 | 0.125 |

(R) = resistant
(S) = sensitive

TABLE 8
Gram-negative bacterial resensitization using a combination
of IMIPENEM and GN121 (SEQ ID NO: 175)

| | Imipenem MIC (μg/mL) | | | GN121 (μg/mL) | | |
|---|---|---|---|---|---|---|
| Isolate | Alone | Combination | | Alone | Combination | FICI |
| PA19 | 32 (R) | 1 | (S) | 1 | 0.125 | 0.155 |
| PA20 | 16 (R) | 0.5 | (S) | 1 | 0.25 | 0.265 |
| PA21 | 32 (R) | 1 | (S) | 1 | 0.125 | 0.155 |
| PA22 | 32 (R) | 2 | (S) | 2 | 0.25 | 0.188 |
| PA23 | 16 (R) | 0.125 | (S) | 1 | 0.25 | 0.257 |
| PA24 | 32 (R) | 1 | (S) | 1 | 0.125 | 0.155 |

(R) = resistant
(S) = sensitive

TABLE 9
Gram-negative bacterial resensitization using a combination
of MEROPENEM and GN121 (SEQ ID NO: 175)

| | Meropenem MIC (μg/mL) | | GN121 (μg/mL) | | |
|---|---|---|---|---|---|
| Isolate | Alone | Combination | Alone | Combination | FICI |
| PA19 | 32 (R) | 1 | 2 | 0.5 | 0.281 |
| PA20 | 16 (R) | 1 | 2 | 0.5 | 0.313 |
| PA21 | 32 (R) | 2 | 1 | 0.125 | 0.188 |
| PA22 | 16 (R) | 1 | 1 | 0.25 | 0.313 |
| PA23 | 16 (R) | 2 | 2 | 0.5 | 0.375 |
| PA24 | 32 (R) | 1 | 1 | 0.125 | 0.156 |
| WC-452 | 16 (R) | 1 | 1 | 0.06 | 0.123 |

(R) = resistant;
(S) = sensitive

TABLE 10
Gram-negative bacterial resensitization using combinations
of MEM or IPM and GN351 (SEQ ID NO: 32), GN370
(SEQ ID NO: 44), or GN428 (SEQ ID NO: 60)

| Combinations | Antibiotic MIC | | | Lysin MIC | | |
|---|---|---|---|---|---|---|
| vs. WC-452 | Alone | Combination | | Alone | Combination | FICI |
| IPM + GN351 | 16 (R) | 0.5 | (S) | 1 | 0.125 | 0.156 |
| IPM + GN370 | 16 (R) | 0.5 | (S) | 2 | 0.125 | 0.094 |
| IPM + GN428 | 16 (R) | 1 | (S) | 2 | 0.25 | 0.188 |
| MEM + GN351 | 16 (R) | 1 | (S) | 1 | 0.125 | 0.188 |
| MEM + GN370 | 16 (R) | 0.5 | (S) | 2 | 0.125 | 0.125 |
| MEM + GN428 | 16 (R) | 1 | (S) | 2 | 0.25 | 0.188 |

### Example 5. Gram-Negative Lysin Bactericidal Activity Against Pseudomonas aeruginosa in Human Serum and Pulmonary Surfactant

Further characterization of the bacteriolytic activities of four anti-pseudomonal lysins described herein, GN121, GN351, GN370, and GN428, was evaluated using standard in vitro susceptibility testing formats that incorporate human serum or pulmonary surfactant. The mechanism of gram-negative lysin action was further evaluated by fluorescence and transmission electron microscopy (TEM), as discussed in Examples 6 and 7.

Materials and methods: MICs were determined by broth microdilution in media supplemented with human serum and pulmonary surfactant (Survanta®; Myoderm Clinical Supplies). Synergy with antibiotics was examined in checkerboard assays and minimal biofilm eradicating concentrations (MBECs) were determined using standard methods. MBEC was measured using CAA supplemented with 12.5% human serum. Fluorescence microscopy was performed after LIVE/DEAD staining (ThermoFisher) and TEM was performed.

Results: The activity of gram-negative lysins in human serum and pulmonary surfactant (Survanta®) was observed. Lysin MIC values were determined in the standard AST format medium (25% Casamino Acid Medium with 0.25 mM MgSO4) alone and in the presence of 12.5% human serum and 0.78% Survanta®. The Survanta® concentration of 0.78% represents a physiological level of pulmonary surfactant. Pseudomonas aeruginosa strain CFS-1292 (meropenem resistant) was used as the reporter strain. As shown in Table 11 below, it was concluded that the gram-negative lysins GN121, GN351, GN428, and GN370 are active in human serum and pulmonary surfactant. Likewise, as shown in Table C below, the lysins and AMP-lysin polypeptide constructs exhibited a potent antibiofilm effect using 12.5% human serum, with MBECs values <1 μg/mL, similar to those observed for MICs.

TABLE 11
MIC values for lysins in media alone (25% CAA) and supplemented
with human serum or pulmonary surfactant

| | | | MIC in human | |
|---|---|---|---|---|
| | | | serum (12.5%) | MIC in 0.78% |
| | Gram-negative | 25% | in CAA | Survanta ® |
| Clone | lysin | CAA MIC | (μg/mL) | (μg/mL) |
| 1525 | GN121 | 1 | 0.5 | 2 |
| 1799 | GN351 | 1 | 0.0625 | 4 |
| 1876 | GN428 | 4 | 0.125 | 4 |
| 1818 | GN370 | 4 | 2 | 2 |

TABLE C
MBEC values for lysins and lysin-AMP polypeptide constructs

| Lysin or Lysin-AMP | MBEC (μg/mL) in CAA supplemented |
|---|---|
| polypeptide construct | with 12.5% human serum |
| GN121 | 0.25 |
| GN351 | 0.5 |
| GN428 | 1 |
| GN370 | 1 |

The activity of gram-negative lysins in the presence of pulmonary surfactant (Survanta®) was measured over a range of concentrations of Survanta® in the MIC assay format. Fold changes in MIC in the presence of various concentrations of Survanta® (25%, 12.5%, 6.25%, 3.12%, 1.56%, 0.78%, and 0.39%) supplemented into the AST-format (25% SAA) is shown in Table 12. Fold changes are based on comparisons of MIC values to that determined in 25% CAA alone. Physiological concentrations of pulmonary surfactant range between 0.78% and 0.39%. Pseudomonas aeruginosa strain CFS-1292 (meropenem resistant) was used as the reporter strain.

It was concluded that the gram-negative lysins tested are active in the presence of physiological levels of pulmonary surfactant (Survanta®).

TABLE 12
Fold increase (MIC) in the presence of Survanta ®

| Gram- | | | | | | | | |
|---|---|---|---|---|---|---|---|---|
| negative | CAA MIC | % Survanta ® | | | | | | |
| lysin | (μg/mL) | 25 | 12.5 | 6.25 | 3.12 | 1.56 | 0.78 | 0.39 |
| GN121 | 2 | 4 | 2 | 2 | 2 | 1 | 1 | 1 |
| GN351 | 2 | 2 | 2 | 2 | 1 | 1 | 1 | 1 |
| GN428 | 4 | 4 | 2 | 1 | 1 | 1 | 1 | 1 |
| GN370 | 4 | 4 | 2 | 2 | 1 | 1 | 1 | 1 |

The activity of gram-negative lysins in the presence of divalent cations was evaluated, and the impact of divalent cations at physiological concentrations was examined in the MIC assay format. Fold changes in MIC were measured in the presence of various cation concentrations (1.25 mM CaCl₂, 0.78 mM MgCl₂, and a combination of 1.25 mM CaCl₂ and 0.78 mM MgCl₂) supplemented into the AST medium (25% CAA). The results are shown below in Table 13. It is noted that 25% CAA typically has 0.25 nM MgSO4. Pseudomonas aeruginosa strain CFS-1292 (meropenem resistant) was used as the reporter strain. It was concluded that the gram-negative lysins tested are active in the presence of physiological levels of calcium and magnesium.

TABLE 13
Fold Increase (MIC) in presence of cations

| | | 25% CAA supplemented with: | | |
|---|---|---|---|---|
| Gram- | | | | 1.25 mM |
| negative | 25% | 1.25 mM | 0.78 mM | CaCl₂ and 0.78 |
| lysin | CAA | CaCl₂ | MgCl₂ | mM MgCl₂ |
| GN121 | 1 | 2 | 2 | 2 |
| GN351 | 1 | 2 | 1 | 2 |
| GN428 | 4 | 2 | 4 | 4 |
| GN370 | 4 | 4 | 2 | 4 |

### Example 6. Ability of Gram-Negative Lysins to Destabilize Bacterial Outer Membrane

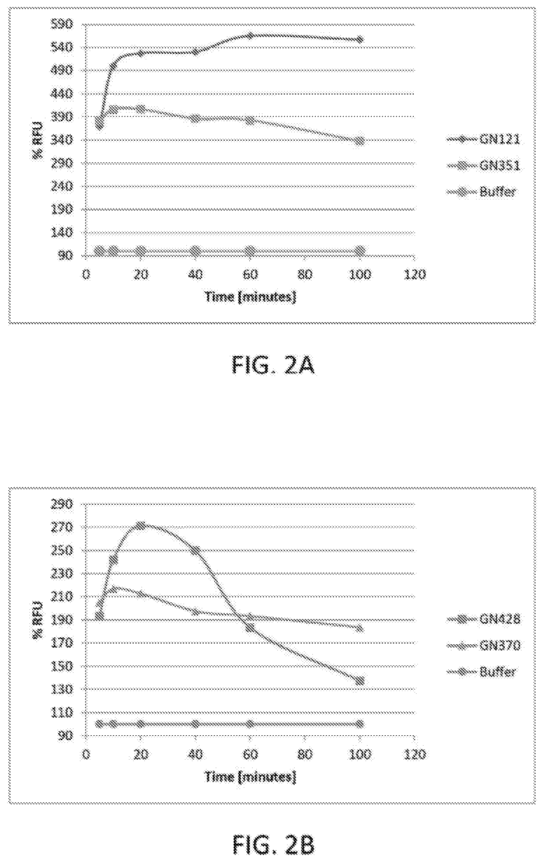

The ability of gram-negative lysins to destabilize the outer membrane of P. aeruginosa was evaluated through the use of an N-phenyl-1-napthylamine (NPN) uptake assay. See Dassanayake, R. P. et al., Antimicrobial activity of bovine NK-lysin-derived peptides on Mycoplasma bovis, PLOS One 2018; 9(1):e86364. Exponential P. aeruginosa (CFS 1292) was harvested, washed, and re-suspended in 5 mM 4-(2-hydroxyethyl)-1-piperazineethanesulfonic acid (HEPES) buffer and 5 mM glucose at pH 7.4. NPN was added to a final concentration of 10 mM. Gram-negative lysins, including GN121, GN351, GN428, and GN370, were added at a final concentration of 100 μg/well. Changes in fluorescence were recorded (excitation 1=350 nm; emission 1=420 nm) over two hours. The NPN incorporated into the membrane resulted in an increase in fluorescence. As shown in FIGS. 2A and 2B, the gram-negative lysins mediated disruption of the outer membrane of the bacterial cell wall. The data for each gram-negative lysin is shown below in Table 14.

TABLE 14
Fluorescence over time for P. aeruginosa
exposed to NPN and gram-negative lysins

| | |
|---|---|
| Time in | % RFU |

| minutes | Buffer | GN121 | GN351 | GN428 | GN370 |
|---|---|---|---|---|---|
| 5 | 100 | 370 | 381 | 194 | 205 |
| 10 | 100 | 500 | 406 | 242 | 217 |
| 20 | 100 | 528 | 407 | 271 | 213 |
| 40 | 100 | 530 | 386 | 250 | 198 |
| 60 | 100 | 565 | 383 | 183 | 193 |
| 100 | 100 | 557 | 338 | 137 | 184 |

### Example 7. Microscopy Shows Gram-Negative Lysin Bactericidality in Serum

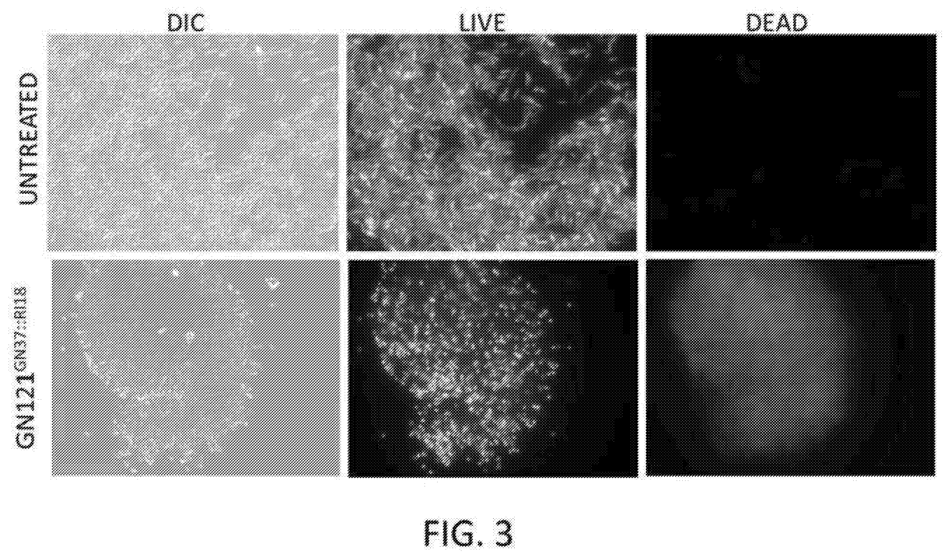

Pseudomonas aeruginosa strain 1292 was treated for 15 minutes with GN121 (10 μg/mL) or a buffer control in 100% human serum. Samples were stained using the Live/Dead Cell Viability Kit (ThermoFisher) and examined by both differential interference contrast (DIC) and fluorescence microscopy. As depicted in FIG. 3, which shows a series of photomicrographs showing microscopic analysis (×2000 magnification), there was an absence of dead bacteria in the untreated row and a reduction of live bacteria in the treated row.

### Example 8. Synergy of Gram-Negative Lysins and Meropenem in Human Serum

Standard checkerboard assays were performed to assess synergy of gram-negative lysins with meropenem in the presence of human serum. P. aeruginosa strains CFS 1292, 1557 (PA19), 1558 (PA20) CFS 1559 (PA21), CFS 1560 (PA22), CFS 1561 (PA23), CFS 1562 (PA24), and CFS 1766 (ATCC 27853) were suspended in a solution of 25% CAA and 12.5% human serum, and synergy was evaluated by measuring the fractional inhibitory concentration index (FICI) values. FICI values less than or equal to 0.5 were consistent with potent synergy. As shown below in Table 15, all of GN121, GN351, GN370, and GN428 exhibited synergy with meropenem for each of the three P. aeruginosa strains evaluated.

TABLE 15
Synergy between meropenem and gram-
negative lysins in human serum

| | Gram-negative | FICI value | FICI value |
|---|---|---|---|
| Strain | lysin | (Run #1) | (Run #2) |
| CFS 1292 | GN121 | 0.25 | 0.292 |
| | GN351 | 0.1875 | 0.219 |
| | GN370 | 0.1875 | 0.219 |
| | GN428 | 0.1875 | 0.219 |
| CFS 1557 | GN121 | 0.375 | 0.427 |
| (PA19) | GN351 | 0.25 | 0.292 |
| | GN370 | 0.1875 | 0.240 |
| | GN428 | 0.15625 | 0.198 |
| CFS 1558 | GN121 | 0.125 | 0.156 |
| (PA20) | GN351 | 0.15625 | 0.177 |
| | GN370 | 0.09375 | 0.109 |
| | GN428 | 0.09375 | 0.135 |
| CFS 1559 | GN121 | — | 0.229 |
| (PA21) | GN351 | — | 0.177 |
| | GN370 | — | 0.438 |
| | GN428 | — | 0.396 |
| CFS 1560 | GN121 | — | 0.313 |
| (PA22) | GN351 | — | 0.323 |
| | GN370 | — | 0.198 |
| | GN428 | — | 0.229 |
| CFS 1561 | GN121 | — | 0.198 |
| (PA23) | GN351 | — | 0.240 |
| | GN370 | — | 0.240 |
| | GN428 | — | 0.323 |
| CFS 1562 | GN121 | — | 0.214 |
| (PA24 | GN351 | — | 0.177 |
| | GN370 | — | 0.240 |
| | GN428 | — | 0.198 |
| CFS 1766 | GN121 | — | 0.229 |
| (ATCC 27853) | GN351 | — | 0.109 |
| | GN370 | — | 0.156 |
| | GN428 | — | 0.156 |

### Example 9. Low Propensity for Resistance to GN Lysins

In another experiment, it was determined that Gram-negative bacteria did not develop resistance to GN121, GN351, GN370, and GN428 in a 21-day serial passage resistance assay. An analysis of bacterial resistance was performed using P. aeruginosa (strain WC-452) over 21 days of serial passage in the presence of a GN-lysin dilution series (in duplicate). Briefly, the broth microdilution MIC format was used in which 2-fold dilution ranges of GN lysin were cultured with the bacteria 5×10e6 CFU/ml starting concentration) in CAA broth for 18 hours at 37° C. The well with the highest concentration of GN lysin in which bacterial growth was seen was then used as the inoculum for the next day's passage, and the process was repeated over a 21 day period. The MIC at each daily time-point was recorded, and resistance was measured as a step-wise increase in MIC.

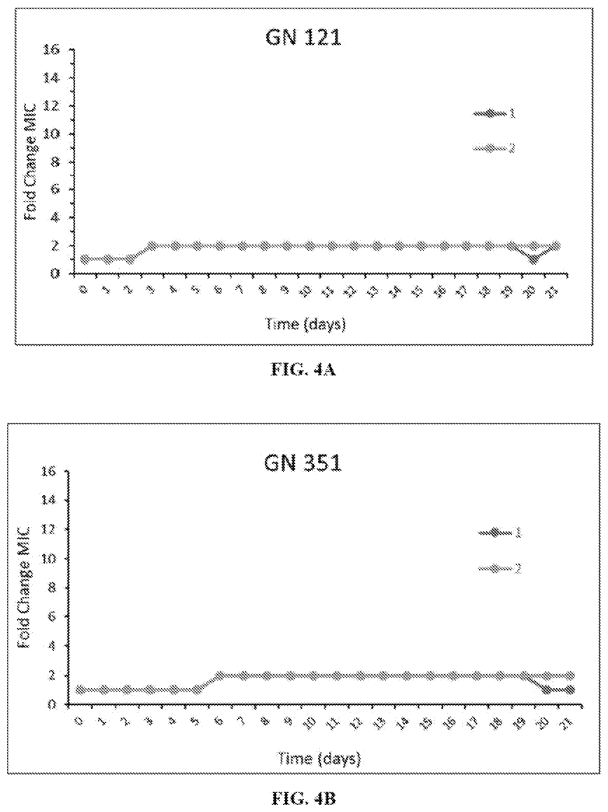

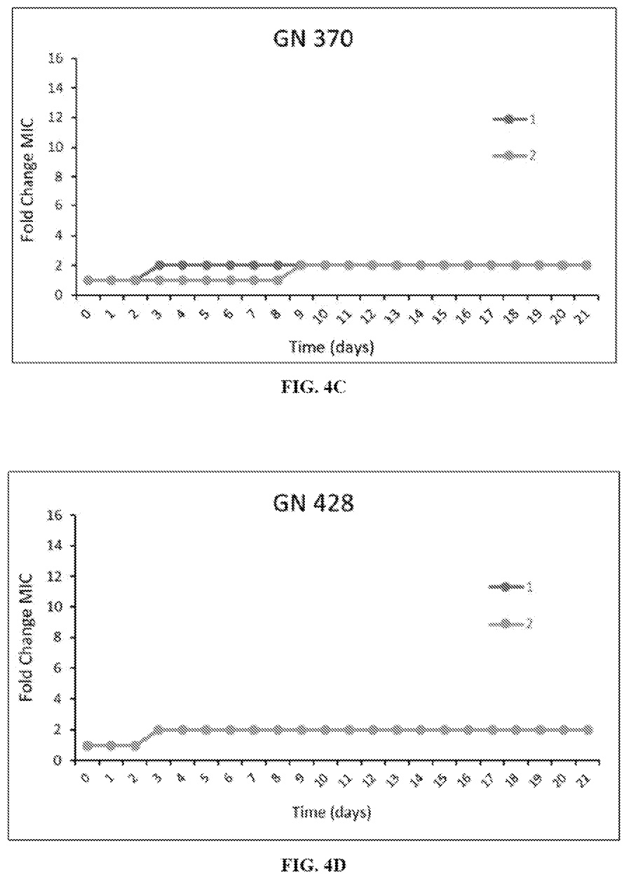

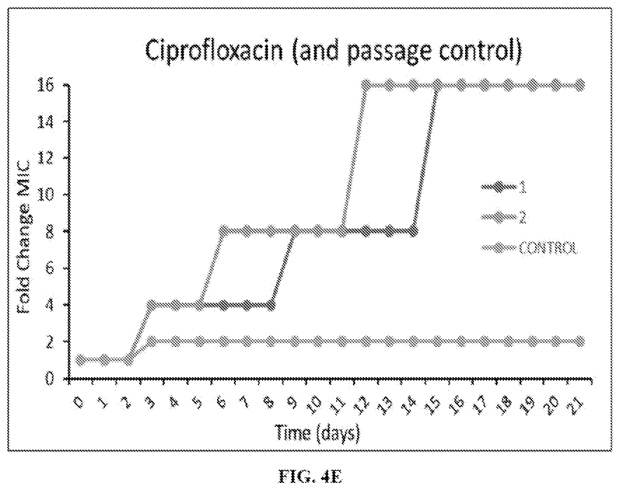

In the assay, GN121, GN351, GN370, and GN428 lysin MICs increased by up to 1-loge dilutions (2-fold) over 18 days, which was comparable to passage control (absence of treatment). FIGS. 4A-4D. In contrast, the Ciprofloxacin control increased 4-loge dilutions (16-fold) over 18 days (FIG. 4E). D'Lima et al. also found an increase in Ciprofloxacin MIC during serial passage. See D'Lima et al., 2012, Antimicrobial Agents and Chemotherapy, 56: 2753-2755, which reports an increase of Ciprofloxacin MIC of up to 32-fold over a 21 day serial passage. Our results are consistent with a low propensity for GN lysin resistance, which is similar to that observed with Gram-positive lysins. See, for example, PCT/US19/19638, which was filed on Feb. 26, 2019, and is herein incorporated by reference in its entirety.

1-36. (canceled)

37. A method for resensitizing a Gram-negative bacteria to an antibiotic suitable for the treatment of a Gram-negative bacterial infection, comprising:
co-administering the antibiotic in combination with a composition containing an effective amount of an isolated lysin and/or a lysin-antimicrobial peptide (AMP) polypeptide construct,
wherein the isolated lysin comprises at least one of:
(i) GN121 (SEQ ID NO: 175), GN123 (SEQ ID NO: 173), GN217 (SEQ ID NO: 8), GN316 variant (SEQ ID NO: 24), GN316 (SEQ ID NO: 22), GN329 (SEQ ID NO: 26), GN333 (SEQ ID NO: 28), GN394 (SEQ ID NO: 48), GN396 (SEQ ID NO: 50), GN408 (SEQ ID NO: 52), GN418 (SEQ ID NO: 54), GN424 (SEQ ID NO: 56), GN425 (SEQ ID NO:58), GN428 (SEQ ID NO: 60), GN431 (SEQ ID NO: 64), GN486 (SEQ ID NO: 66), GN485 (SEQ ID NO: 68), Lysin PaP2_gp17 (SEQ ID NO: 96), or
(ii) an active fragment thereof, or
(iii) a polypeptide having lytic activity and at least 80% sequence identity with the polypeptide sequence of at least one of SEQ ID NOS: 175, 173, 8, 24, 22, 26, 28, 48, 50, 52, 54, 56, 58, 60, 64, 66, 68, or 96;
wherein the lysin-AMP polypeptide construct comprises:
(a) a first component comprising the polypeptide sequence of:
(i) a lysin selected from the group consisting of GN76 (SEQ ID NO: 203), GN4 (SEQ ID NO: 74), GN146 (SEQ ID NO: 78), GN14 (SEQ ID NO: 124), GN37 (SEQ ID NO: 84) optionally with a single pI-increasing mutation, GN316 (SEQ ID NO: 22) optionally with a single point mutation, lysin Pap2_gp17 (SEQ ID NO: 96), GN329 (SEQ ID NO: 26), GN424 (SEQ ID NO: 56), GN202 (SEQ ID NO: 118), GN425 (SEQ ID NO: 58), GN428 (SEQ ID NO: 60), GN431 (SEQ ID NO: 64), GN486 (SEQ ID NO: 66), GN333 (SEQ ID NO: 28), GN485 (SEQ ID NO: 68), GN123 (SEQ ID NO: 173) and GN121 (SEQ ID NO: 175); or
(ii) a polypeptide having lytic activity and having at least 80% sequence identity with the polypeptide sequence of at least one of SEQ ID NOS: 203, 74, 78, 124, 84, 22, 96, 26, 56, 118, 58, 60, 64, 66, 28, 68, 173 or 175; or
(iii) an active fragment of the lysin; and
(b) a second component comprising the polypeptide sequence of:
(i) at least one antimicrobial peptide (AMP) selected from the group consisting of Chp1 (SEQ ID NO: 133), Chp2 (SEQ ID NO: 70), CPAR39 (SEQ ID NO: 135), Chp3 (SEQ ID NO: 137), Chp4 (SEQ ID NO: 102), Chp6 (SEQ ID NO: 106), Chp7 (SEQ ID NO: 139), Chp8 (SEQ ID NO: 141), Chp9 (SEQ ID NO: 143), Chp10 (SEQ ID NO: 145), Chp11 (SEQ ID NO: 147), Chp12 (SEQ ID NO: 149), Gkh1 (SEQ ID NO: 151), Gkh2 (SEQ ID NO: 90), Unp1 (SEQ ID NO: 153), Ecp1 (SEQ ID NO: 155), Ecp2 (SEQ ID NO: 104), Tma1 (SEQ ID NO: 157), Osp1 (SEQ ID NO: 108), Unp2 (SEQ ID NO: 159), Unp3 (SEQ ID NO: 161), Gkh3 (SEQ ID NO: 163), Unp5 (SEQ ID NO: 165), Unp6 (SEQ ID NO: 167), Spi1 (SEQ ID NO: 169), Spi2 (SEQ ID NO: 171), Ecp3 (SEQ ID NO: 177), Ecp4 (SEQ ID NO: 179), ALCES1 (SEQ ID NO: 181), AVQ206 (SEQ ID NO: 183), AVQ244 (SEQ ID NO: 185), CDL907 (SEQ ID NO: 187), AGT915 (SEQ ID NO: 189), HH3930 (SEQ ID NO: 191), Fen7875 (SEQ ID NO: 193), SBR77 (SEQ ID NO: 195), Bdp1 (SEQ ID NO: 197), LVP1 (SEQ ID NO: 199), Lvp2 (SEQ ID NO: 201), an esculentin fragment (SEQ ID NO: 80), RI12 (SEQ ID NO: 88), TI15 (SEQ ID NO: 94), RI18 (SEQ ID NO: 92), FIRL (SEQ ID NO: 114), a fragment of LPS binding protein (SEQ ID NO: 76), RR12whydro (SEQ ID NO: 110), RI18 peptide derivative (SEQ ID NO: 131) and cationic peptide (SEQ ID NO: 120) or
(ii) a polypeptide having AMP activity, wherein the polypeptide is at least 80% identical to at least one of SEQ ID NOS: 133, 70, 135, 137, 102, 106, 139, 141, 143, 145, 147, 149, 151, 90, 153, 155, 104, 157, 108, 159, 161, 163, 165, 167, 169, 171, 177, 179, 181, 183, 185, 187, 189, 191, 193, 195, 197, 199, 201, 80, 88, 94, 92, 114, 76, 110, 131 and 120,
wherein the composition comprises at least one activity selected from inhibiting P. aeruginosa bacterial growth, reducing a P. aeruginosa bacterial population and/or killing P. aeruginosa in the presence of pulmonary surfactant, and
wherein administration of the combination resensitizes the Gram-negative bacteria to the antibiotic.

38. (canceled)

39. The method of claim 37, wherein the Gram-negative bacteria is selected from the group consisting of Pseudomonas aeruginosa, Klebsiella spp., Enterobacter spp., Escherichia coli, Citrobacter freundii, Salmonella typhimurium, Yersinia pestis, and Franciscella tulerensis.

40. The method of claim 37, wherein the antibiotic is selected from one or more of ceftazidime, cefepime, cefoperazone, ceftobiprole, ciprofloxacin, levofloxacin, aminoglycosides, imipenem, meropenem, doripenem, gentamicin, tobramycin, amikacin, piperacillin, ticarcillin, penicillin, rifampicin, polymyxin B, and colistin.

41. The method of claim 37, wherein the at least one activity further comprises inhibiting the growth, or reducing a population of at least one species of Gram-negative bacteria in addition to P. aeruginosa.

42. (canceled)

43. The method of claim 37, wherein the lysin-AMP polypeptide construct or the isolated polypeptide resensitizes P. aeruginosa to an antibiotic.

44. The method of claim 37, wherein the antibiotic is a carbapenem.

45. The method of claim 37, wherein the antibiotic is meropenem.

46.-57. (canceled)

58. The method of claim 37, wherein the lysin-AMP polypeptide construct comprises a first component that is selected from the group consisting of GN394 (SEQ ID NO: 48), GN396 (SEQ ID NO: 50), GN408 (SEQ ID NO: 52), GN418 (SEQ ID NO: 54), and GN202 (SEQ ID NO: 118).

59. The method of claim 37, wherein the lysin-AMP polypeptide construct further comprises at least one structure stabilizing component to maintain at least a portion of the structure of the first and/or second component in the construct substantially the same as in an unconjugated lysin and/or AMP.

60. The method of claim 59, wherein the at least one structure stabilizing component is a peptide.

61. The method of claim 60, wherein the peptide is selected from the group consisting of TAGGTAGG (SEQ ID NO: 72), IGEM (BBa_K1485002) (SEQ ID NO: 82), PPTAGGTAGG (SEQ ID NO: 98), IGEM +PP (residues 44-58 of SEQ ID NO: 16) and AGAGAGAGAGAGAGAGAS (SEQ ID NO: 122).

62. The method of claim 37, wherein the lysin-AMP polypeptide construct comprises (i) a polypeptide sequence selected from the group consisting of GN168 (SEQ ID NO: 2), GN176 (SEQ ID NO: 4), GN178 (SEQ ID NO: 6) GN218 (SEQ ID NO: 10), GN223 (SEQ ID NO: 12), GN239 (SEQ ID NO: 14), GN243 (SEQ ID NO: 16), GN280 (SEQ ID NO: 18), GN281 (SEQ ID NO: 20), GN349 (SEQ ID NO: 30), GN351 (SEQ ID NO: 32), GN352 (SEQ ID NO: 34), GN353 (SEQ ID NO: 36), GN357 (SEQ ID NO: 38), GN359 (SEQ ID NO: 40), GN369 (SEQ ID NO: 42), GN370 (SEQ ID NO: 44), GN371 (SEQ ID NO: 46), GN428 (SEQ ID NO: 60), and GN93 (SEQ ID NO: 62), or (ii) a polypeptide having lysin activity and at least 80% identity with at least one of SEQ ID NOS: 2, 4,6, 10, 12, 14, 16, 18, 20, 30, 32, 34, 36, 38, 40, 42, 44, 46, 60 and 62.

63. The method of claim 37, wherein the lysin-AMP polypeptide construct comprises (i) a polypeptide sequence selected from the group consisting of GN351 (SEQ ID NO: 32) and GN370 (SEQ ID NO: 36), or (ii) a polypeptide having lysin activity and at least 80% identity with at least one of SEQ ID NOS: 32 and 36.

64. The method of claim 37, wherein the isolated lysin is selected from the group consisting of GN121 (SEQ ID NO: 175), GN217 lysin (SEQ ID NO: 8), GN394 lysin (SEQ ID NO: 48), GN396 lysin (SEQ ID NO: 50), GN408 lysin (SEQ ID NO: 52), GN418 lysin (SEQ ID NO: 54), GN428 (SEQ ID NO: 60), and GN486 (SEQ ID NO: 66) or an active fragment thereof, wherein the lysin or active fragment thereof comprises at least one activity selected from inhibiting P. aeruginosa bacterial growth, reducing a P. aeruginosa bacterial population and/or killing P. aeruginosa in the presence of pulmonary surfactant.

65. The method of claim 64, wherein the isolated lysin is selected from the group consisting of GN121 (SEQ ID NO: 175) and GN428 (SEQ ID NO: 60).
